SEQUENCE LISTING (90 sequences)

SEQ ID NO: 1 - DNA, 21 bp, Mus musculus
  source 1..21: mol_type = other DNA
ttttgggatggaattggacac

SEQ ID NO: 2 - DNA, 20 bp, Mus musculus
  source 1..20: mol_type = other DNA
tgcaggtgaagccttcaacc

SEQ ID NO: 3 - DNA, 20 bp, Mus musculus
  source 1..20: mol_type = other DNA
tgaaaaccccaactccaaag

SEQ ID NO: 4 - DNA, 20 bp, Mus musculus
  source 1..20: mol_type = other DNA
cattccttaggcgtgaccat

SEQ ID NO: 5 - DNA, 18 bp, Mus musculus
  source 1..18: mol_type = other DNA
cgcagtgcttctttgcct

SEQ ID NO: 6 - DNA, 20 bp, Mus musculus
  source 1..20: mol_type = other DNA
tctggcccagtcagcttctc

SEQ ID NO: 7 - DNA, 23 bp, Mus musculus
  source 1..23: mol_type = other DNA
gcacatagagagaatgagcttcc

SEQ ID NO: 8 - DNA, 19 bp, Mus musculus
  source 1..19: mol_type = other DNA
ctccgctctgaacaaggct

SEQ ID NO: 9 - DNA, 22 bp, Mus musculus
  source 1..22: mol_type = other DNA
tccagagcttgaaggtgttgcc

SEQ ID NO: 10 - DNA, 21 bp, Mus musculus
  source 1..21: mol_type = other DNA
aaccaagggagcttcagggtc

SEQ ID NO: 11 - DNA, 20 bp, Mus musculus
  source 1..20: mol_type = other DNA
ggctgtattcccctccatcg

SEQ ID NO: 12 - DNA, 22 bp, Mus musculus
  source 1..22: mol_type = other DNA
ccagttggtaacaatgccatgt

SEQ ID NO: 13 - DNA, 20 bp, Mus musculus
  source 1..20: mol_type = other DNA
tgatgcacttgcagaaaaca

SEQ ID NO: 14 - DNA, 23 bp, Mus musculus
  source 1..23: mol_type = other DNA
accagaggaaattttcaataggc

SEQ ID NO: 15 - DNA, 20 bp, Mus musculus
  source 1..20: mol_type = other DNA
tgccctgaactgctttctcc

SEQ ID NO: 16 - DNA, 20 bp, Mus musculus
  source 1..20: mol_type = other DNA
tctcctccagctcctcacat

SEQ ID NO: 17 - DNA, 20 bp, Mus musculus
  source 1..20: mol_type = other DNA
gaggaaatggatccacctga

SEQ ID NO: 18 - DNA, 20 bp, Mus musculus
  source 1..20: mol_type = other DNA
atcttccttttggcaagcaa

SEQ ID NO: 19 - DNA, 19 bp, Mus musculus
  source 1..19: mol_type = other DNA
cctgctgttcacagttgcc

SEQ ID NO: 20 - DNA, 20 bp, Mus musculus
  source 1..20: mol_type = other DNA
attgggatcatcttgctggt

SEQ ID NO: 21 - DNA, 20 bp, Mus musculus
  source 1..20: mol_type = other DNA
gccctctctctcctcttgct

SEQ ID NO: 22 - DNA, 20 bp, Mus musculus
  source 1..20: mol_type = other DNA
gtctgcctcttttggtcagg

SEQ ID NO: 23 - DNA, 20 bp, Mus musculus
  source 1..20: mol_type = other DNA
gtgagaatgagggccatagg

SEQ ID NO: 24 - DNA, 21 bp, Mus musculus
  source 1..21: mol_type = other DNA
tttttggctaaacgctttcat

SEQ ID NO: 25 - DNA, 20 bp, Mus musculus
  source 1..20: mol_type = other DNA
agtgaactgcgctgtcaatg

SEQ ID NO: 26 - DNA, 20 bp, Mus musculus
  source 1..20: mol_type = other DNA
ttcagggtcaaggcaaactt

SEQ ID NO: 27 - DNA, 19 bp, Mus musculus
  source 1..19: mol_type = other DNA
ctgctacctgcactttgcc

SEQ ID NO: 28 - DNA, 20 bp, Mus musculus
  source 1..20: mol_type = other DNA
tggatacctgagcatcacca

SEQ ID NO: 29 - DNA, 21 bp, Mus musculus
  source 1..21: mol_type = other DNA
ggaaagatgatggggaatttg

SEQ ID NO: 30 - DNA, 20 bp, Mus musculus
  source 1..20: mol_type = other DNA
gaagcggggaagttgatgta

SEQ ID NO: 31 - DNA, 20 bp, Mus musculus
  source 1..20: mol_type = other DNA
ggaaagatgatggggacctt

SEQ ID NO: 32 - DNA, 20 bp, Mus musculus
  source 1..20: mol_type = other DNA
gaagcggggaagttgatgta

SEQ ID NO: 33 - DNA, 22 bp, Mus musculus
  source 1..22: mol_type = other DNA
agttgggttctgctccgtggag

SEQ ID NO: 34 - DNA, 21 bp, Mus musculus
  source 1..21: mol_type = other DNA
atcccaacgccctgaaccgct

SEQ ID NO: 35 - DNA, 23 bp, Mus musculus
  source 1..23: mol_type = other DNA
tgttgaggctagagaggatcttg

SEQ ID NO: 36 - DNA, 22 bp, Mus musculus
  source 1..22: mol_type = other DNA
cgaatctgcaccgtagttgagc

SEQ ID NO: 37 - DNA, 20 bp, Mus musculus
  source 1..20: mol_type = other DNA
gtgcccacgtcaaggagtat

SEQ ID NO: 38 - DNA, 20 bp, Mus musculus
  source 1..20: mol_type = other DNA
ccacttcttctctgggttgg

SEQ ID NO: 39 - DNA, 21 bp, Mus musculus
  source 1..21: mol_type = other DNA
tctctgtcctcggaagttgaa

SEQ ID NO: 40 - DNA, 21 bp, Mus musculus
  source 1..21: mol_type = other DNA
gggatcccatcaatatcgtct

SEQ ID NO: 41 - DNA, 19 bp, Mus musculus
  source 1..19: mol_type = other DNA
gagccaccagcaagagcaa

SEQ ID NO: 42 - DNA, 22 bp, Mus musculus
  source 1..22: mol_type = other DNA
ccagtatggtccccagatagct

SEQ ID NO: 43 - DNA, 20 bp, Mus musculus
  source 1..20: mol_type = other DNA
gggtctgagtttcacctgga

SEQ ID NO: 44 - DNA, 20 bp, Mus musculus
  source 1..20: mol_type = other DNA
cccagcacagcttgttatga

SEQ ID NO: 45 - DNA, 20 bp, Mus musculus
  source 1..20: mol_type = other DNA
gcagtggcaaagtggagatt

SEQ ID NO: 46 - DNA, 20 bp, Mus musculus
  source 1..20: mol_type = other DNA
gaatttgccgtgagtggagt

SEQ ID NO: 47 - DNA, 23 bp, Homo sapiens
  source 1..23: mol_type = other DNA
aacagaagccaactgggtgaatg

SEQ ID NO: 48 - DNA, 24 bp, Homo sapiens
  source 1..24: mol_type = other DNA
ctccaagagaaagcacttcattgc

SEQ ID NO: 49 - DNA, 22 bp, Homo sapiens
  source 1..22: mol_type = other DNA
agacagccactcacctcttcag

SEQ ID NO: 50 - DNA, 21 bp, Homo sapiens
  source 1..21: mol_type = other DNA
ttctgccagtgcctctttgct

SEQ ID NO: 51 - DNA, 22 bp, Homo sapiens
  source 1..22: mol_type = other DNA
gatagccagcctagaggtatgg

SEQ ID NO: 52 - DNA, 24 bp, Homo sapiens
  source 1..24: mol_type = other DNA
ccttgatgttatcaggaggattca

SEQ ID NO: 53 - DNA, 22 bp, Homo sapiens
  source 1..22: mol_type = other DNA
ggcagaaagcttgtctcaaccc

SEQ ID NO: 54 - DNA, 21 bp, Homo sapiens
  source 1..21: mol_type = other DNA
ctccttcaggaacagccacca

SEQ ID NO: 55 - DNA, 21 bp, Homo sapiens
  source 1..21: mol_type = other DNA
gcccaaaccgaagtcatagcc

SEQ ID NO: 56 - DNA, 20 bp, Homo sapiens
  source 1..20: mol_type = other DNA
atccgccagcctctatcaca

SEQ ID NO: 57 - DNA, 23 bp, Homo sapiens
  source 1..23: mol_type = other DNA
ggtgagaagagatgtctgaatcc

SEQ ID NO: 58 - DNA, 22 bp, Homo sapiens
  source 1..22: mol_type = other DNA
gtccatccttggaagcactgca

SEQ ID NO: 59 - DNA, 22 bp, Homo sapiens
  source 1..22: mol_type = other DNA
tatccagaggctggagagctac

SEQ ID NO: 60 - DNA, 22 bp, Homo sapiens
  source 1..22: mol_type = other DNA
tggaatccctgacccatctctc

SEQ ID NO: 61 - DNA, 23 bp, Homo sapiens
  source 1..23: mol_type = other DNA
aagttgtctgtgtgcgcaaatcc

SEQ ID NO: 62 - DNA, 24 bp, Homo sapiens
  source 1..24: mol_type = other DNA
ccattccagaaaagccacagtttt

SEQ ID NO: 63 - DNA, 23 bp, Homo sapiens
  source 1..23: mol_type = other DNA
gcttcctcgcaactttgtggtag

SEQ ID NO: 64 - DNA, 22 bp, Homo sapiens
  source 1..22: mol_type = other DNA
ggtcatacacgtactcctggac

SEQ ID NO: 65 - DNA, 22 bp, Homo sapiens
  source 1..22: mol_type = other DNA
acagaaggaccaccagtagcca

SEQ ID NO: 66 - DNA, 23 bp, Homo sapiens
  source 1..23: mol_type = other DNA
ggtgcttcataaagtcctggacc

SEQ ID NO: 67 - DNA, 20 bp, Homo sapiens
  source 1..20: mol_type = other DNA
ttctgtgcctgctgctcata

SEQ ID NO: 68 - DNA, 20 bp, Homo sapiens
  source 1..20: mol_type = other DNA
agcttctttgggacacttgc

SEQ ID NO: 69 - DNA, 20 bp, Homo sapiens
  source 1..20: mol_type = other DNA
agcttctcctgctctgcaac

SEQ ID NO: 70 - DNA, 19 bp, Homo sapiens
  source 1..19: mol_type = other DNA
acacttgagctcgggcaat

SEQ ID NO: 71 - DNA, 20 bp, Homo sapiens
  source 1..20: mol_type = other DNA
gggagaagaacagggatgtg

SEQ ID NO: 72 - DNA, 20 bp, Homo sapiens
  source 1..20: mol_type = other DNA
atgagaaggctgcagggact

SEQ ID NO: 73 - DNA, 20 bp, Homo sapiens
  source 1..20: mol_type = other DNA
tggaaagcacagaacccagt

SEQ ID NO: 74 - DNA, 20 bp, Homo sapiens
  source 1..20: mol_type = other DNA
ctgcccatcgaaactgaact

SEQ ID NO: 75 - DNA, 20 bp, Homo sapiens
  source 1..20: mol_type = other DNA
tagcggactgaccaccttct

SEQ ID NO: 76 - DNA, 20 bp, Homo sapiens
  source 1..20: mol_type = other DNA
ggatgatgaggaagctccag

SEQ ID NO: 77 - DNA, 20 bp, Homo sapiens
  source 1..20: mol_type = other DNA
ggctcagggaattcttaggg

SEQ ID NO: 78 - DNA, 20 bp, Homo sapiens
  source 1..20: mol_type = other DNA
cattttgccaccagacacag

SEQ ID NO: 79 - DNA, 20 bp, Homo sapiens
  source 1..20: mol_type = other DNA
cctcccctgcttcatcctac

SEQ ID NO: 80 - DNA, 20 bp, Homo sapiens
  source 1..20: mol_type = other DNA
gctgcgccgttatttattgt

SEQ ID NO: 81 - DNA, 20 bp, Homo sapiens
  source 1..20: mol_type = other DNA
gcgatgaacgtcaggaattt

SEQ ID NO: 82 - DNA, 20 bp, Homo sapiens
  source 1..20: mol_type = other DNA
ctgcatgtcacggtgatgtt

SEQ ID NO: 83 - DNA, 20 bp, Homo sapiens
  source 1..20: mol_type = other DNA
ctggtgcttcagagtcaacg

SEQ ID NO: 84 - DNA, 20 bp, Homo sapiens
  source 1..20: mol_type = other DNA
ctgtggtctcctgtcccaac

SEQ ID NO: 85 - DNA, 20 bp, Homo sapiens
  source 1..20: mol_type = other DNA
ggaaagctagggactgcaca

SEQ ID NO: 86 - DNA, 20 bp, Homo sapiens
  source 1..20: mol_type = other DNA
cgggtgcagtttatttccac

SEQ ID NO: 87 - DNA, 20 bp, Homo sapiens
  source 1..20: mol_type = other DNA
ctgctgctttgcctacctct

SEQ ID NO: 88 - DNA, 20 bp, Homo sapiens
  source 1..20: mol_type = other DNA
cgagtgacaaacacgactgc

SEQ ID NO: 89 - DNA, 20 bp, Homo sapiens
  source 1..20: mol_type = other DNA
gtcagtggtggacctgacct

SEQ ID NO: 90 - DNA, 20 bp, Homo sapiens
  source 1..20: mol_type = other DNA
tcgctgttgaactcagagga

(19) United States
(12) Patent Application Publication
(10) Pub. No.: US 2024/0390379 A1
(43) Pub. Date: Nov. 28, 2024

(54) EZH2 Inhibition in Pancreatic Cancer

(71) Applicant: University of Massachusetts, Boston, MA (US)

(72) Inventors: Marcus RUSCETTI, Marlborough, MA (US); Loretah CHIBAYA, Worchester, MA (US)

(21) Appl. No.: 18/694,552

(22) PCT Filed: Sep. 29, 2022
(86) PCT No.: PCT/US2022/045163

§ 371 Date: Mar. 22, 2024

Related U.S. Application Data
(60) Provisional application No. 63/249,716, filed on Sep. 29, 2021.

(51) Int. Cl. A61K 31/519; A61K 31/166; A61K 31/18; A61K 31/277; A61K 31/282; A61K 31/337; A61K 31/404; A61K 31/4184; A61K 31/436; A61K 31/44; A61K 31/4523; A61K 31/4745; A61K 31/496; A61K 31/506; A61K 31/513; A61K 31/517; A61K 31/5377; A61K 31/7068; A61K 33/243; A61K 39/395; A61P 35/00

(52) U.S. Cl. CPC A61K 31/519; A61K 31/166; A61K 31/18; A61K 31/277; A61K 31/282; A61K 31/337; A61K 31/404; A61K 31/4184; A61K 31/436; A61K 31/44; A61K 31/4523; A61K 31/4745; A61K 31/496; A61K 31/506; A61K 31/513; A61K 31/517; A61K 31/5377; A61K 31/7068; A61K 33/243; A61K 39/3955; A61P 35/00

(57) ABSTRACT

Described herein are methods for preventing or treating KRAS mutant pancreatic cancer in a subject in need thereof comprising administering to the subject an effective amount of (i) an effective amount of a PRC2 inhibitor, (ii) an effective amount of a MEK inhibitor, (ii) and an effective amount of a CDK4/6 inhibitor, as well as compositions and kits for use in said methods.

### CLAIM OF PRIORITY

This application claims the benefit of U.S. Patent Application Ser. No. 63/249,716, filed on Sep. 29, 2021. The entire contents of the foregoing are hereby incorporated by reference.

### FEDERALLY SPONSORED RESEARCH OR DEVELOPMENT

This invention was made with Government support under Grant No. CA241110 awarded by the National Institutes of Health. The Government has certain rights in the invention.

### TECHNICAL FIELD

Described herein are methods for preventing or treating KRAS mutant pancreatic cancer in a subject in need thereof comprising administering to the subject an effective amount of (i) an effective amount of a PRC2 inhibitor, (ii) an effective amount of a MEK inhibitor, (ii) and an effective amount of a CDK4/6 inhibitor, as well as compositions and kits for use in said methods.

### BACKGROUND

Pancreatic cancer was the 12^{th} most common type of cancer in the U.S. in 2014, representing about 2.8% of all new cancer cases. However, pancreatic cancer was the 4^{th} most common cause of cancer-related deaths (Schneider G et al., Gastroenterology 128(6):1606-1625 (2005)). In 2014, about 46,420 new cases and 39,590 deaths were attributable to pancreatic cancer in the United States, of which pancreatic ductal adenocarcinoma (PDAC) represents the vast majority. The fact that the annual number of pancreatic cancer-related deaths nearly equals the annual number of new pancreatic cancer cases highlights the lethality of this disease. PDAC, the most common malignancy of the pancreas, is both aggressive and difficult to treat. Complete surgical removal of the tumor remains the only chance for cure, however 80-90% of patients have disease that is surgically incurable at the time of clinical presentation (Schneider G et al., Gastroenterology 128(6):1606-1625 (2005)). Accordingly, there is an urgent need for effective therapies for pancreatic cancer.

### SUMMARY

PDAC remains without durable chemo-, targeted, and immunotherapy regimens, and as such has a dismal 5-year survival rate of 11%¹. Many promising studies and clinical trials have focused on overcoming immune suppression as a therapeutic strategy in PDAC through (a) re-engineering T cell responses via CAR-T, ICB therapy, or neo-antigen vaccine approaches, (b) targeting suppressive fibroblast populations and functions, and/or (c) eliminating or reprogramming suppressive myeloid cells⁵⁹. Here we investigated how to remodel the tumor secretome directly as a strategy to enhance tumor immunogenicity and transform the immune suppressive PDAC TME. By comparing the effects of therapy-induced senescence on KRAS mutant LUAD and PDAC tumors, we uncovered an epigenetic mechanism that suppresses the pro-inflammatory SASP secretome in PDAC that is mediated by PRC2 component EZH2 and its methyltransferase activity. EZH2 inhibition in combination with therapy-induced senescence unleashes pro-inflammatory SASP chemokines such as CCL2 and CXCL9/10 and induces MHC-I and NK ligand expression to orchestrate an innate and adaptive immune attack through cytotoxic NK and T lymphocytes that in some cases led to complete responses in preclinical PDAC models.

Described herein are methods for preventing or treating KRAS mutant pancreatic cancer in a subject in need thereof comprising administering to the subject an effective amount of (i) an effective amount of a PRC2 inhibitor, (ii) an effective amount of a MEK inhibitor, (ii) and an effective amount of a CDK4/6 inhibitor. Also provided herein are (i) an effective amount of a PRC2 inhibitor, (ii) an effective amount of a MEK inhibitor, (ii) and an effective amount of a CDK4/6 inhibitor, in a single or more than one composition, for use in methods for preventing or treating KRAS mutant pancreatic cancer in a subject.

In some embodiments, the MEK inhibitor is an inhibitory nucleic acid or a small molecule inhibitor, preferably selected from the group consisting of trametinib, cobimetinib, binimetinib, selumetinib, PD-325901, TAK-733, Cl-1040 (PD 184352), PD0325901, MEK162, AZD8330, GDC-0623, refametinib, pimasertib, R04987655, R05126766, WX-554, HL-085, CInQ-03, G-573, PD184161, PD318088, PD98059, R05068760, U0126, and SL327.

In some embodiments, the CDK4/6 inhibitor is an inhibitory nucleic acid or a small molecule inhibitor, preferably selected from the group consisting of palbociclib, ribociclib, and abemaciclib.

In some embodiments, the PRC2 inhibitor is an inhibitory nucleic acid or a small molecule inhibitor, optionally an inhibitor of EZH2 or EED as described herein, preferably GSK126 or Tazemetostat.

In some embodiments, the methods comprise administering Trametinib; palbociclib; and GSK126 or Tazemetostat.

In some embodiments, the subject is non-responsive to at least one prior line of cancer therapy. In some embodiments, the at least one prior line of cancer therapy is chemotherapy or immunotherapy.

In some embodiments, the pancreatic cancer is an exocrine pancreatic cancer or an endocrine pancreatic cancer. In some embodiments, the pancreatic cancer is selected from the group consisting of pancreatic ductal adenocarcinoma (PDAC), acinar cell carcinoma, solid pseudopapillary neoplasms, pancreatoblastoma, pancreatic neuroendocrine tumors (PNETs), gastrinomas, insulinomas, glucagonomas, somatostatinomas and VIPomas.

In some embodiments, the KRAS mutation is G12D, G12V, G12C, G12R, G12A, G13D, Q61L or Q61H.

In some embodiments, the PRC2 inhibitor, MEK inhibitor, and CDK4/6 inhibitor are administered sequentially, simultaneously, or separately.

In some embodiments, the PRC2 inhibitor, MEK inhibitor, and/or CDK4/6 inhibitor is administered orally, intraperitoneally, or intravenously.

In some embodiments, the subject is a mammal, e.g., a human or non-human veterinary subject.

In some embodiments, the subject exhibits an increase in one or more of (a) NK cell immune surveillance, (b) senescent tumor cell clearance, or (c) vascular re-normalization after administration of the PRC2 inhibitor, MEK inhibitor, and CDK4/6 inhibitor.

In some embodiments, the subject exhibits a delay in metastatic onset and/or tumor growth after administration of the PRC2 inhibitor, MEK inhibitor, and CDK4/6 inhibitor compared to that observed in an untreated control subject diagnosed with pancreatic cancer.

In some embodiments, the methods further include administering at least one chemotherapeutic agent in a patient with pancreatic cancer comprising. In some embodiments, the at least one chemotherapeutic agent is selected from the group consisting of abraxane, capecitabine, erlotinib, fluorouracil (5-FU), gemcitabine, irinotecan, leucovorin, nab-paclitaxel, cisplatin, irinotecan, docetaxel, oxaliplatin, tipifamib, everolimus, sunitinib, dovitinib, ruxolitinib, pegylated-hyaluronidase, pemetrexed, folinic acid, paclitaxel, MK2206, GDC-0449, IPI-926, gamma secretase/R04929097, M402, and LY293111.

In some embodiments, the methods further include administering at least one immunotherapeutic agent. In some embodiments, the at least one immunotherapeutic agent is selected from the group consisting of immune checkpoint inhibitors (e.g., anti-CD137 (BMS-663513), anti-PD1 (e.g., Nivolumab, pembrolizumab/MK-3475, Pidilizumab (CT-011)), anti-PDL1 (e.g., BMS-936559, MPDL3280A), or anti-CTLA-4 (e.g., ipilumimab)), sipuleucel-T, CRS-207, and GVAX, or is a monoclonal antibody selected from ipilimumab, 90Y-Clivatuzumab tetraxetan, pembrolizumab, nivolumab, trastuzumab, cixutumumab, ganitumab, demcizumab, cetuximab, nimotuzumab, and dalotuzumab.

Also provided herein are kits comprising a PRC2 inhibitor, a MEK inhibitor, a CDK4/CDK6 inhibitor, and optionally instructions for use in a method described herein, e.g., for treating pancreatic cancer.

Additionally, provided herein are pharmaceutical compositions comprising a PRC2 inhibitor and one or both of a MEK inhibitor and/or a CDK4/6 inhibitor, and a pharmaceutically acceptable excipient or carrier.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-E. NK cell immunity is induced in the lung but not pancreas TME following therapy-induced senescence. a-b, KPC1 PDAC (a) or KP1 LUAD (b) tumor cells expressing luciferase-GFP were injected i.v. or orthotopically into the pancreas of C57BL/6 mice. Following tumor formation in the lungs or pancreas, mice were treated with vehicle (V) or combined trametinib (1 mg/kg body weight) and palbociclib (100 mg/kg body weight) (T/P) for 2 weeks (left). Right, flow cytometry analysis of NK cell numbers and degranulation in each condition (n≥3 per group). c, KPC1 PDAC or KP1 LUAD cells expressing luciferase-GFP were injected orthotopically into the livers of C57BL/6 mice and treated as in (a) following tumor formation (left). Right, flow cytometry analysis of NK cell numbers and degranulation in each condition following 2-week treatment (n≥8 per group). d, Kaplan-Meier survival curve of C57BL/6 mice harboring KPC1 PDAC tumors in lungs (PIL) treated with vehicle or trametinib (1 mg/kg) and palbociclib (100 mg/kg) in the presence or absence of a NK1.1 depleting antibody (PK136; 250 ug) (n≥8 per group). e, Kaplan-Meier survival curve of C57BL/6 mice harboring KP1 LUAD tumors in pancreas (LIP) and treated as in (d) (n≥7 per group). PIP, PDAC tumors in pancreas; PIL, PDAC tumors in lungs; LIL, LUAD tumors in lungs; LIP, LUAD tumors in pancreas. PILiver, PDAC tumors in liver; LILiver, LUAD tumors in liver. P values in a-c were calculated using two-tailed, unpaired Student's t-test, and those in d and e calculated using log-rank test. Error bars, mean±SEM. **** P<0.0001, *** P<0.001, * P<0.05. n.s., not significant.

FIGS. 2A-F. The pancreas TME suppresses the pro-inflammatory SASP. a, KPC1 PDAC or KP1 LUAD tumor cells expressing luciferase-GFP were injected i.v. or orthotopically into the pancreas of C57BL/6 mice. Following tumor formation in the lungs or pancreas, mice were treated with vehicle (V) or combined trametinib (1 mg/kg) and palbociclib (100 mg/kg) (T/P) for 2 weeks (left). GFP⁺ tumor cells were FACS sorted and extracted RNA subjected to RNA-seq analysis (n=2-4 per group). b, KEGG pathway analysis of pathways enriched in tumors in the lungs (LIL, PIL) compared to tumors in the pancreas (PIP, LIP) following T/P treatment. c, Heatmap showing fold change in SASP gene expression following T/P treatment in indicated tumor settings. d, Fold change in expression of select SASP chemokines following T/P treatment in indicated tumor settings. e-f, Transcription factor enrichment analysis showing transcriptional regulators whose targets are enriched in tumors in the lungs (LIL, PIL) (e) or in the pancreas (PIP, LIP) (f) following T/P treatment.

FIGS. 3A-E. EZH2 methyltransferase activity leads to pro-inflammatory SASP suppression in PDAC. a, Immunoblots of KPC1 and KPC2 PDAC cells harboring Renilla (Ren), Ezh2, or Suz12 shRNAs. b, Cytokine array results from KPC1 and KPC2 PDAC cells with indicated shRNAs treated for 8 days with vehicle or trametinib (25 nM) and palbociclib (500 nM) (n=3). c, qRT-PCR analysis of senescence and SASP gene expression in KPC1 PDAC cells treated with vehicle, trametinib (25 nM), palbociclib (500 nM), GSK126 (1 μM), and/or tazemetostat (5 μM) for 8 days (n=3-6 per group). A.U., arbitrary units. d, Schematic of the CUT & Tag protocol used in (e). e, Genome browser tracks from CUT & Tag analysis showing H3K27me3 occupancy at select pro-inflammatory SASP gene loci in KPC1 PDAC cells harboring Ren or Ezh2 shRNAs treated with vehicle or trametinib (25 nM) and palbociclib (500 nM) for 8 days (n=2-4 per group). P values in b-c were calculated using two-tailed, unpaired Student's t-test. Error bars, mean±SEM. **** P<0.0001, *** P<0.001, ** P<0.01, * P<0.05. n.s., not significant.

FIGS. 4A-I. EZH2 blockade activates NK and T cell-mediated long-term tumor control following therapy-induced senescence in PDAC models. a, Schematic of KPC PDAC syngeneic orthotopic transplant model and treatment regimens. b, Representative flow cytometry plots of CD45⁺CD3⁻NK1.1⁺ NK cells in KPC1 orthotopic PDAC tumors harboring indicated shRNAs from mice treated with vehicle (V) or combined trametinib (1 mg/kg) and palbociclib (100 mg/kg) (T/P) for 2 weeks. SSC, side scatter. c-e, Flow cytometry analysis of total CD45⁺ immune cells (c), NK cell numbers and activation markers (d), and T cell numbers and activation markers (e) in KPC1 orthotopic PDAC tumors harboring indicated shRNAs following treatment as in (b) (n≥4 per group). f, Waterfall plot of the response of KPC1 orthotopic PDAC tumors with indicated shRNAs to treatment as in (b) (n≥10 per group). g, Representative ultrasound images of shEzh2 KPC1 orthotopic PDAC tumors prior to treatment and after 2 or 15 weeks of treatment with combined trametinib (1 mg/kg) and palbociclib (100 mg/kg). PDAC tumors are outlined in white. h, Kaplan-Meier survival curve of mice with KPC1 orthotopic PDAC tumors harboring indicated shRNAs treated with vehicle, combined trametinib (1 mg/kg) and palbociclib (100 mg/kg), and/or an NK1.1 depleting antibody (PK136; 250 μg) (n≥5 per group). i, Kaplan-Meier survival curve of mice with shEzh2 KPC1 orthotopic PDAC tumors treated with vehicle, combined trametinib (1 mg/kg) and palbociclib (100 mg/kg), and/or a CD8 depleting antibody (2.43; 200 μg) (n≥6 per group). Dotted line indicates timepoint when mice were taken off of treatment. P values in c-f were calculated using two-tailed, unpaired Student's t-test, and those in h and i calculated using log-rank test. Error bars, mean±SEM. **** P<0.0001, *** P<0.001, ** P<0.01, * P<0.05. n.s., not significant.

FIGS. 5A-J. EZH2 suppression reinstates SASP-associated chemokines to drive NK and T cell accumulation in PDAC. a, KEGG pathway analysis of RNA-seq data showing top enriched pathways in shEzh2 as compared to shRen KPC1 orthotopic PDAC tumor cells in mice treated with combined trametinib (1 mg/kg) and palbociclib (100 mg/kg) for 2 weeks (n=5-6 per group). b, Heatmap of RNA-seq analysis of SASP gene expression in tumor cells sorted from KPC1 orthotopic PDAC tumors harboring indicated shRNAs and treated with vehicle or combined trametinib (1 mg/kg) and palbociclib (100 mg/kg) for 2 weeks (n=5-6 per group). c, qRT-PCR analysis of Ccl2 expression in KPC1 PDAC cells engineered to overexpress (O/E) a Ccl2 cDNA or Empty control vector (n=3). A.U., arbitrary units. d, NK cell migration assay in the presence of conditioned media from KPC1 PDAC cells engineered to overexpress Ccl2 or an Empty control vector and treated with vehicle or trametinib (25 nM) and palbociclib (500 nM) for 8 days (n=3). e, Flow cytometry analysis of NK cell numbers in KPC1 orthotopic PDAC tumors expressing control Empty or Ccl2 vectors following treatment as in (b) (n≥4 per group). f, Kaplan-Meier survival curve of mice with KPC1 orthotopic PDAC tumors expressing control Empty (left) or Ccl2 (right) vectors treated with vehicle, combined trametinib (1 mg/kg) and palbociclib (100 mg/kg), and/or an NK1.1 depleting antibody (PK136; 250 μg) (n≥10 per group). g, Flow cytometry analysis of NK cell numbers in shEzh2 KPC1 orthotopic PDAC tumors following treatment with vehicle, combined trametinib (1 mg/kg) and palbociclib (100 mg/kg), and/or a CCL2 depleting antibody (2H5; 200 μg) for 2 weeks (n≥3 per group). h, Waterfall plot of the response of shEzh2 KPC1 orthotopic PDAC tumors to treatment as in (g) (n≥22). i, Flow cytometry analysis of CD4⁺ and CD8⁺ T cell numbers in shEzh2 KPC1 orthotopic PDAC tumors following treatment with vehicle, combined trametinib (1 mg/kg) and palbociclib (100 mg/kg), and/or a CXCR3 depleting antibody (CXCR3-173; 200 μg) for 2 weeks (n≥5 per group). j, Waterfall plot of the response of shEzh2 KPC1 orthotopic PDAC tumors to treatment as in (i) (n≥7 per group). P values in c-e and g-j were calculated using two-tailed, unpaired Student's t-test, and those in f calculated using log-rank test. Error bars, mean±SEM. **** P<0.0001, *** P<0.001, ** P<0.01, * P<0.05. n.s., not significant.

FIGS. 6A-H. Pharmacological EZH2 methyltransferase inhibition in combination with T/P reactivates cytotoxic NK and T cell immunity and enhances tumor control in preclinical PDAC models. a, Schematic of KPC PDAC syngeneic orthotopic transplant model and treatment regimens. b, Immunohistochemical (IHC) staining of KPC1 orthotopic PDAC tumors treated vehicle, trametinib (1 mg/kg) and palbociclib (100 mg/kg), and/or tazemetostat (Taz) (125 mg/kg) for 2 weeks. Scale bars, 50 μm. c, Waterfall plot of the response of KPC1 orthotopic PDAC tumors following 2 week-treatment with vehicle, trametinib (1 mg/kg) and palbociclib (100 mg/kg), and/or low (125 mg/kg) or high (400 mg/kg) doses of tazemetostat (n≥7 per group). d-e, Flow cytometry analysis of NK (d) and T cell (e) numbers and activation markers in KPC1 orthotopic PDAC tumors following treatment as in (c) (n≥7 per group). f, Waterfall plot of the response of KPC GEMM tumors to treatment as in (b) (n≥7 per group). g, IHC staining of KPC GEMM tumors treated as in (b). Scale bars, 50 μm. h, Quantification of NKp46⁺ NK cells, CD3⁺ T cells, and GZMB⁺ cells in (g) (n≥3 per group). P values in c-f and h were calculated using two-tailed, unpaired Student's t-test. Error bars, mean±SEM. **** P<0.0001, *** P<0.001, ** P<0.01, * P<0.05. n.s., not significant.

FIGS. 7A-E. EZH2 is associated with suppression of inflammatory chemokine signaling, reduced NK and T cell immune surveillance, and poor survival in PDAC patients. a, Pearson's correlation analysis plots comparing EZH2 and PRC2 repression signatures with inflammatory response gene sets, CCL2, CXCL9, and CXCL10 expression, and NK and CD8⁺ T cell signatures in human primary PDAC transcriptomic data⁵⁸ (n=145 samples). b, Representative IHC staining of surgically resected human PDAC tumors (n=30). Arrows indicate NK cells. Scale bars, 50 μm. c, Scoring of EZH2 and NKp46 expression from IHC staining in (b) (n=30). Percentage of samples with indicated EZH2 and NKp46 scores are shown, with the total number of samples in parentheses. d, Kaplan-Meier survival curve of human PDAC patients stratified based on EZH2 expression levels in (b) (n=9, 8, and 5 for EZH2 Lo, Int, and Hi, respectively). e, Kaplan-Meier survival curve of human PDAC patients stratified based on NKp46 expression levels in (b) (n=10 and 12 for NKp46 Lo and Hi/Int, respectively). P values in a were calculated using two-tailed, unpaired Student's t-test, and those in d and e were calculated using log-rank test. Error bars, mean±SEM. **** P<0.0001, *** P<0.001, ** P<0.01, * P<0.05. n.s., not significant.

FIGS. 8A-F. EZH2 knockdown in the KPC2 PDAC orthotopic transplant model also potentiates anti-tumor NK and CD8⁺ T cell immunity and long-term tumor regressions following T/P treatment. a, IHC staining of KPC1 (left) or KPC2 (right) orthotopic PDAC tumors harboring shRen or shEzh2 shRNAs treated with vehicle or combined trametinib (1 mg/kg) and palbociclib (100 mg/kg) (T/P) for 2 weeks. Scale bars, 50 μm. b-c, Flow cytometry analysis of NK cell (b) and T cell (c) numbers and activation markers in KPC2 orthotopic PDAC tumors harboring indicated shRNAs treated as in (a) (n≥5 per group). d, Flow cytometry analysis of F4/80⁺ macrophages in KPC1 orthotopic PDAC tumors harboring indicated shRNAs treated as in (a) (n≥6 per group). e, Waterfall plot of the response of KPC2 orthotopic PDAC tumors harboring indicated shRNAs to treatment as in (a) (n≥7 per group). f, Kaplan-Meier survival curve of mice with shEzh2 KPC2 orthotopic PDAC tumors treated with vehicle, combined trametinib (1 mg/kg) and palbociclib (100 mg/kg), and/or depleting antibodies against NK1.1 (PK136; 250 μg) or CD8 (2.43; 200 μg) (n≥5 per group). P values in b-e were calculated using two-tailed, unpaired Student's t-test, and those in f were calculated using log-rank test. Error bars, mean±SEM. **** P<0.0001, *** P<0.001, ** P<0.01, * P<0.05. n.s., not significant.

FIGS. 9A-E. EZH2 blockade reduces T/P-induced blood vessel formation and promotes CCL2 and CXCL9/10 secretion that increases NK and CD8⁺ T cell infiltration into PDAC. a, IHC staining of KPC1 orthotopic PDAC tumors harboring shRen or shEzh2 shRNAs treated with vehicle or combined trametinib (1 mg/kg) and palbociclib (100 mg/kg) (T/P) for 2 weeks. Scale bars, 50 μm. Quantification of blood vessels per field are shown on right (n=2-4). b-c, Flow cytometry analysis of NK cell activation markers (b) and CD4⁺ and CD8⁺ T cell numbers (c) in KPC1 orthotopic PDAC tumors expressing control Empty or Ccl2 vectors and treated as in (a). (n≥2 per group). d, Flow cytometry analysis of CD4⁺ and CD8⁺ T cell numbers in shEzh2 KPC1 orthotopic PDAC tumors following treatment with vehicle, combined trametinib (1 mg/kg) and palbociclib (100 mg/kg), and/or a CCL2 depleting antibody (2H5; 200 μg) for 2 weeks (n≥3 per group). e, Flow cytometry analysis of NK cell numbers in shEzh2 KPC1 orthotopic PDAC tumors following treatment with vehicle, combined trametinib (1 mg/kg) and palbociclib (100 mg/kg), and/or a CXCR3 depleting antibody (CXCR3-173; 200 μg) for 2 weeks (n≥5 per group). P values in a-e were calculated using two-tailed, unpaired Student's t-test. Error bars, mean±SEM. **** P<0.0001, *** P<0.001, ** P<0.01, * P<0.05. n.s., not significant.

FIGS. 10A-B. SMA⁺ fibroblast depletion leads to increased NK and T cells numbers and activation status following T/P-induced senescence. a, Schematic of KPC PDAC syngeneic orthotopic transplant model in SMA-TK mice and treatment regimens. Ganciclovir (GCV) treatment leads to selective depletion of SMA⁺ fibroblasts expressing SMA-TK allele. b, Flow cytometry analysis of NK and CD4⁺ and CD8⁺ T cells and their activation markers in transplanted KPC1 PDAC tumors in SMA-TK mice treated with vehicle, combined trametinib (1 mg/kg) and palbociclib (100 mg/kg), and/or GCV (50 mg/kg) for 2 weeks (n≥8 per group).

FIGS. 11A-B. SMA⁺ fibroblast depletion leads to increased inflammatory signaling and SASP induction following T/P treatment. SMA-TK mice bearing KPC1 transplant PDAC tumors were treated with vehicle, combined trametinib (1 mg/kg) and palbociclib (100 mg/kg), and/or GCV (50 mg/kg) for 2 weeks. GFP⁺ tumor cells were FACS sorted and extracted RNA subjected to RNA-seq analysis (n=4-5 per group). a, KEGG and REACTOME pathway analysis of pathways enriched in tumors treated with T/P+GCV compared to T/P alone are shown. b, Heatmap showing fold change in SASP gene expression following indicated treatments.

FIG. 12. SMA⁺ fibroblast depletion reverses EZH2 mediated target suppression following T/P treatment. SMA-TK mice bearing KPC1 transplant PDAC tumors were treated with vehicle, combined trametinib (1 mg/kg) and palbociclib (100 mg/kg), and/or GCV (50 mg/kg) for 2 weeks. GFP⁺ tumor cells were FACS sorted and extracted RNA subjected to RNA-seq analysis (n=4-5 per group). Transcription factor enrichment analysis showing transcriptional regulators whose targets are enriched T/P vs. T/P+GCV treated tumors.

FIG. 13. Conditioned Media from pancreatic stellate cells suppresses SASP in PDAC cell lines in vitro. a, KPC1 PDAC tumor cells were cultured in normal (basal) media or conditioned media (CM) from proliferating pancreatic cancer stellate cells and treated with combined trametinib (25 nM) and palbociclib (500 nM) for 7 days (top). Bottom, qRT-PCR analysis of SASP gene expression in T/P treated tumor cells grown in basal or fibroblast CM.

### DETAILED DESCRIPTION

Pancreatic ductal adenocarcinoma (PDAC) is a devastating disease with few effective treatment options, and has quickly risen to become the 3^{rd} leading cause of cancer-related death¹. Conventional chemotherapy regimens have limited efficacy in PDAC, in part due to a fibrotic and desmoplastic tumor microenvironment (TME) that leads to vascular dysfunction and poor drug delivery and activity in tumors²⁻⁴. Immunotherapy regimens including chimeric antigen receptor (CAR) T cells and anti-PD-1 and CTLA-4 immune checkpoint blockade (ICB) therapies that have been effective in other aggressive, chemo-refractory tumors have been ineffective in PDAC because of widespread innate and adaptive immune suppression in the pancreas TME⁵⁻⁷. Indeed, an abundance of suppressive macrophage and myeloid populations, poor tumor immunogenicity, and a lack of cytotoxic Natural Killer (NK) and T cell infiltration contribute to the immunologically “cold” TME associated with PDAC and immunotherapy resistance⁸. Thus, new and innovative approaches are needed to target the multiple axes of immune suppression in PDAC to achieve durable therapeutic outcomes.

Point mutations in KRAS are oncogenic drivers in PDAC and found in >90% of patients⁹. One strategy to increase tumor immunogenicity and stimulate anti-tumor immunity is to target the oncogenic pathways that drive immune suppression^{10,11}, including RAS signaling itself^{12,13}. RAS pathway targeting therapies have been shown not only to increase tumor immunogenicity through upregulating antigen presentation and processing genes (e.g. major histocompatibility complex (MHC) Class I (MHC-I) molecules) but also lead to immune stimulatory microenvironments that activate anti-tumor NK and T cell immunity and ICB therapy efficacy¹⁴⁻¹⁸. The combination of the MEK inhibitor trametinib and CDK4/6 inhibitor palbociclib (T/P) could trigger KRAS mutant cancers to enter cellular senescence, a stable cell cycle arrest program that is accompanied by a secretory program that can modulate immune responses (see US20210205319 and^{19,20}). This senescence-associated secretory phenotype (SASP) includes a collection of pleiotropic factors such as pro- and anti-inflammatory chemokines and cytokines, angiogenic factors, growth and stemness components, matrix metalloproteinases (MMPs), and lipid species that remodel the surrounding TME in both tumor promoting and tumor suppressive ways depending on the context²¹⁻²³.

In certain tumor and cancer therapy contexts, the SASP can mediate potent anti-tumor immunity to block tumor formation, regress established tumors, and enhance immunotherapy regimens^{19,20,24-27}. Therapy-induced senescence following T/P treatment can induce anti-tumor immune surveillance in preclinical mouse models of KRAS mutant lung adenocarcinoma (LUAD) and PDAC. In KRAS mutant LUAD, T/P-induced senescence led to secretion of pro-inflammatory SASP factors that activated NK cell immune surveillance and drove NK cell-mediated long-term lung tumor responses¹⁹. Intriguingly, in similar genetic models of KRAS mutant PDAC, T/P treatment led to a predominantly pro-angiogenic SASP that enhanced vascularization and CD8⁺ T cell extravasation into PDAC with little effect on NK cell immunity²⁰. Combining therapy-induced senescence with anti-PD-1 ICB enhanced cytotoxic T cell immunity and led to tumor responses in PDAC-bearing animals, demonstrating that the SASP could be a means to make “cold” PDAC tumors “hot” and potentiate currently ineffective ICB strategies.

A better understanding of why the SASP elicits altered immune responses in different cancer contexts and how the SASP transcriptional program or specific SASP factors can be optimized for immune-mediated tumor destruction²⁸ will help to harness senescence and its immune stimulating properties for tumor suppression. In the setting of PDAC, it will be important to elucidate how the pancreas TME suppresses cytotoxic NK cells that can act as potent eliminators of senescent tumor cells²⁹. As NK cells can both directly eradicate target cells through release of cytolytic granules, as well as indirectly mobilize adaptive T cell immunity through secretion of cytokines and chemokines, they are promising targets for cancer immunotherapy³⁰. The present study addressed why the SASP elicited different immune responses in the pancreas and how it could be harnessed for NK cell immunotherapy in PDAC.

As shown herein, enhancer of zeste 2 (EZH2) is induced in tumors by the pancreatic cancer microenvironment, and leads to the epigenetic repression of key pro-inflammatory SASP factors such as CCL2, CCL5, CCL8, CXCL9, CXCL10, IL-15, and IL-18 through its H3K27me3 methylating activity. The present results demonstrate that genetic or pharmacological inhibition of EZH2 activates these and other SASP factors necessary for NK cell immunity in pancreatic cancer, and can lead to NK cell-mediated tumor cures.

EZH2 is a member of the polycomb repressor complex 2 (PRC2) with methyltransferase activity that mediates transcriptional gene repression through H3K27 trimethylation (Laugesen et al., Cold Spring Harb. Perspect. Med. 6:a026575 (2016)). The other subunits of PRC2 are embryonic ectoderm development (EED), SUZ12 and RbAp48. Suppression of EZH2 has been shown to induce cellular senescence in fibroblasts through both activation of DNA-damage response pathways and loss of repressive H3K27me3 marks on CDKN2A, a canonical regulator of senescence-associated cell cycle arrest (Bracken et al., Genes Dev. 2007; 21:525-530; Ito et al., Cell Rep. 2018; 22:3480-3492; Shah et al., Genes Dev. 2013; 27(16):1787-1799). EZH2 has also been shown to repress chemokines such as CXCL9 and CXCL10 and inhibit T cell immunity and immunotherapy responses (Peng et al Nature 527, 249-253 (2015)). EZH2 inhibition leads to NK cell immunity in liver cancer by activating NK cell ligands on hepatocytes (Bugide et al., Proc. Natl. Acad. Sci. USA. 2018; 115:E3509-E3518).

EZH2 has been shown to facilitate tumor immune evasion and resistance to ICB therapy in other cancer settings⁴⁵⁻⁴⁸. Without wishing to be bound by theory, it is believed that EZH2 mediates PDAC immune suppression through inhibition of the pro-inflammatory transcriptome, secretome, and surfaceome associated with the SASP. Mechanistically, EZH2 methyltransferase activity was directly responsible for suppressing many SASP components, such that genetic or pharmacological inhibition of EZH2 in tumor cells triggered to senescence following T/P therapy led to a marked reduction in H3K37me3 marks and increased transcription of many SASP cytokines and chemokines. Clinically, EZH2 is commonly overexpressed in poorly differentiated PDAC and associated with chemoresistance^{60,61}. Analysis of patient samples in addition revealed that EZH2 was not only associated with suppression of inflammatory chemokines and NK and T cell immunity in the human disease, but also poor overall patient survival. Thus EZH2 is an important marker and inducer of immune suppression in PDAC that is therapeutically targetable.

The SASP is often considered a “double-edged sword” and can promote anti-tumor immune surveillance or alternatively pro-tumor immune evasion depending on the context^{21,22,28}. As shown herein, the resident tissue or TME context plays a key role in immune responses to senescence stimuli. Unexpectedly, observed a distinct SASP program induced in the pancreas compared to lung TME following T/P-induced senescence that contributed to a lack of NK cell immunity in PDAC. Whereas LUAD or PDAC tumors propagated in the lungs displayed induction of pro-inflammatory SASP factors (e.g. IL-6, IL-15, CCL2, CXCL9/10), tumors in the pancreas expressed high levels of pro-angiogenic SASP factors (e.g., VEGFs, PDGFs, MMPs). Increased EZH2 activity and H3K27me3 levels appeared to mediate this phenotypic switch, as EZH2 blockade led to induction of pro-inflammatory SASP while simultaneously reducing angiogenic SASP factor expression following T/P treatment. This study demonstrates that the quantity and quality of the SASP elicited following senescence induction is influenced in part by the TME and its impact on the epigenetic state of the cancer cell.

As shown herein, induction of chemokines through the SASP that drive NK and T cell trafficking into TMEs is a powerful approach to make immunologically “cold” tumors such as PDAC “hot”⁶². Remarkably, as shown herein, combining increased cytotoxic NK and CD8⁺ T cell trafficking via SASP chemokines with the enhanced immunogenicity of senescent cells was sufficient to potentiate anti-tumor immune surveillance in PDAC even in the absence of immune checkpoint blockade.

Tazemetostat and other EZH2 methyltransferase inhibitors have demonstrated efficacy and been implemented into the clinical care of hematological malignancies and sarcomas; however, they have yet to show potent activity as single agents in solid tumors⁷². The present findings provide rationale for combining EZH2 inhibitors with a senescence-inducing therapy—e.g., using a MEK and CDK4/6 inhibitor combination, or radiation and chemotherapy—to promote NK and T cell-mediated eradication of senescent PDAC lesions through pro-inflammatory SASP induction. The methods leverage EZH2 inhibitors as an immune oncology approach in combination with senescence-inducing agents to remodel the inflammatory tumor secretome for immune-mediated PDAC control.

The present findings demonstrate a link between EZH2, repression of pro-inflammatory SASP factors, and NK cell inhibition that is specific to pancreatic cancer. Thus, described herein is a therapeutic combination for use in treating pancreatic cancer. Though EZH2 inhibitors alone have failed in clinical trials for pancreatic cancer, the present methods include the administration of a combination of therapy-induced senescence with MEK and CDK4/6 inhibitors with EZH2 inhibitors, or inhibitors of other components of the PRC2 complex, as a therapy in subjects with PDAC.

Thus, described herein are compositions and methods for treating pancreatic cancer, comprising PRC2 inhibitors, MEK inhibitors, and CDK4/6 inhibitors.

### PRC2 Inhibitors

An inhibitor of PRC2 can inhibit any of the three core subunits of PRC2, i.e., EED, EZH2, or SUZ12; or RbAp48. In some embodiments, the inhibitor is an inhibitory nucleic acid. In some embodiments, the inhibitor is a small molecule that binds to and reduces activity of the subunit or of PRC2 as a whole, e.g., inhibitors of EZH2 methyltransferase activity, inhibitors that disrupt the protein-protein interactions among the PRC2 subunits, and inhibitors that trigger EZH2 degradation (e.g., by post-translational modification of EZH2). See, e.g., Table 2 of Duan et al., Journal of Hematology & Oncology. 13:104 (2020), which lists a number of EZH2 inhibitors and their mechanisms of action.

Small molecule inhibitors of EZH2 include, but are not limited to, inhibitors of EZH2 methyltransferase activity such as EPZ6438, GSK126 (GSK2816126), GSK343, GSK926, DZNep, Ell, EPZ005678, EPZ011989, CPI-1205, CPI-169, ZLD1039, PF-06821497, UNC1999, 3-deazaneplanocin A hydrochloride, SHR2554, EPZ-6438 (tazemetostat), and pyrrole-3-carboxamide derivatives carrying a pyridone fragment (Zhou et al., New J. Chem., 2020, 44, 2247-2255), Sinefungin, EI1 (most of the foregoing are S-adenosyl-methionine-competitive inhibitors); inhibitors of the EZH2-EED interaction of PRC2 such as stabilized alpha-helix of EZH2 (SAH-EZH2) peptides, Astemizole, MAK683/EED226, AZD9291 (Osimertinib, TAGRISSO); and inhibitors that trigger EZH2 degradation such as long non-coding RNA (lncRNA) ANCR, gambogenic acid (GNA) derivatives, e.g., GNA022. In some embodiments, the inhibitor of EZH2 is an S-adenosyl-methionine-competitive inhibitors of EZH2 methyltransferase activity with a 2-pyridone core, or stabilized alpha-helix of EZH2 (SAH-EZH2) peptides. See, e.g., Duan et al., Journal of Hematology & Oncology. 13:104 (2020); Singh, European Journal of Medicinal Chemistry, 15 Mar. 2019, 166:351-368.

In some embodiments, the inhibitor of EZH2 is not AZD9291 (Osimertinib, TAGRISSO).

Inhibitors of EED include MAK683 (US20160176882), LG1980 (Li et al., Theranostics 2021; 11(14):6873-689), EED226 (Qi et al., Nature Chemical Biology volume 13, pages 381-388 (2017)), EED210, EED666, EED162, EED709, and EED396 (Li et al., PLoS ONE 12(1): e0169855 (2017)), BR-001 (Dong et al., Cancer Res Nov. 1 2019 (79) (21) 5587-5596), and A-395 (He et al., Nat. Chem. Biol. 13, 389-395); others are described in Read et al., ACS Chem. Biol. 2019, 14, 10, 2134-2140.

### MEK Inhibitors

The mitogen-activated protein kinase (MAPK) signaling pathway plays critical roles in the regulation of diverse cellular activities, including cell proliferation, survival, differentiation, and motility (Karin, L. C. M. Nature, 410, 37-40 (2001)). Dysregulation of the MAPK pathway occurs in more than one-third of all malignancies. The classical MAPK pathway consists of Ras (a family of related proteins which is expressed in all animal cell lineages and organs), Raf (a family of three serine/threonine-specific protein kinases that are related to retroviral oncogenes), MEK (mitogen-activated protein kinase kinase), and ERK (extracellular signal-regulated kinases), sequentially relaying proliferative signals generated at the cell surface receptors into the nucleus through cytoplasmic signaling. MEK inhibitors target the Ras/Raf/MEK/ERK signaling pathway, inhibiting cell proliferation and inducing apoptosis.

Examples of small molecule MEK inhibitors include trametinib, cobimetinib, binimetinib, selumetinib, PD-325901, TAK-733, CL-1040 (PD184352), PD035901, MEK162, AZD8330, GDC-0623, refametinib, pimasertib, R04987655, R05126766, WX-554, HL-085, CInQ-03, G-573, PD184161, PD318088, PD98059, R05068760, U0126, and SL327. Examples of CDK4/6 inhibitors include palbociclib, ribociclib, and abemaciclib. The properties, efficacy, and therapeutic indications of the various MEK inhibitors are described in Cheng & Tian, Molecules 22, 1551 (2017). Exemplary structures of some of the MEK inhibitors disclosed herein are shown in FIG. 19 of US20210205319. Inhibitory nucleic acids can also be used.

### CDK4 CDK6 Inhibitors

CDK4 and CDK6 are cyclin-dependent kinases that control the transition between the G1 and S phases of the cell cycle. The S phase is the period during which the cell synthesizes new DNA and prepares itself to divide during mitosis. CDK4/6 activity is typically deregulated and overactive in cancer cells. Some cancers exhibit amplification or overexpression of the genes encoding cyclins or the CDKs themselves.

A major target of CDK4 and CDK6 during cell-cycle progression is the retinoblastoma protein (RB). When RB is phosphorylated, its tumor-suppressive properties are inactivated. Selective CDK4/6 inhibitors deactivate CDK4 and CDK6 and dephosphorylate RB, resulting in cell-cycle arrest. In some cases, the arrested cells enter a state of senescence. Examples of small molecule CDK4/6 inhibitors include the clinically-approved palbociclib, ribociclib, and abemaciclib, as well as SHR6390, Trilaciclib, Lerociclib, PROTAC9, PROTAC10, PROTAC BSJ-03-123, PROTAC 12, PROTAC 13, BSJ-02-162, BSJ-03-204, BSJ-01-187, BSJ-04-132, BSJ-01-184, and PROTAC 19 (see Adon et al., RSC Adv., 2021, 11, 29227-29246). 1-H-pyrazole-3-carboxamide derivatives, 4-thiazol-N-(pyridin-2-yl)pyrimidin-2-amine derivatives, pyrido[2,3-d]pyrimidine derivatives, imidazo[1′,2′:1,6]pyrido(2,3-d)pyrimidine derivatives, 4,5-dihydro-1H-pyrazolo[4,3-h]quinazoline derivatives, methoxybenzyl 5-nitroacridone derivatives, carbocycle-indole conjugates and oxindole-indole conjugates, are described in Adon et al., RSC Adv., 2021, 11, 29227-29246. Inhibitory nucleic acids can also be used.

### Inhibitory Nucleic Acids

Inhibitory nucleic acids useful in the present methods and compositions include antisense oligonucleotides, ribozymes, siRNA compounds, single- or double-stranded RNA interference (RNAi) compounds such as siRNA compounds, modified bases/locked nucleic acids (LNAs), peptide nucleic acids (PNAs), and other oligomeric compounds or oligonucleotide mimetics that hybridize to at least a portion of the target nucleic acid and modulate its function. In some embodiments, the inhibitory nucleic acids include antisense RNA, antisense DNA, chimeric antisense oligonucleotides, antisense oligonucleotides comprising modified linkages, interference RNA (RNAi), short interfering RNA (siRNA); or a short, hairpin RNA (shRNA), or combinations thereof. See, e.g., WO 2010040112.

Exemplary target nucleic acid sequences are shown in the following table.

| Target | GenBank Sequence Acc. No. |
|---|---|
| enhancer of | NM_004456.5 (isoform a) |
| zeste 2 (EZH2) | NM_152998.3 (isoform b) |
| | NM_001203247.2 (isoform c) |
| | NM_001203248.2 (isoform d) |
| | NM_001203249.2 (isoform e) |
| embryonic ectoderm | NM_003797.5 (isoform a) |
| development (EED) | NM_001308007.2 (isoform c) |
| | NM_001330334.2 (isoform d) |
| SUZ12 | NM_015355.4 (isoform 1) |
| | NM_001321207.2 (isoform 2) |
| RbAp48 also known as | NM_005610.3 (isoform a) |
| RBBP4 (RB binding protein 4, | NM_001135255.2 (isoform b) |
| chromatin remodeling factor) | NM_001135256.2 (isoform c) |
| mitogen-activated protein | NM_001297555.2 (isoform 1) |
| kinase kinase (MEK) | |
| also known as MAP2K7 (mitogen- | NM_001297556.2 (isoform 2) |
| activated protein kinase kinase 7) | NM_145185.4 (isoform 3) |
| cyclin-dependent kinase 4 (CDK4) | NM_000075.4 |
| cyclin dependent kinase 6 (CDK6) | NM_001259.8 (variant 1) |
| | NM_001145306.2 (variant 2) |

In some embodiments, the inhibitory nucleic acids are 10 to 50, 10 to 20, 10 to 25, 13 to 50, or 13 to 30 nucleotides in length. One having ordinary skill in the art will appreciate that this embodies inhibitory nucleic acids having complementary portions of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or any range therewithin. In some embodiments, the inhibitory nucleic acids are 15 nucleotides in length. In some embodiments, the inhibitory nucleic acids are 12 or 13 to 20, 25, or 30 nucleotides in length. One having ordinary skill in the art will appreciate that this embodies inhibitory nucleic acids having complementary portions of 12, 13, 14, 15, 16, 17, 18, 19, 20, 21, 22, 23, 24, 25, 26, 27, 28, 29 or 30 nucleotides in length, or any range therewithin (complementary portions refers to those portions of the inhibitory nucleic acids that are complementary to the target sequence).

The inhibitory nucleic acids useful in the present methods are sufficiently complementary to the target RNA, i.e., hybridize sufficiently well and with sufficient specificity, to give the desired effect. “Complementary” refers to the capacity for pairing, through hydrogen bonding, between two sequences comprising naturally or non-naturally occurring bases or analogs thereof. For example, if a base at one position of an inhibitory nucleic acid is capable of hydrogen bonding with a base at the corresponding position of a RNA, then the bases are considered to be complementary to each other at that position. 100% complementarity is not required.

Routine methods can be used to design an inhibitory nucleic acid that binds to the target sequence with sufficient specificity. In some embodiments, the methods include using bioinformatics methods known in the art to identify regions of secondary structure, e.g., one, two, or more stem-loop structures, or pseudoknots, and selecting those regions to target with an inhibitory nucleic acid. For example, “gene walk” methods can be used to optimize the inhibitory activity of the nucleic acid; for example, a series of oligonucleotides of 10-30 nucleotides spanning the length of a target RNA can be prepared, followed by testing for activity. Optionally, gaps, e.g., of 5-10 nucleotides or more, can be left between the target sequences to reduce the number of oligonucleotides synthesized and tested. GC content is preferably between about 30-60%. Contiguous runs of three or more Gs or Cs should be avoided where possible (for example, it may not be possible with very short (e.g., about 9-10 nt) oligonucleotides).

In some embodiments, the inhibitory nucleic acid molecules can be designed to target a specific region of the RNA sequence. For example, a specific functional region can be targeted, e.g., a region comprising a known RNA localization motif (i.e., a region complementary to the target nucleic acid on which the RNA acts). Alternatively or in addition, highly conserved regions can be targeted, e.g., regions identified by aligning sequences from disparate species such as primate (e.g., human) and rodent (e.g., mouse) and looking for regions with high degrees of identity. Percent identity can be determined routinely using basic local alignment search tools (BLAST programs) (Altschul et al., J. Mol. Biol., 1990, 215, 403-410; Zhang and Madden, Genome Res., 1997, 7, 649-656), e.g., using the default parameters.

Once one or more target regions, segments or sites have been identified, e.g., within a target sequence known in the art or provided herein, inhibitory nucleic acid compounds are chosen that are sufficiently complementary to the target, i.e., that hybridize sufficiently well and with sufficient specificity (i.e., do not substantially bind to other non-target RNAs), to give the desired effect.

In the context of this disclosure, hybridization means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. Complementary, as used herein, refers to the capacity for precise pairing between two nucleotides. For example, if a nucleotide at a certain position of an oligonucleotide is capable of hydrogen bonding with a nucleotide at the same position of a RNA molecule, then the inhibitory nucleic acid and the RNA are considered to be complementary to each other at that position. The inhibitory nucleic acids and the RNA are complementary to each other when a sufficient number of corresponding positions in each molecule are occupied by nucleotides which can hydrogen bond with each other. Thus, “specifically hybridisable” and “complementary” are terms which are used to indicate a sufficient degree of complementarity or precise pairing such that stable and specific binding occurs between the inhibitory nucleic acid and the RNA target. For example, if a base at one position of an inhibitory nucleic acid is capable of hydrogen bonding with a base at the corresponding position of a RNA, then the bases are considered to be complementary to each other at that position. 100% complementarity is not required.

It is understood in the art that a complementary nucleic acid sequence need not be 100% complementary to that of its target nucleic acid to be specifically hybridisable. A complementary nucleic acid sequence for purposes of the present methods is specifically hybridisable when binding of the sequence to the target RNA molecule interferes with the normal function of the target RNA to cause a loss of activity, and there is a sufficient degree of complementarity to avoid non-specific binding of the sequence to non-target RNA sequences under conditions in which specific binding is desired, e.g., under physiological conditions in the case of in vivo assays or therapeutic treatment, and in the case of in vitro assays, under conditions in which the assays are performed under suitable conditions of stringency. For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

In general, the inhibitory nucleic acids useful in the methods described herein have at least 80% sequence complementarity to a target region within the target nucleic acid, e.g., 90%, 95%, or 100% sequence complementarity to the target region within an RNA. For example, an antisense compound in which 18 of 20 nucleobases of the antisense oligonucleotide are complementary, and would therefore specifically hybridize, to a target region would represent 90 percent complementarity. Percent complementarity of an inhibitory nucleic acid with a region of a target nucleic acid can be determined routinely using basic local alignment search tools (BLAST programs) (Altschul et al., J. Mol. Biol., 1990, 215, 403-410; Zhang and Madden, Genome Res., 1997, 7, 649-656). Inhibitory nucleic acids that hybridize to an RNA can be identified through routine experimentation. In general the inhibitory nucleic acids must retain specificity for their target, i.e., must not directly bind to, or directly significantly affect expression levels of, transcripts other than the intended target.

For further disclosure regarding inhibitory nucleic acids, please see US2010/0317718 (antisense oligos); US2010/0249052 (double-stranded ribonucleic acid (dsRNA)); US2009/0181914 and US2010/0234451 (LNAs); US2007/0191294 (siRNA analogues); US2008/0249039 (modified siRNA); and WO2010/129746 and WO2010/040112 (inhibitory nucleic acids).

### Modified Inhibitory Nucleic Acids

In some embodiments, the inhibitory nucleic acids used in the methods described herein are modified, e.g., comprise one or more modified bonds or bases. A number of modified bases include phosphorothioate, methylphosphonate, peptide nucleic acids, or locked nucleic acid (LNA) molecules. Some inhibitory nucleic acids are fully modified, while others are chimeric and contain two or more chemically distinct regions, each made up of at least one nucleotide. These inhibitory nucleic acids typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. Chimeric inhibitory nucleic acids of the invention may be formed as composite structures of two or more oligonucleotides, modified oligonucleotides, oligonucleosides and/or oligonucleotide mimetics as described above. Such compounds have also been referred to in the art as hybrids or gapmers. In some embodiments, the oligonucleotide is a gapmer (contain a central stretch (gap) of DNA monomers sufficiently long to induce RNase H cleavage, flanked by blocks of LNA modified nucleotides; see, e.g., Stanton et al., Nucleic Acid Ther. 2012. 22: 344-359; Nowotny et al., Cell, 121:1005-1016, 2005; Kurreck, European Journal of Biochemistry 270:1628-1644, 2003; FLuiter et al., Mol Biosyst. 5(8):838-43, 2009). In some embodiments, the oligonucleotide is a mixmer (includes alternating short stretches of LNA and DNA; Naguibneva et al., Biomed Pharmacother. 2006 November; 60(9):633-8; Ørom et al., Gene. 2006 May 10; 372( ): 137-41). Representative United States patents that teach the preparation of such hybrid structures comprise, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference.

In some embodiments, the inhibitory nucleic acid comprises at least one nucleotide modified at the 2′ position of the sugar, most preferably a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrimidines, abasic residues or an inverted base at the 3′ end of the RNA. Such modifications are routinely incorporated into oligonucleotides and these oligonucleotides have been shown to have a higher Tm (i.e., higher target binding affinity) than; 2′-deoxyoligonucleotides against a given target.

A number of nucleotide and nucleoside modifications have been shown to make the oligonucleotide into which they are incorporated more resistant to nuclease digestion than the native oligodeoxynucleotide; these modified oligos survive intact for a longer time than unmodified oligonucleotides. Specific examples of modified oligonucleotides include those comprising modified backbones, for example, phosphorothioates, phosphotriesters, methyl phosphonates, short chain alkyl or cycloalkyl intersugar linkages or short chain heteroatomic or heterocyclic intersugar linkages. Most preferred are oligonucleotides with phosphorothioate backbones and those with heteroatom backbones, particularly CH2-NH—O—CH2, CH,˜N(CH3)˜O˜CH2 (known as a methylene(methylimino) or MMI backbone], CH2—O—N(CH3)-CH2, CH2-N(CH3)-N (CH3)-CH2 and O—N(CH3)-CH2-CH2 backbones, wherein the native phosphodiester backbone is represented as O—P—O—CH,); amide backbones (see De Mesmaeker et al. Ace. Chem. Res. 1995, 28:366-374); morpholino backbone structures (see Summerton and Weller, U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991.

Cyclohexenyl nucleic acid oligonucleotide mimetics are described in Wang et al., J. Am. Chem. Soc., 2000, 122, 8595-8602.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts; see U.S. Pat. Nos. 5,034,506; 5,166,315; 5,185,444; 5,214,134; 5,216,141; 5,235,033; 5,264,562; 5, 264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

One or more substituted sugar moieties can also be included, e.g., one of the following at the 2′ position: OH, SH, SCH₃, F, OCN, OCH₃ OCH₃, OCH₃ O(CH₂)n CH₃, O(CH₂)n NH₂ or O(CH₂)n CH₃ where n is from 1 to about 10; Ci to C10 lower alkyl, alkoxyalkoxy, substituted lower alkyl, alkaryl or aralkyl; Cl; Br; CN; CF3; OCF3; O—, S—, or N-alkyl; O—, S—, or N-alkenyl; SOCH3; SO2 CH3; ONO2; NO2; N3; NH2; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A preferred modification includes 2′-methoxyethoxy [2′-O—CH₂CH₂OCH₃, also known as 2′-O-(2-methoxyethyl)] (Martin et al, HeIv. Chim. Acta, 1995, 78, 486). Other preferred modifications include 2′-methoxy (2′-O—CH₃), 2′-propoxy (2′-OCH₂ CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group.

Inhibitory nucleic acids can also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine and 2,6-diaminopurine. Kornberg, A., DNA Replication, W. H. Freeman & Co., San Francisco, 1980, pp 75-77; Gebeyehu, G., et al. Nucl. Acids Res. 1987, 15:4513). A “universal” base known in the art, e.g., inosine, can also be included. 5-Me-C substitutions have been shown to increase nucleic acid duplex stability by 0.6-1.2<0>C. (Sanghvi, Y. S., in Crooke, S. T. and Lebleu, B., eds., Antisense Research and Applications, CRC Press, Boca Raton, 1993, pp. 276-278) and are presently preferred base substitutions.

It is not necessary for all positions in a given oligonucleotide to be uniformly modified, and in fact more than one of the aforementioned modifications may be incorporated in a single oligonucleotide or even at within a single nucleoside within an oligonucleotide.

In some embodiments, both a sugar and an internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds comprise, but are not limited to, U.S. Pat. Nos. 5,539,082; 5,714,331; and 5,719,262, each of which is herein incorporated by reference. Further teaching of PNA compounds can be found in Nielsen et al, Science, 1991, 254, 1497-1500.

Inhibitory nucleic acids can also include one or more nucleobase (often referred to in the art simply as “base”) modifications or substitutions. As used herein, “unmodified” or “natural” nucleobases comprise the purine bases adenine (A) and guanine (G), and the pyrimidine bases thymine (T), cytosine (C) and uracil (U). Modified nucleobases comprise other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine and 3-deazaguanine and 3-deazaadenine.

Further, nucleobases comprise those disclosed in U.S. Pat. No. 3,687,808, those disclosed in ‘The Concise Encyclopedia of Polymer Science And Engineering’, pages 858-859, Kroschwitz, J. I., ed. John Wiley & Sons, 1990, those disclosed by Englisch et al., Angewandle Chemie, International Edition', 1991, 30, page 613, and those disclosed by Sanghvi, Y. S., Chapter 15, Antisense Research and Applications', pages 289-302, Crooke, S. T. and Lebleu, B. ea., CRC Press, 1993. Certain of these nucleobases are particularly useful for increasing the binding affinity of the oligomeric compounds of the invention. These include 5-substituted pyrimidines, 6-azapyrimidines and N-2, N-6 and 0-6 substituted purines, comprising 2-aminopropyladenine, 5-propynyluracil and 5-propynylcytosine. 5-methylcytosine substitutions have been shown to increase nucleic acid duplex stability by 0.6-1.2<0>C (Sanghvi, Y. S., Crooke, S. T. and Lebleu, B., eds, ‘Antisense Research and Applications’, CRC Press, Boca Raton, 1993, pp. 276-278) and are presently preferred base substitutions, even more particularly when combined with 2′-O-methoxyethyl sugar modifications. Modified nucleobases are described in U.S. Pat. No. 3,687,808, as well as U.S. Pat. Nos. 4,845,205; 5,130,302; 5,134,066; 5,175, 273; 5, 367,066; 5,432,272; 5,457,187; 5,459,255; 5,484,908; 5,502,177; 5,525,711; 5,552,540; 5,587,469; 5,596,091; 5,614,617; 5,750,692, and 5,681,941, each of which is herein incorporated by reference.

In some embodiments, the inhibitory nucleic acids are chemically linked to one or more moieties or conjugates that enhance the activity, cellular distribution, or cellular uptake of the oligonucleotide. Such moieties comprise but are not limited to, lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al, Ann. N. Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Mancharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-t oxycholesterol moiety (Crooke et al., J. Pharmacol. Exp. Ther., 1996, 277, 923-937). See also U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5, 245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

These moieties or conjugates can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism or excretion of the compounds of the present invention. Representative conjugate groups are disclosed in International Patent Application No. PCT/US92/09196, filed Oct. 23, 1992, and U.S. Pat. No. 6,287,860, which are incorporated herein by reference. Conjugate moieties include, but are not limited to, lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety. See, e.g., U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

### Locked Nucleic Acids (LNAs)

In some embodiments, the modified inhibitory nucleic acids used in the methods described herein comprise locked nucleic acid (LNA) molecules, e.g., including [alpha]-L-LNAs. LNAs comprise ribonucleic acid analogues wherein the ribose ring is “locked” by a methylene bridge between the 2′-oxygen and the 4′-carbon—i.e., oligonucleotides containing at least one LNA monomer, that is, one 2′-O,4′-C-methylene-β-D-ribofuranosyl nucleotide. LNA bases form standard Watson-Crick base pairs but the locked configuration increases the rate and stability of the basepairing reaction (Jepsen et al., Oligonucleotides, 14, 130-146 (2004)). LNAs also have increased affinity to base pair with RNA as compared to DNA. These properties render LNAs especially useful as probes for fluorescence in situ hybridization (FISH) and comparative genomic hybridization, as knockdown tools for miRNAs, and as antisense oligonucleotides to target mRNAs or other RNAs, e.g., RNAs as described herein.

The LNA molecules can include molecules comprising 10-30, e.g., 12-24, e.g., 12, 13, 14, 15, 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the RNA. The LNA molecules can be chemically synthesized using methods known in the art.

The LNA molecules can be designed using any method known in the art; a number of algorithms are known, and are commercially available (e.g., on the internet, for example at exiqon.com). See, e.g., You et al., Nuc. Acids. Res. 34:e60 (2006); McTigue et al., Biochemistry 43:5388-405 (2004); and Levin et al., Nuc. Acids. Res. 34:e142 (2006). For example, “gene walk” methods, similar to those used to design antisense oligos, can be used to optimize the inhibitory activity of the LNA; for example, a series of oligonucleotides of 10-30 nucleotides spanning the length of a target RNA can be prepared, followed by testing for activity. Optionally, gaps, e.g., of 5-10 nucleotides or more, can be left between the LNAs to reduce the number of oligonucleotides synthesized and tested. GC content is preferably between about 30-60%. General guidelines for designing LNAs are known in the art; for example, LNA sequences will bind very tightly to other LNA sequences, so it is preferable to avoid significant complementarity within an LNA. Contiguous runs of more than four LNA residues, should be avoided where possible (for example, it may not be possible with very short (e.g., about 9-10 nt) oligonucleotides). In some embodiments, the LNAs are xylo-LNAs.

For additional information regarding LNAs see U.S. Pat. Nos. 6,268,490; 6,734,291; 6,770,748; 6,794,499; 7,034,133; 7,053,207; 7,060,809; 7,084,125; and 7,572,582; and U.S. Pre-Grant Pub. Nos. 20100267018; 20100261175; and 20100035968; Koshkin et al. Tetrahedron 54, 3607-3630 (1998); Obika et al. Tetrahedron Lett. 39, 5401-5404 (1998); Jepsen et al., Oligonucleotides 14:130-146 (2004); Kauppinen et al., Drug Disc. Today 2(3):287-290 (2005); and Ponting et al., Cell 136(4):629-641 (2009), and references cited therein.

### Making and Using Inhibitory Nucleic Acids

The nucleic acid sequences used to practice the methods described herein, whether RNA, cDNA, genomic DNA, vectors, viruses or hybrids thereof, can be isolated from a variety of sources, genetically engineered, amplified, and/or expressed/generated recombinantly. Recombinant nucleic acid sequences can be individually isolated or cloned and tested for a desired activity. Any recombinant expression system can be used, including e.g. in vitro, bacterial, fungal, mammalian, yeast, insect or plant cell expression systems.

Nucleic acid sequences of the invention can be inserted into delivery vectors and expressed from transcription units within the vectors. The recombinant vectors can be DNA plasmids or viral vectors. Generation of the vector construct can be accomplished using any suitable genetic engineering techniques well known in the art, including, without limitation, the standard techniques of PCR, oligonucleotide synthesis, restriction endonuclease digestion, ligation, transformation, plasmid purification, and DNA sequencing, for example as described in Sambrook et al. Molecular Cloning: A Laboratory Manual. (1989)), Coffin et al. (Retroviruses. (1997)) and “RNA Viruses: A Practical Approach” (Alan J. Cann, Ed., Oxford University Press, (2000)). As will be apparent to one of ordinary skill in the art, a variety of suitable vectors are available for transferring nucleic acids of the invention into cells. The selection of an appropriate vector to deliver nucleic acids and optimization of the conditions for insertion of the selected expression vector into the cell, are within the scope of one of ordinary skill in the art without the need for undue experimentation. Viral vectors comprise a nucleotide sequence having sequences for the production of recombinant virus in a packaging cell. Viral vectors expressing nucleic acids of the invention can be constructed based on viral backbones including, but not limited to, a retrovirus, lentivirus, adenovirus, adeno-associated virus, pox virus or alphavirus. The recombinant vectors capable of expressing the nucleic acids of the invention can be delivered as described herein, and persist in target cells (e.g., stable transformants).

Nucleic acid sequences used to practice this invention can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Nucleic acid sequences of the invention can be stabilized against nucleolytic degradation such as by the incorporation of a modification, e.g., a nucleotide modification. For example, nucleic acid sequences of the invention includes a phosphorothioate at least the first, second, or third internucleotide linkage at the 5′ or 3′ end of the nucleotide sequence. As another example, the nucleic acid sequence can include a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). As another example, the nucleic acid sequence can include at least one 2′-O-methyl-modified nucleotide, and in some embodiments, all of the nucleotides include a 2′-O-methyl modification. In some embodiments, the nucleic acids are “locked,” i.e., comprise nucleic acid analogues in which the ribose ring is “locked” by a methylene bridge connecting the 2′-0 atom and the 4′-C atom (see, e.g., Kaupinnen et al., Drug Disc. Today 2(3):287-290 (2005); Koshkin et al., J. Am. Chem. Soc., 120(50):13252-13253 (1998)). For additional modifications see US 20100004320, US 20090298916, and US 20090143326.

Techniques for the manipulation of nucleic acids used to practice this invention, such as, e.g., subcloning, labeling probes (e.g., random-primer labeling using Klenow polymerase, nick translation, amplification), sequencing, hybridization and the like are well described in the scientific and patent literature, see, e.g., Sambrook et al., Molecular Cloning; A Laboratory Manual 3 d ed. (2001); Current Protocols in Molecular Biology, Ausubel et al., eds. (John Wiley & Sons, Inc., New York 2010); Kriegler, Gene Transfer and Expression: A Laboratory Manual (1990); Laboratory Techniques In Biochemistry And Molecular Biology: Hybridization With Nucleic Acid Probes, Part I. Theory and Nucleic Acid Preparation, Tijssen, ed. Elsevier, N.Y. (1993).

### Pharmaceutical Compositions

Provided herein are pharmaceutical compositions that include at least one, two, or preferably all three of a PRC2 inhibitor (e.g., EZH2 or EED inhibitor), a MEK inhibitor, and a CDK4/6 inhibitor. The pharmaceutical compositions described herein can be manufactured by methods well known in the art such as conventional granulating, mixing, dissolving, encapsulating, lyophilizing, or emulsifying processes, among others. Compositions may be produced in various forms, including granules, precipitates, or particulates, powders, including freeze dried, rotary dried or spray dried powders, amorphous powders, tablets, capsules, syrup, suppositories, injections, emulsions, elixirs, suspensions or solutions.

Formulations may optionally contain solvents, diluents, and other liquid vehicles, dispersion or suspension aids, surface active agents, pH modifiers, isotonic agents, thickening or emulsifying agents, stabilizers and preservatives, solid binders, lubricants and the like, as suited to the particular dosage form desired. In certain embodiments, the compositions disclosed herein are formulated for administration to a mammal, such as a human.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, cyclodextrins, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. Compositions formulated for parenteral administration may be injected by bolus injection or by timed push, or may be administered by continuous infusion.

In order to prolong the effect of a compound of the present disclosure, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents such as phosphates or carbonates.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings, release controlling coatings and other coatings well known in the pharmaceutical formulating art. They may optionally contain opacifying agents and can also be of a composition that they release the active ingredient(s) only, or in a certain part of the intestinal tract, optionally, in a delayed manner. Examples of embedding compositions that can be used include polymeric substances and waxes.

The active compounds can also be in micro-encapsulated form with one or more excipients as noted above. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or in a certain part of the intestinal tract, optionally, in a delayed manner. Examples of embedding compositions that can be used include polymeric substances and waxes.

### Methods of Treatment

Described herein are methods for preventing or treating pancreatic cancer in a subject in need thereof. The methods include administering to the subject an effective amount of each of (i) a PRC2 inhibitor (e.g., an EZH2 or EED inhibitor), (ii) a MEK inhibitor, and (iii) a CDK4/6 inhibitor. The pancreatic cancer may be an exocrine pancreatic cancer or an endocrine pancreatic cancer. Examples of pancreatic cancers include, but are not limited to PDAC, acinar cell carcinoma, solid pseudopapillary neoplasms, pancreatoblastoma, pancreatic neuroendocrine tumors (PNETs), gastrinomas, insulinomas, glucagonomas, somatostatinomas and VIPomas.

In preferred embodiments, the pancreatic cancer comprises a KRAS mutation such as G12D, G12V, G12C, G12R, G12A, G13D, Q61L, Q61H etc. In certain embodiments, the subject is human. Additionally or alternatively, in some embodiments, the subject is non-responsive to at least one prior line of cancer therapy such as chemotherapy or immunotherapy.

In some embodiments, the methods can also include administering at least one chemotherapeutic agent. Examples of chemotherapeutic agents include abraxane, capecitabine, erlotinib, fluorouracil (5-FU), gemcitabine, irinotecan, leucovorin, nab-paclitaxel, cisplatin, irinotecan, docetaxel, oxaliplatin, tipifarnib, everolimus, sunitinib, dovitinib, ruxolitinib, pegylated-hyaluronidase, pemetrexed, folinic acid, paclitaxel, MK2206, GDC-0449, IPI-926, gamma secretase/R04929097, M402, and LY293111.

In some embodiments, the methods can also include administering at least one immunotherapeutic agent. Examples of immunotherapeutic agents include immune checkpoint inhibitors (e.g., anti-CD137 (BMS-663513), anti-PD1 (e.g., Nivolumab, pembrolizumab/MK-3475, Pidilizumab (CT-011)), anti-PDL1 (e.g., BMS-936559, MPDL3280A), or anti-CTLA-4 (e.g., ipilumimab; see, e.g., Krüger et al., Histol Histopathol. 2007 June; 22(6):687-96; Eggermont et al., Semin Oncol. 2010 October; 37(5):455-9; Klinke D J., Mol Cancer. 2010 Sep. 15; 9:242; Alexandrescu et al., J Immunother. 2010 July-August; 33(6):570-90; Moschella et al., Ann N Y Acad Sci. 2010 April; 1194:169-78; Ganesan and Bakhshi, Natl Med J India. 2010 January-February; 23(1):21-7; Golovina and Vonderheide, Cancer J. 2010 July-August; 16(4):342-7), and other agents including sipuleucel-T, CRS-207, and GVAX. Monoclonal antibodies targeting cancer-associated antigens, such as 90Y-Clivatuzumab tetraxetan, trastuzumab, cixutumumab, ganitumab, demcizumab, cetuximab, nimotuzumab, and dalotuzumab, can also be used.

In some embodiments of the methods disclosed herein, the PRC2 inhibitor, MEK inhibitor, and CDK4/6 inhibitor are administered sequentially, simultaneously, or separately. In some embodiments, a single composition comprising all three is administered. In some embodiments, two compositions are administered, wherein the PRC2 inhibitor and MEK inhibitor are administered in a single composition and the CDK4/6 inhibitor is administered separately; the PRC2 inhibitor and CDK4/6 inhibitor are administered in a single composition and the MEK inhibitor is administered separately; or the CDK4/6 inhibitor and MEK inhibitor are administered in a single composition and the PRC2 inhibitor is administered separately. In some embodiments, three compositions are administered, i.e., the PRC2 inhibitor, the MEK inhibitor, and the CDK4/6 inhibitor are administered separately. The PRC2 inhibitor, MEK inhibitor, and/or the CDK4/6 inhibitor, or any combination thereof, may be administered orally, parenterally, by inhalation spray, intranasally, buccally, or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, intravenously, or subcutaneously. Formulations including any one, two, or all three of a PRC2 inhibitor, MEK inhibitor, and/or CDK4/6 inhibitor disclosed herein may be designed to be short-acting, fast-releasing, or long-acting. In other embodiments, compounds can be administered in a local rather than systemic means, such as administration (e.g., by injection) at a tumor site.

In some embodiments, administration of the PRC2 inhibitor, MEK inhibitor, and/or CDK4/6 inhibitor can be separated in time (e.g., by 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), simultaneously with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) each other. In some embodiments, the MEK inhibitor is administered before the CDK4/6 inhibitor to a patient with pancreatic cancer.

In some embodiments, the MEK inhibitor and CDK4/6 inhibitor are administered to a patient, for example, a mammal, such as a human, in a sequence and within a time interval such that the inhibitor that is administered first acts together with the inhibitor that is administered second to provide greater benefit than if each inhibitor were administered alone. For example, the MEK inhibitor and CDK4/6 inhibitor can be administered at the same time or sequentially in any order at different points in time; however, if not administered at the same time, the MEK inhibitor and CDK4/6 inhibitor are administered sufficiently close in time so as to provide the desired therapeutic or prophylactic effect of the combination of the two inhibitors. In one embodiment, the MEK inhibitor and CDK4/6 inhibitor exert their effects at times which overlap. In some embodiments, the MEK inhibitor and CDK4/6 inhibitor each are administered as separate dosage forms, in any appropriate form and by any suitable route. In other embodiments, the MEK inhibitor and CDK4/6 inhibitor are administered simultaneously in a single dosage form.

It will be appreciated that the frequency with which any of these therapeutic agents can be administered can be once or more than once over a period of about 2 days, about 3 days, about 4 days, about 5 days, about 6 days, about 7 days, about 8 days, about 9 days, about 10 days, about 11 days, about 12 days, about 13 days, about 14 days, about 20 days, about 28 days, about a week, about 2 weeks, about 3 weeks, about 4 weeks, about a month, about every 2 months, about every 3 months, about every 4 months, about every 5 months, about every 6 months, about every 7 months, about every 8 months, about every 9 months, about every 10 months, about every 11 months, about every year, about every 2 years, about every 3 years, about every 4 years, or about every 5 years.

For example, a PRC2 inhibitor, MEK inhibitor, or CDK4/6 inhibitor may be administered daily, weekly, biweekly, or monthly for a particular period of time. A PRC2 inhibitor, MEK inhibitor, or CDK4/6 inhibitor may be dosed daily over a 14 day time period, or twice daily over a seven day time period. A PRC2 inhibitor, MEK inhibitor, or CDK4/6 inhibitor may be administered daily for 7 days.

Alternatively, a PRC2 inhibitor, MEK inhibitor, or CDK4/6 inhibitor may be administered daily, weekly, biweekly, or monthly for a particular period of time followed by a particular period of non-treatment. In some embodiments, the PRC2 inhibitor, MEK inhibitor, or CDK4/6 inhibitor can be administered daily for 14 days followed by seven days of non-treatment, and repeated for two more cycles of daily administration for 14 days followed by seven days of non-treatment. In some embodiments, the PRC2 inhibitor, MEK inhibitor, or CDK4/6 inhibitor can be administered twice daily for seven days followed by 14 days of non-treatment, which may be repeated for one or two more cycles of twice daily administration for seven days followed by 14 days of non treatment.

In some embodiments, the PRC2 inhibitor, MEK inhibitor, or CDK4/6 inhibitor is administered daily over a period of 14 days. In another embodiment, the PRC2 inhibitor, MEK inhibitor, or CDK4/6 inhibitor is administered daily over a period of 12 days, or 11 days, or 10 days, or nine days, or eight days. In another embodiment, the PRC2 inhibitor, MEK inhibitor, or CDK4/6 inhibitor is administered daily over a period of seven days. In another embodiment, the PRC2 inhibitor, MEK inhibitor, or CDK4/6 inhibitor is administered daily over a period of six days, or five days, or four days, or three days.

In some embodiments, individual doses of the PRC2 inhibitor, MEK inhibitor, and the CDK4/6 inhibitor are administered within a time interval such that the three inhibitors can work together (e.g., within 1 hour, 2 hours, 4 hours, 6 hours, 12 hours, 24 hours, 48 hours, 72 hours, 96 hours, 5 days, 6 days, 1 week, or 2 weeks). In some embodiments, the treatment period during which the therapeutic agents are administered is then followed by a non-treatment period of a particular time duration, during which the therapeutic agents are not administered to the patient. This non-treatment period can then be followed by a series of subsequent treatment and non-treatment periods of the same or different frequencies for the same or different lengths of time. In some embodiments, the treatment and non-treatment periods are alternated. It will be understood that the period of treatment in cycling therapy may continue until the patient has achieved a complete response or a partial response, at which point the treatment may be stopped. Alternatively, the period of treatment in cycling therapy may continue until the patient has achieved a complete response or a partial response, at which point the period of treatment may continue for a particular number of cycles. In some embodiments, the length of the period of treatment may be a particular number of cycles, regardless of patient response. In some other embodiments, the length of the period of treatment may continue until the patient relapses.

In some embodiments, the PRC2 inhibitor, MEK inhibitor, and the CDK4/6 inhibitor are cyclically administered to a patient. Cycling therapy involves the administration of a first agent or combination of agents (e.g., PRC2 inhibitor, MEK inhibitor, and/or CDK4/6 inhibitor) for a period of time, followed by the administration of a second agent or combination of agents, and optionally a fourth agent (e.g., a fourth therapeutic agent such as an immunotherapeutic) for a period of time and repeating this sequential administration. Cycling therapy can reduce the development of resistance to one or more of the therapies, avoid or reduce the side effects of one of the therapies, and/or improve the efficacy of the treatment.

In some embodiments, the PRC2 inhibitor, MEK inhibitor, and CDK4/6 inhibitor each are administered at a dose and schedule typically used for that agent during monotherapy. In some embodiments, when the PRC2 inhibitor, MEK inhibitor, and CDK4/6 inhibitor are administered concomitantly, one or both of the agents can advantageously be administered at a lower dose than typically administered when the agent is used during monotherapy, such that the dose falls below the threshold that an adverse side effect is elicited.

The therapeutically effective amounts or suitable dosages of the PRC2 inhibitor, MEK inhibitor, and the CDK4/6 inhibitor in combination depends upon a number of factors, including the nature of the severity of the condition to be treated, the particular inhibitor, the route of administration and the age, weight, general health, and response of the individual patient. In certain embodiments, the suitable dose level is one that achieves a therapeutic response as measured by tumor regression or other standard measures of disease progression, progression free survival, or overall survival. In other embodiments, the suitable dose level is one that achieves this therapeutic response and also minimizes any side effects associated with the administration of the therapeutic agent.

Suitable daily dosages of PRC2 inhibitors, MEK inhibitors, and CDK/6 inhibitors can generally range, in single or divided or multiple doses, from about 10% to about 120% of the maximum tolerated dose as a single agent. In some embodiments, the suitable dosages of the inhibitors are from about 20% to about 100% of the maximum tolerated dose as a single agent. In other embodiments, the suitable dosages of the inhibitors are from about 25% to about 90% of the maximum tolerated dose as a single agent. In some embodiments, the suitable dosages of the inhibitors are from about 30% to about 80% of the maximum tolerated dose as a single agent. In other embodiments, the suitable dosages of the inhibitors are from about 40% to about 75% of the maximum tolerated dose as a single agent. In some embodiments, the suitable dosages of the inhibitors are from about 45% to about 60% of the maximum tolerated dose as a single agent. In other embodiments, suitable dosages of the inhibitors are about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, or about 120% of the maximum tolerated dose as a single agent.

Dosage, toxicity and therapeutic efficacy of any therapeutic agent can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are advantageous. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds may be within a range of circulating concentrations that include the ED50 with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a circulating plasma concentration range that includes the IC50 (z.e., the concentration of the test compound which achieves a half-maximal inhibition of symptoms) as determined in cell culture. Such information can be used to determine useful doses in humans accurately. Levels in plasma may be measured, for example, by high performance liquid chromatography.

Typically, an effective amount of the PRC2 inhibitors, MEK inhibitors, or CDK4/6 inhibitor, sufficient for achieving a therapeutic or prophylactic effect, may range from about 0.000001 mg per kilogram body weight per day to about 10,000 mg per kilogram body weight per day. Suitably, the dosage ranges are from about 0.0001 mg per kilogram body weight per day to about 100 mg per kilogram body weight per day. For example dosages can be 1 mg/kg body weight or 10 mg/kg body weight every day, every two days or every three days or within the range of 1-10 mg/kg every week, every two weeks or every three weeks. In one embodiment, a single dosage of PRC2 inhibitors, MEK inhibitors, or CDK4/6 inhibitors ranges from 0 001 10,000 micrograms per kg body weight. In some embodiments, PRC2 inhibitors, MEK inhibitors, or CDK4/6 inhibitor concentrations in a carrier range from 0.2 to 2000 micrograms per delivered milliliter. An exemplary treatment regime entails administration once per day or once a week. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regime.

In some embodiments, a therapeutically effective amount of a PRC2 inhibitors, MEK inhibitors, or CDK4/6 inhibitor may be defined as a concentration of the PRC2 inhibitors, MEK inhibitors, or CDK4/6 inhibitors at the target tissue of 10¹² to 10⁶ molar, e.g, approximately 10⁷ molar. This concentration may be delivered by systemic doses of 0.001 to 100 mg/kg or equivalent dose by body surface area. The schedule of doses would be optimized to maintain the therapeutic concentration at the target tissue, such as by single daily or weekly administration, but also including continuous administration (e.g., parenteral infusion or transdermal application).

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The mammal treated in accordance with the present methods can be any mammal, including, for example, farm animals, such as sheep, pigs, cows, and horses; pet animals, such as dogs and cats; laboratory animals, such as rats, mice and rabbits. In some embodiments, the mammal is a human.

### Kits

The present disclosure provides kits for treating pancreatic cancer comprising a PRC2 inhibitor, MEK inhibitor, and a CDK4/6 inhibitor, e.g., as disclosed herein, and instructions for treating pancreatic cancer. When simultaneous administration is contemplated, the kit may comprise a PRC2 inhibitor, MEK inhibitor, and a CDK4/6 inhibitor that has been formulated into a single pharmaceutical composition such as a tablet, or as separate pharmaceutical compositions. When the PRC2 inhibitor, MEK inhibitor, and CDK4/6 inhibitor are not administered simultaneously, the kit may comprise a PRC2 inhibitor, MEK inhibitor, and/or a CDK4/6 inhibitor that has been formulated as separate pharmaceutical compositions either in a single package, or in separate packages.

Additionally or alternatively, in some embodiments, the kits can further comprise at least one chemotherapeutic agent and/or at least one immunotherapeutic agent that are useful for treating pancreatic cancer, e.g., as known in the art and/or described herein.

The kits may further comprise pharmaceutically acceptable excipients, diluents, or carriers that are compatible with one or more kit components described herein. Optionally, the above described components of the kits of the present technology are packed in suitable containers and labeled for the treatment of pancreatic cancer. Examples of pancreatic cancers include, but are not limited to PDAC, acinar cell carcinoma, solid pseudopapillary neoplasms, pancreatoblastoma, pancreatic neuroendocrine tumors (PNETs), gastrinomas, insulinomas, glucagonomas, somatostatinomas and VIPomas. The kits may optionally include instructions customarily included in commercial packages of therapeutic products, that contain information about, for example, the indications, usage, dosage, manufacture, administration, contraindications and/or warnings concerning the use of such therapeutic products.

### EXAMPLES

The invention is further described in the following examples, which do not limit the scope of the invention described in the claims.

### Materials and Methods

The following materials and methods were used in the Examples below.

### Cell Lines and Compounds

PANC-1 cells were purchased from the American Type Culture Collection (ATCC). Murine PDAC (KPC1, KPC2) and LUAD (KP1, KP2) cell lines were generated as previously described^{19,20}. Pancreatic stellate cells from C57BL/6 mice and immortalized with TERT were a gift from Dr. Craig Thompson. For visualizing and tracking KPC and KP tumor cell lines with luciferase and GFP in vivo, cells were transduced with the following retroviral constructs: MSCV-luciferase (luc)-IRES-GFP (for KPC1 and KP1), MSCV-IRES-GFP (for KP2), and MSCV-shRen-PGK-Puro-IRES-GFP (for KPC2). Retroviruses were packaged by co-transfection of Gag-Pol expressing 293 T cells with expression constructs and envelope vectors (VSV-G). Following transduction, cells were purified by FACS sorting the GFP⁺ population on a FACSAria (BD Biosciences). All cells were maintained in a humidified incubator at 37° C. with 5% CO₂, and grown in DMEM supplemented with 10% FBS and 100 IU/ml penicillin/streptomycin (P/S). KPC cell lines were grown in culture dishes coated with 100 μg/ml collagen (PureCol) (5005; Advanced Biomatrix). All cell lines used were negative for mycoplasma. Human cell lines were authenticated by their source repository.

Trametinib (S2673), palbociclib (S1116), and GSK126 (S7061) were purchased from Selleck chemicals for in vitro studies. Drugs for in vitro studies were dissolved in DMSO (vehicle) to yield 10 mM stock solutions and stored at −80° C. For in vitro studies, growth media with or without drugs was changed every 2-3 days. For in vivo studies, trametinib (T-8123) and palbociclib (P-7744) were purchased from LC Laboratories, tazemetostat (HY-13803) from MedChemExpress, and GCV from InvivoGen. Trametinib was dissolved in a 0.5% hydroxypropyl methylcellulose and 0.2% Tween-80 solution, palbociclib in 50 mM sodium lactate buffer (pH 4), and tazemetostat in a 0.5% sodium carboxymethylcellulose and 0.1% Tween-80 solution (Sigma-Aldrich).

Short-Hairpin RNA (shRNA) Knockdown

shRNAs targeting Ezh2, Suz12, and Renilla (Ren) were cloned into the XhoI EcoRI locus of MLP retroviral vectors (MSCV-LTR-shRNA-PGK-Puro-IRES-GFP) as previously described⁷³. Retroviruses were packaged by co-transfection of Gag-Pol expressing 293 T cells with expression constructs and envelope vectors (VSV-G) using polyethylenimine (PEI; Sigma-Aldrich). Following transduction with shRNA retroviral constructs, cell selection was performed with 4 μg/ml puromycin for 1 week. Knockdown was confirmed by Western blot, qRT-PCR, and immunohistochemistry following transplantion into C57BL/6 mice.

### CCL2 Overexpression

Murine Ccl2 cDNA was cloned into an MSCV-based retroviral vector (MSCV-blast). Retroviruses were packaged by co-transfection of Gag-Pol expressing 293 T cells with expression constructs and envelope vectors (VSV-G) using polyethylenimine (PEI; Sigma-Aldrich). Following transduction with Ccl2 or control Empty constructs, cell selection was performed with 10 μg/ml Blasticidin S for 1 week. Ccl2 expression was confirmed by qRT-PCR.

### SA-β-gal Staining

SA-β-gal staining was performed as previously described at pH 5.5 for mouse cells and tissue^{19,20}. Fresh frozen sections of tumor tissue, or adherent cells plated in 6-well plates, were fixed with 0.5% glutaraldehyde in PBS for 15 min, washed with PBS supplemented with 1 mM MgCl₂, and stained for 4-18 hours in PBS containing 1 mM MgCl₂, 1 mg/ml X-Gal, and 5 mM each of potassium ferricyanide and potassium ferrocyanide. Tissue sections were counterstained with eosin. 5-10 high power 20× fields per tissue section were counted and averaged.

### Drug Withdrawal Clonogenic Assays

KPC tumor cells were initially plated in 6-well plates and pre-treated with vehicle (DMSO), trametinib (25 nM), palbociclib (500 nM), and/or tazemetostat (5 μM) for 8 days. Pre-treated cells were then trypsinized, and 5×103 cells re-plated per well of a 6-well plate in the absence of drugs for 7 days. Remaining cells were fixed with methanol (1%) and formaldehyde (1%), stained with 0.5% crystal violet, and photographed using a digital scanner.

### Immunoblotting

Cell lysis was performed using RIPA buffer (Cell Signaling) supplemented with phosphatase inhibitors (5 mM sodium fluoride, 1 mM sodium orthovanadate, 1 mM sodium pyrophosphate, 1 mM β-glycerophosphate) and protease inhibitors (Protease Inhibitor Cocktail Tablets, Roche). Protein concentration was determined using a Bradford Protein Assay kit (Biorad). Proteins were separated by SDS-PAGE and transferred to polyvinyl difluoride (PVDF) membranes (Millipore) according to standard protocols. Membranes were immunoblotted with antibodies against EZH2 (5246) and SUZ12 (3737) from Cell Signaling in 5% milk in TBS blocking buffer. After primary antibody incubation, membranes were probed with an ECL anti-rabbit IgG secondary antibody (1:10,000) from GE Healthcare Life Science and imaged using a ChemiDoc imaging system (BioRad). Protein loading was measured using a monoclonal β-actin antibody directly conjugated to horseradish peroxidase (A3854, Sigma-Aldrich) and imaged as above.

qRT-PCR

Total RNA was isolated using the RNeasy Mini Kit (Qiagen), and complementary DNA (cDNA) was obtained using the TaqMan reverse transcription reagents (Applied Biosystems). Real-time PCR was performed in triplicate using SYBR Green PCR Master Mix (Applied Biosystems) on the StepOnePlus Real-Time PCR system (Applied Biosystems). β-actin or Gapdh served as endogenous normalization controls. qRT-PCR primer sequences can be found in Table 1.

TABLE 1
qRT-PCR primer sequences

| | | |
|---|---|---|
| Gene | Sequence | SEQ ID NO: |

Mouse Primers

| | | |
|---|---|---|
| Ccl20 F | TTTTGGGATGGAATTGGACAC | 1. |
| Ccl20 R | TGCAGGTGAAGCCTTCAACC | 2. |
| Ccl7 F | TGAAAACCCCAACTCCAAAG | 3. |
| Ccl7 R | CATTCCTTAGGCGTGACCAT | 4. |
| Ccl8 F | CGCAGTGCTTCTTTGCCT | 5. |
| Ccl8 R | TCTGGCCCAGTCAGCTTCTC | 6. |
| Vegfa F | GCACATAGAGAGAATGAGCTTCC | 7. |
| Vegfa R | CTCCGCTCTGAACAAGGCT | 8. |
| Cxcl1 F | TCCAGAGCTTGAAGGTGTTGCC | 9. |
| Cxcl1 R | AACCAAGGGAGCTTCAGGGTC | 10. |
| Bactin F | GGCTGTATTCCCCTCCATCG | 11. |
| BactinR | CCAGTTGGTAACAATGCCATGT | 12. |
| Il6 F | TGATGCACTTGCAGAAAACA | 13. |
| Il6 R | ACCAGAGGAAATTTTCAATAGGC | 14. |
| Il15F | TGCCCTGAACTGCTTTCTCC | 15. |
| Il15 R | TCTCCTCCAGCTCCTCACAT | 16. |
| Il18 F | GAGGAAATGGATCCACCTGA | 17. |
| Il18 R | ATCTTCCTTTTGGCAAGCAA | 18. |
| Ccl2 F | CCTGCTGTTCACAGTTGCC | 19. |
| Ccl2 R | ATTGGGATCATCTTGCTGGT | 20. |
| Ccl4 F | GCCCTCTCTCTCCTCTTGCT | 21. |
| Ccl4 R | GTCTGCCTCTTTTGGTCAGG | 22. |
| Cxcl10 F | GTGAGAATGAGGGCCATAGG | 23. |
| Cxcl10 R | TTTTTGGCTAAACGCTTTCAT | 24. |
| Cxcl2 F | AGTGAACTGCGCTGTCAATG | 25. |
| Cxcl2 R | TTCAGGGTCAAGGCAAACTT | 26. |
| Icam1 F | CTGCTACCTGCACTTTGCC | 27. |
| Icam1 R | TGGATACCTGAGCATCACCA | 28. |
| Raet1d F | GGAAAGATGATGGGGAATTTG | 29. |
| Raet1d R | GAAGCGGGGAAGTTGATGTA | 30. |
| Raet1e F | GGAAAGATGATGGGGACCTT | 31. |
| Raet1e R | GAAGCGGGGAAGTTGATGTA | 32. |
| Cdkn2b F | AGTTGGGTTCTGCTCCGTGGAG | 33. |
| Cdkn2b R | ATCCCAACGCCCTGAACCGCT | 34. |
| Cdkn2a F | TGTTGAGGCTAGAGAGGATCTTG | 35. |
| Cdkn2a R | CGAATCTGCACCGTAGTTGAGC | 36. |
| Ccl5 F | GTGCCCACGTCAAGGAGTAT | 37. |
| Ccl5 R | CCACTTCTTCTCTGGGTTGG | 38. |
| Ulbp1 F | TCTCTGTCCTCGGAAGTTGAA | 39. |
| Ulbp1 R | GGGATCCCATCAATATCGTCT | 40. |
| H60a F | GAGCCACCAGCAAGAGCAA | 41. |
| H60a R | CCAGTATGGTCCCCAGATAGCT | 42. |
| H60b F | GGGTCTGAGTTTCACCTGGA | 43. |
| H60b R | CCCAGCACAGCTTGTTATGA | 44. |
| Gapdh F | GCAGTGGCAAAGTGGAGATT | 45. |
| Gapdh R | GAATTTGCCGTGAGTGGAGT | 46. |

Human Primers

| | | |
|---|---|---|
| IL15 F | AACAGAAGCCAACTGGGTGAATG | 47. |
| IL15 R | CTCCAAGAGAAAGCACTTCATTG | 48. |
| | C | |
| IL6 F | AGACAGCCACTCACCTCTTCAG | 49. |
| IL6 R | TTCTGCCAGTGCCTCTTTGCT | 50. |
| IL18 F | GATAGCCAGCCTAGAGGTATGG | 51. |
| IL18 R | CCTTGATGTTATCAGGAGGATTC | 52. |
| | A | |
| CXCL2 F | GGCAGAAAGCTTGTCTCAACCC | 53. |
| CXCL2 R | CTCCTTCAGGAACAGCCACCA | 54. |
| CXCL1 F | GCCCAAACCGAAGTCATAGCC | 55. |
| CXCL1 R | ATCCGCCAGCCTCTATCACA | 56. |
| CXCL10 F | GGTGAGAAGAGATGTCTGAATCC | 57. |
| CXCL10 R | GTCCATCCTTGGAAGCACTGCA | 58. |
| CCL8 F | TATCCAGAGGCTGGAGAGCTAC | 59. |
| CCL8R | TGGAATCCCTGACCCATCTCTC | 60. |
| CCL20 F | AAGTTGTCTGTGTGCGCAAATCC | 61. |
| CCL20 R | CCATTCCAGAAAAGCCACAGTTT | 62. |
| | T | |
| CCL4 F | GCTTCCTCGCAACTTTGTGGTAG | 63. |
| CCL4 R | GGTCATACACGTACTCCTGGAC | 64. |
| CCL7 F | ACAGAAGGACCACCAGTAGCCA | 65. |
| CCL7 R | GGTGCTTCATAAAGTCCTGGACC | 66. |
| CCL2 F | TTCTGTGCCTGCTGCTCATA | 67. |
| CCL2 R | AGCTTCTTTGGGACACTTGC | 68. |
| ICAM1 F | AGCTTCTCCTGCTCTGCAAC | 69. |
| ICAM1 R | ACACTTGAGCTCGGGCAAT | 70. |
| ULBP1 F | GGGAGAAGAACAGGGATGTG | 71. |
| ULBP1 R | ATGAGAAGGCTGCAGGGACT | 72. |
| ULBP2 F | TGGAAAGCACAGAACCCAGT | 73. |
| ULBP2 R | CTGCCCATCGAAACTGAACT | 74. |
| ULBP3 F | TAGCGGACTGACCACCTTCT | 75. |
| ULBP3 R | GGATGATGAGGAAGCTCCAG | 76. |
| ULBP4 F | GGCTCAGGGAATTCTTAGGG | 77. |
| ULBP4 R | CATTTTGCCACCAGACACAG | 78. |
| ULBP5 F | CCTCCCCTGCTTCATCCTAC | 79. |
| ULBP6 R | GCTGCGCCGTTATTTATTGT | 80. |
| MICA F | GCGATGAACGTCAGGAATTT | 81. |
| MICA R | CTGCATGTCACGGTGATGTT | 82. |
| MICB F | CTGGTGCTTCAGAGTCAACG | 83. |
| MICB R | CTGTGGTCTCCTGTCCCAAC | 84. |
| FAS F | GGAAAGCTAGGGACTGCACA | 85. |
| FAS R | CGGGTGCAGTTTATTTCCAC | 86. |
| CCL5 F | CTGCTGCTTTGCCTACCTCT | 87. |
| CCL5 R | CGAGTGACAAACACGACTGC | 88. |
| GAPDH F | GTCAGTGGTGGACCTGACCT | 89. |
| GAPDH R | TCGCTGTTGAACTCAGAGGA | 90. |

### CUT & Tag Analysis

CUT&Tag was performed largely as previously described⁴⁹. Trypsinized cells were counted, and 100,000 cells were washed and resuspended in wash buffer (20 mM HEPES pH 7.5; 150 mM NaCl; 0.5 mM Spermidine; 1× Protease inhibitor cocktail) and used for CUT&Tag. 10 μl of activated Concanavalin A coated magnetic beads (Polysciences) were added per sample and incubated at room temperature (RT) for 15 min. Bead-bound cells were resuspended in 100 μl Dig-wash Buffer (20 mM HEPES pH 7.5; 150 mM NaCl; 0.5 mM Spermidine; 0.05% Digitonin; 1× Protease inhibitor cocktail) containing 2 mM EDTA and 1 μl of H3K27me3 antibody (ThermoFisher, MA5-11198). The mixture was incubated overnight at 4° C. for antibodies to bind. After pulling beads to the side of the tube using a magnetic rack and removal of unbound primary antibody, beads were resuspended in 100 μl Dig-wash Buffer containing 1 μl of Guinea Pig anti-Rabbit antibody (Antibodies-Online, ABIN101961) and incubated for 30 mins at RT. Cells were washed 3 times with Dig-wash and then incubated with a 1:50 dilution of pA-Tn5 adapter complex in Dig-med (0.05% Digitonin, 20 mM HEPES, pH 7.5, 300 mM NaCl, 0.5 mM Spermidine, 1× Protease inhibitor cocktail) at RT for 1 hr. Cells were washed thrice in Dig-med Buffer and then resuspended in 300 μl Dig-med Buffer containing 10 mM MgCl₂ and incubated at 37° C. for 1 hr to activate tagmentation. To stop tagmentation, 10 μl of 0.5 M EDTA, 3 μl of 10% SDS and 1 μl of 20 mg/ml Proteinase K was added to each tube, which were incubated at 55° C. for 1 hr. DNA was extracted by performing one phenol:chloroform extraction followed by ethanol precipitation. The DNA pellet was resuspended in 22 μl of 10 mM Tris pH 8.

CUT&Tag libraries were amplified by mixing 21 μl of tagmented DNA with 2 μl each of (10 μM) barcoded i5 and i7 primers⁷⁴, using a different combination for each sample. 25 μl NEBNext HiFi 2× PCR Master mix (NEB) was added to each, and PCR was performed using the following cycling conditions: 72° C. for 5 min (gap filling); 98° C. for 30 s; 17 cycles of 98° C. for 10 s and 63° C. for 30 s; final extension at 72° C. for 1 min and holding at 4° C. 1.1× volumes of Ampure XP beads (Beckman Coulter) were incubated with libraries for 10 min at RT to clean up the PCR reaction. Bead bound DNA was purified by washing twice with 80% ethanol and eluting in 20 μl 10 mM Tris pH 8.0.

The libraries were quantified by Qubit and paired-end sequencing was performed on an Illumina NextSeq 500 (38 bases for reads 1 and 2 and 8 base indexing on both ends). Paired-end reads were aligned to the mouse reference genome GRCm38 (Ensembl, version 101) using bwa mem⁷⁵ after quality assurance with FastQC (bioinformatics.babraham.ac.uk/projects/fastqc/). Alignment files in the SAM format were first sorted by coordinates and converted into the BAM format using SAMtools⁷⁶. Subsequently, PCR duplicates were removed from the BAM files using “MarkDuplicates” command of the Picard tools (broadinstitute.github.io/picard/). The resulting BAM files were name sorted using SAMtools again. Peaks per condition were called using Genrich (github.com/jsh58/Genrich) with name-sorted, de-duplicated BAM files of all biological replicates for a given condition as input and a q-value cutoff of 0.05. Given that H3K27me3 modification are widespread across inactive gene regions, peaks with sizes less than 1 kb were filtered out. Consensus peaks-by-sample count matrix were determined using DiffBind⁷⁷. Differential peak analysis was conducted using DEseq2⁷⁸ with hidden variations adjusted for using svaseq⁷⁹. Peaks with absolute values of log₂ (shrunken fold change) greater than one and p-values less than 0.05, which were corrected for multiple testing using the Benjamini-Hochberg procedure⁸⁰, were considered as significantly differential peaks. Over-representation analysis of differential peaks against custom gene sets and each collection of the MSigDB gene sets^{81,82} was performed using msigdbr (github.com/igordot/msigdbr) and clusterProfiler⁸³. Track views were generated using the Integrative Genomics Viewer (IGV)⁸⁴.

### Cytokine Array

Cells were plated in duplicate or triplicate in 6-well plates and drug treated for 6 days. On day 6, 2 ml of new drug-containing media was added to each well and cells were incubated an additional 48 hours. Conditioned media was then collected and cells trypsinized and counted using a Countess II cell counter (Invitrogen). Media samples were then normalized based on cell number by diluting with culture media. Aliquots (75 μl) of the conditioned media were analyzed using a multiplex immunoassay (Mouse Cytokine/Chemokine 44-Plex array) from Eve Technologies.

### NK Cell Migration Assay

Primary NK cells were isolated and enriched the day of the experiment from the spleens of 8-12 week old female C57BL/6 mice using the NK Cell Isolation Kit II according to manufacturer's instructions (Miltenyi Biotec). 50,000 NK cells were then seeded in the top chamber of a transwell insert (Corning) in a 24-well dish in serum-Free DMEM media with 100 IU/ml penicillin/streptomycin. Serum-free conditioned media from KPC tumor cells (collected for 48 hrs following 6 day pre-treatment with indicated drugs) was then placed in the bottom chamber. Following 4 hr incubation in a 37° C. cell culture incubator, NK cells migrating through the bottom chamber were fixed with 4% paraformaldehyde (PFA), stained with DAPI, and counted on a Celigo imaging cytometer (Nexcelom).

### Animal Models

All mouse experiments were approved by the University of Massachusetts Chan Medical School Internal Animal Care and Use Committee. Mice were maintained under specific pathogen-free conditions, and food and water were provided ad libitum. C57BL/6 mice were purchased from Charles River and P48-Cre strains purchased from Jackson Laboratory. Trp53^{fl/fl} and Kras^{+/LSL-G12D} breeding pairs were generously provided by Wen Xue. SMA-TK mice were purchased from Jackson Laboratory.

### Pancreas Transplant Tumor Models

5×10⁴ KPC1, 2.5×10⁵ KPC2, 5×10⁴KP1, or 1×10⁵ KP2 cells were resuspended in 25 μl of Matrigel (Matrigel, BD) diluted 1:1 with cold PBS and transplanted into the pancreas of 8-10 week old C57BL/6 or SMA-TK female mice. Following anesthetization using 2-3% isoflurane, an incision was made in the left abdominal side and the cell suspension was injected into the tail region of the pancreas using a Hamilton Syringe. Successful injection was verified by the appearance of a fluid bubble without signs of intraperitoneal leakage. The abdominal wall was sutured with an absorbable Vicryl suture (Ethicon), and the skin was closed with wound clips (CellPoint Scientific Inc.). Mice were monitored for tumor development by ultrasound imaging, and randomized into treatment groups 1-week post-transplantation based on tumor volume. Upon sacrifice pancreas tumor tissue was allocated for 10% formalin fixation, OCT frozen blocks, flow cytometry analysis, and FACS sorting for downstream RNA-seq analysis.

### Lung Transplant Tumor Models

5×10⁵ KPC1, 5×10⁵ KPC2, 4×10⁴KP1, or 2.5×10⁵ KP2 cells were resuspended in PBS and transplanted by tail vein injection into 8-10 week old C57BL/6 female mice. Mice were monitored for tumor development by bioluminescence imaging (BLI) on a Xenogen IVIS (Caliper Life Sciences) and randomized into various treatment cohorts 1-week post-transplantation. Upon sacrifice lung lobes were allocated for 10% formalin fixation (1 lobe), OCT frozen blocks (1 lobe), and flow cytometry analysis and FACS sorting (3 lobes).

### Liver Transplant Tumor Models

2×10⁵ KPC1 or KP/cells were resuspended in 25 μl of Matrigel (Matrigel, BD) diluted 1:1 with cold PBS and transplanted directly into the liver of 8-10 week old C57BL/6 female mice. Following tumor development in the liver (6-8 days post-transplantation), mice were evaluated by BLI on a Xenogen IVIS (Caliper Life Sciences) to quantify liver tumor burden before being randomized into various study cohorts. Upon sacrifice liver tumor tissue was allocated for 10% formalin fixation, OCT frozen blocks, and flow cytometry analysis.

### KPC GEMM Model

Trp53^{fl/+}, Kras^{+/LSL-G12D} and P48-Cre strains on a C57B1/6 background were interbred to obtain P48-Cre; Kras^{+/LSL-G12D}; Trp53^{fl/+} (KPC) GEMM mice. Mice were monitored for tumor development by ultrasound imaging, and enrolled and randomized into treatment groups once tumors reached ˜50 mm³ in volume. Upon sacrifice pancreas tumor tissue was allocated for 10% formalin fixation and OCT frozen blocks.

### Preclinical Drug Studies

Mice were treated with vehicle, trametinib (1 mg/kg body weight), palbociclib (100 mg/kg body weight) and/or tazemetostat (125 mg/kg (low) or 400 mg/kg (high) body weight) per os for 4 consecutive days followed by 3 days off treatment. For NK and T cell depletion, mice were injected intraperitoneally (IP) with an αNK1.1 (250 μg; PK136, BioXcell), αCD8 (200 μg; 2.43, BioXcell) or αCD4 (200 μg; GK1.5, BioXcell) antibody twice per week. Depletion of NK, CD4⁺, and CD8⁺ T cells was confirmed by flow cytometric analysis. For neutralization of chemokine signaling, mice were injected IP with an αCCL2 (200 μg; 2H5, BioXcell) or αCXCR3 (200 μg; CXCR3-173, BioXcell) antibody twice per week. For depletion of SMA⁺ fibroblast in SMA-TK mice, mice were injected IP with GCV (50 mg/kg, InvivoGen) daily. No obvious toxicities were observed in treated animals. Ultrasound imaging was repeated every 2 weeks during treatment to assess changes in PDAC tumor burden.

### Ultrasound Imaging

High-contrast ultrasound imaging was performed on a Vevo 3100 System with a MS250 13- to 24-MHz scanhead (VisualSonics) to stage and quantify PDAC tumor burden. Tumor volume was analyzed using Vevo 3100 software.

### Bioluminescence Imaging

Bioluminescence imaging (BLI) was used to track luciferase expression in transplanted KPC1 PDAC and KP1 LUAD tumor cells expressing a luciferase-GFP reporter. Mice were injected IP with luciferin (5 mg/mouse; Gold Technologies) and then imaged on a Xenogen IVIS Spectrum imager (PerkinElmer) 10-15 minutes later for 60 seconds. Quantification of luciferase signaling was analyzed using Living Image software (Caliper Life Sciences).

### Flow Cytometry

For analysis of MHC-I expression in cell lines cultured in vitro, cells were treated for 8 days with vehicle (DMSO), combined trametinib (25 nM) and palbociclib (500 nM), and/or tazmetostat (5 μM) and then trypsinized, resuspended in PBS supplemented with 2% FBS, and stained with an H-2k^{b} antibody (AF6-88.5.5.3, eBioscience) for 30 minutes on ice. Flow cytometry was performed on a BD LSR II, and data were analyzed using FlowJo (TreeStar).

For in vivo sample preparation, lungs were isolated, flushed with PBS, and allocated for 10% formalin fixation (1 lobe), OCT frozen blocks (1 lobe), and FACS (3 lobes) following 2-week treatment. Pancreatic tumor tissue was isolated from the spleen and normal tissue and allocated for 10% formalin fixation, OCT frozen blocks, and FACS following 2-week treatment. Liver tumors were isolated from liver lobes and allocated for 10% formalin fixation, OCT frozen blocks, and FACS following 2-week treatment. To prepare single cell suspensions for flow cytometry analysis, lung, pancreas, or liver tissue was minced with scissors into small pieces and placed in 5 ml of collagenase buffer (Ix HBSS w/calcium and magnesium (Gibco), 1 mg/ml Collagenase A (Roche) for LUAD tumors or Collagenase V (Sigma-Aldrich) for PDAC tumors, and 0.1 mg/ml DNase I) in C tubes and then processed using program 37C_m_LDK_1 (for LUAD tumors) or 37C_m_TDK 1_1 (for PDAC tumors) on a gentleMACS Octo dissociator with heaters (Miltenyi Biotec). Spleens were placed in 3 ml of PBS supplemented with 2% FBS in C tubes and dissociated using program m_spleen_01 on a gentleMACS Octo dissociator with heaters (Miltenyi Biotec). Dissociated tissue was passaged through a 70 μm cell strainer and centrifuged at 1500 rpm×5 minutes. Red blood cells were lysed with ACK lysis buffer (Quality Biological) for 5 minutes, and samples were centrifuged and resuspended in PBS supplemented with 2% FBS. Samples were blocked with anti-CD16/32 (FC block, BD Pharmigen) for 20 minutes and then incubated with the following antibodies for 30 minutes on ice: CD45 (30-F11), NK1.1 (PK136), CD3 (17A2), CD8 (53-6.7), CD4 (GK1.5), CD69 (H1.2F3), Sca-1 (Ly6A/E; D7), F4/80 (BM8) (Biolegend); and CD11b (M170) BD Biosciences). NK cells were gated from the CD45⁺CD3⁻NK1.1⁺ population. DAPI was used to distinguish live/dead cells, and tumor cells were gated as GFP⁺. Flow cytometry was performed on an BD LSRFortessa or LSR II, and data were analyzed using FlowJo (TreeStar).

For analysis of Granzyme B (GZMB) expression in NK and T cells, single cell suspensions from tumor tissue were resuspended in RPMI media supplemented with 10% FBS and 100 IU/ml P/S and incubated for 4 hours with PMA (20 ng/ml, Sigma-Aldrich), Ionomycin (1 μg/ml, STEMCELL technologies), and monensin (2 μM, Biolegend) in a humidified incubator at 37° C. with 5% CO₂. Cell surface staining was first performed with CD45 (30-F11), NK1.1 (PK136), CD3 (17A2), CD8 (53-6.7), and CD4 (GK1.5) antibodies (Biolegend). Intracellular staining was then performed using the Foxp3/transcription factor staining buffer set (eBioscience), where cells were fixed, permeabilized, and then stained with a GZMB antibody (GB11; Biolegend). GZMB expression was evaluated by gating on CD3⁻NK1.1⁺ NK cells and CD3⁺CD8⁺ T cells on an BD LSR II flow cytometer as described above.

### NK and T Cell Degranulation Assays

Mice were injected intravenously (i.v.) with 250 μl of a solution containing 25 μg anti-CD107a PE (ID4B, Biolegend) and 10 μg monensin (Biolegend) in PBS 4 hours before mice were euthanized. Tumor tissue was then isolated, dissociated into single cell suspensions, stained with cell surface antibodies, and analyzed by flow cytometry as described above.

### Immunohistochemistry (IHC)

Tissues were fixed overnight in 10% formalin, embedded in paraffin, and cut into 5 μm sections. Haematoxylin and eosin (H&E), Masson's trichrome, and immunohistochemical staining were performed using standard protocols. Sections were de-paraffinized, rehydrated, and boiled in a pressure cooker for 20 minutes in 10 mM citrate buffer (pH 6.0) for antigen retrieval. Antibodies were incubated overnight at 4° C. The following primary antibodies were used: pERK^{T202/Y204} (4370), EZH2 (5246) (Cell Signaling); Ki67 (AB16667), H3K27me3 (AB177178), CD3 (AB5690), GZMB (AB4059), CD31 (AB28364) (Abcam); pRB^{S807/S811} (Sc-16670, Santa Cruz); and NKp46 (AF2225, R&D Systems). HRP-conjugated secondary antibodies (Vectastain Elite ABC Kits: PK-6200; Rabbit, PK-6101; Goat, PK-6105) were applied for 30 minutes and visualized with DAB (Vector Laboratories; SK-4100). For quantification of CD31⁺ blood vessels and NKp46⁺, CD3⁺, and GZMB⁺ immune cells, 5-10 high power 20× fields per section were counted and averaged.

### High Throughput RNA-Sequencing (RNA-seq)

For RNA-seq analysis of PIP, PIL, LIL, and LIP tumor samples, GFP⁺ tumor cells were FACS sorted on a FACSAria (BD Biosciences) from the lungs or pancreas of tumor-bearing mice following 2-week treatment with vehicle or combined trametinib (1 mg/kg body weight) and palbociclib (100 mg/kg). Total RNA was extracted from tumor cells using the RNeasy Mini Kit (Qiagen). Purified polyA mRNA was subsequently fragmented, and first and second strand cDNA synthesis performed using standard Illumina mRNA TruSeq library preparation protocols. Double stranded cDNA was subsequently processed for TruSeq dual-index Illumina library generation. For sequencing, pooled multiplexed libraries were run on a HiSeq 2500 machine on RAPID mode. Approximately 10 million 76 bp single-end reads were retrieved per replicate condition. RNA-Seq data was analyzed by removing adaptor sequences using Trimmomatic⁸⁵, aligning sequencing data to GRCh37.75(hg19) with STAR⁸⁶, and genome wide transcript counting using HTSeq⁸⁷ to generate a RPKM matrix of transcript counts. Genes were identified as differentially expressed using R package DESeq2 with a cutoff so of absolute log₂FoldChange≥1 and adjusted p-value <0.05 between experimental conditions⁷⁸. For heatmap visualization of selected genes and pathways, samples were z-score normalized and plotted using ‘pheatmap’ package in R. Heatmaps display the fold change in RPKM expression values from T/P-treated samples normalized to vehicle-treated samples (T/P-V). Over-representation analysis of DEGs against KEGG⁸⁸ Pathways was performed using clusterProfiler⁸³.

For RNA-seq analysis of shRen and shEzh2 PDAC tumor samples or those produced in SMA-TK mice, GFP⁺ tumor cells were FACS sorted on a FACSAria (BD Biosciences) from the pancreas of tumor-bearing mice following 2-week treatment with vehicle or combined trametinib (1 mg/kg body weight) and palbociclib (100 mg/kg). Total RNA was extracted from tumor cells using the RNeasy Mini Kit (Qiagen). Library preparation and sequencing on a NovaSeq 6000 was performed by Novogene. Approximately 30 million 150 bp paired-end reads were retrieved per replicate condition. Quality of raw sequencing data was checked using FastQC (bioinformatics.babraham.ac.uk/projects/fastqc/) to assure data quality. Paired-end reads were aligned to the mouse reference genome GRCm38 (Ensembl, version 101) using STAR⁸⁶. A gene-by-sample count matrix was generated using featureCounts⁸⁹. All downstream statistical analyses were done using the R programming language⁹⁰. Briefly, entries of genes with extremely low expression were first removed from the gene-by-sample count matrix. Differential gene analysis was performed using DESeq2⁷⁸ in consideration of surrogate variables for hidden variations, which were identified using svaseq⁷⁹. Genes with absolute values of log₂ (fold change) greater than one and p-values less than 0.05, which were corrected for multiple testing using the Benjamini-Hochberg procedure⁸⁰, were considered as significantly differentially expressed genes (DEGs). Over-representation analysis of DEGs against KEGG Pathways⁸⁸ was performed using clusterProfiler⁸³. Heatmaps were generated using pheatmap (github.com/raivokolde/pheatmap).

To assess expression of SASP genes in human PDAC and LUAD cell lines treated with vehicle (DMSO), trametinib (25 nM), and/or palbociclib (500 nM) for 8 days in culture, we interrogated a previously published RNA-seq dataset under the GEO accession number GSE110397¹⁹. Graphs display normalized RPKM expression values.

### Gene Set Enrichment Analysis (GSEA)

GSEA was performed using the GSEAPreranked tool for conducting gene set enrichment analysis of data derived from RNA-seq experiments (version 2.07) against Hallmark signatures in the MSigDB database (software.broadinstitute.org/gsea/msigdb) and published senescence signatures⁴⁰. The metric scores were calculated using the sign of the fold change multiplied by the inverse of the p-value.

### Transcription Factor Enrichment Analysis

Transcription factor enrichment analysis was performed using gene set libraries from Enrichr⁹¹. Significance of the tests was assessed using combined score, described as c=log(p)*z, where c is the combined score, p is Fisher exact test p-value, and z is z-score for deviation from expected rank.

### Pearson's Correlation Analysis

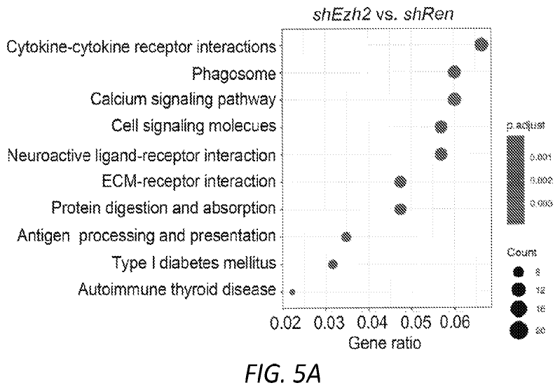

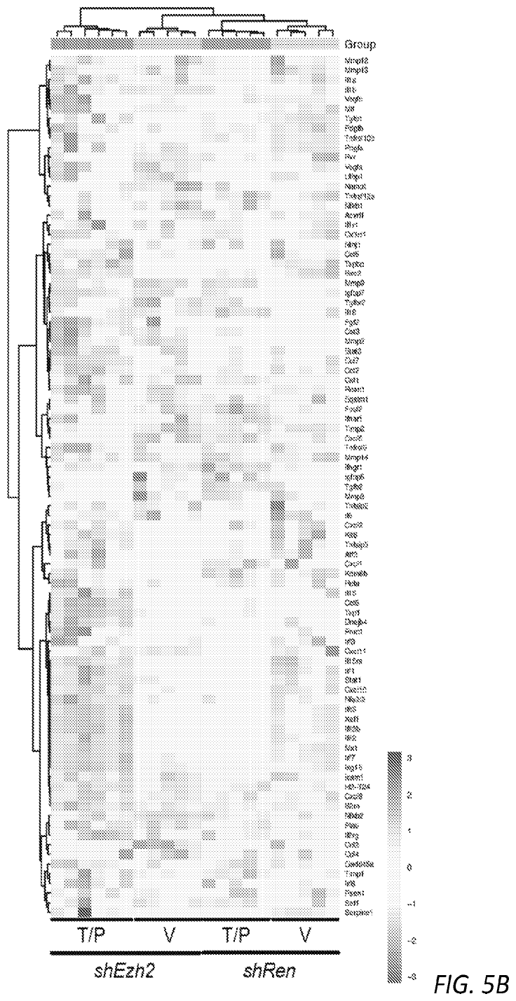

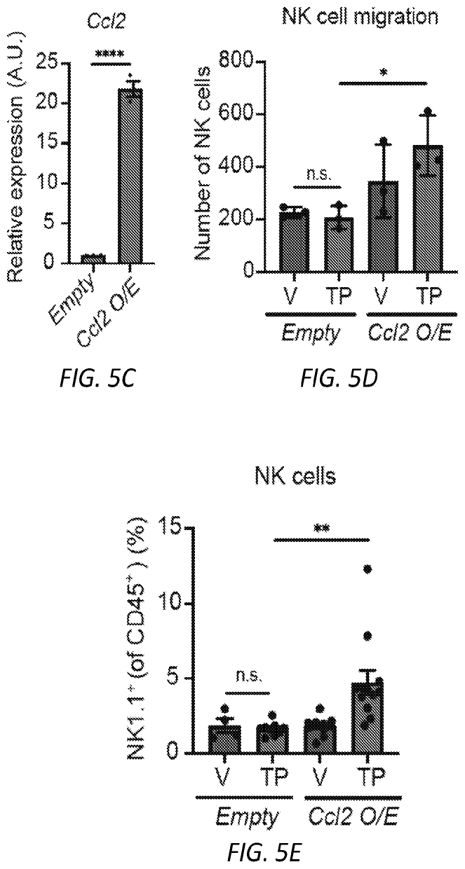

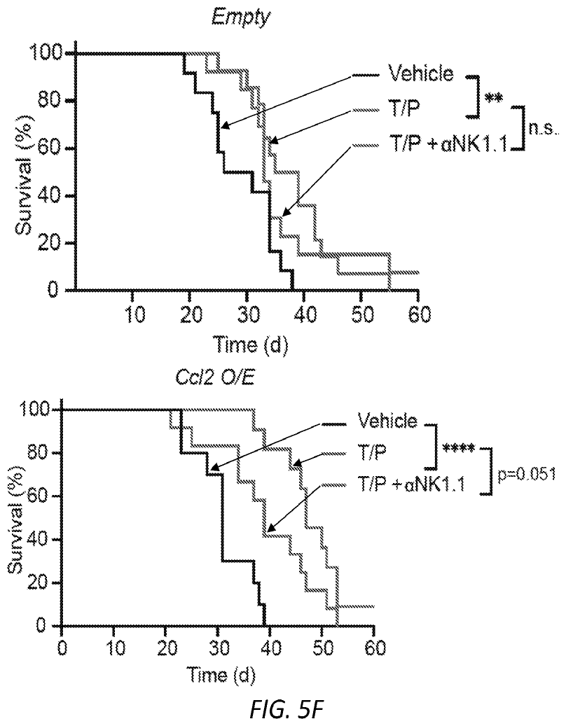

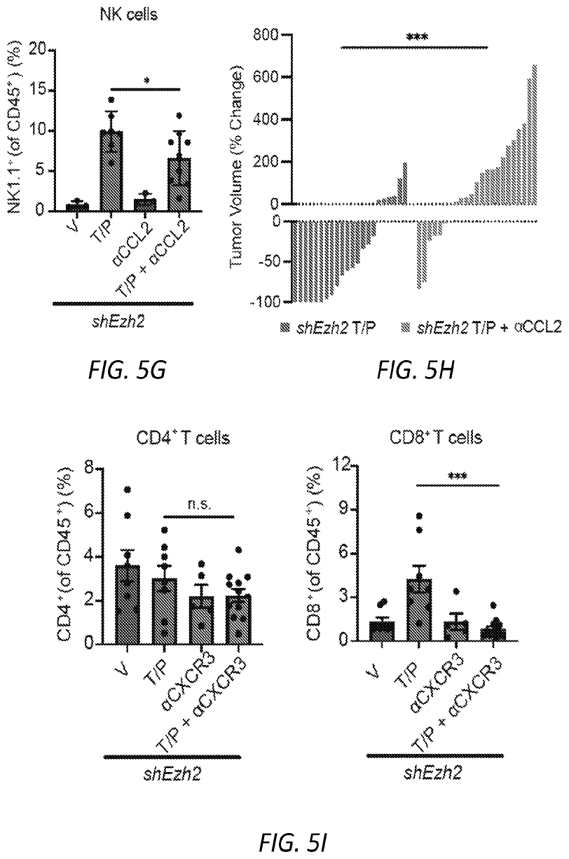

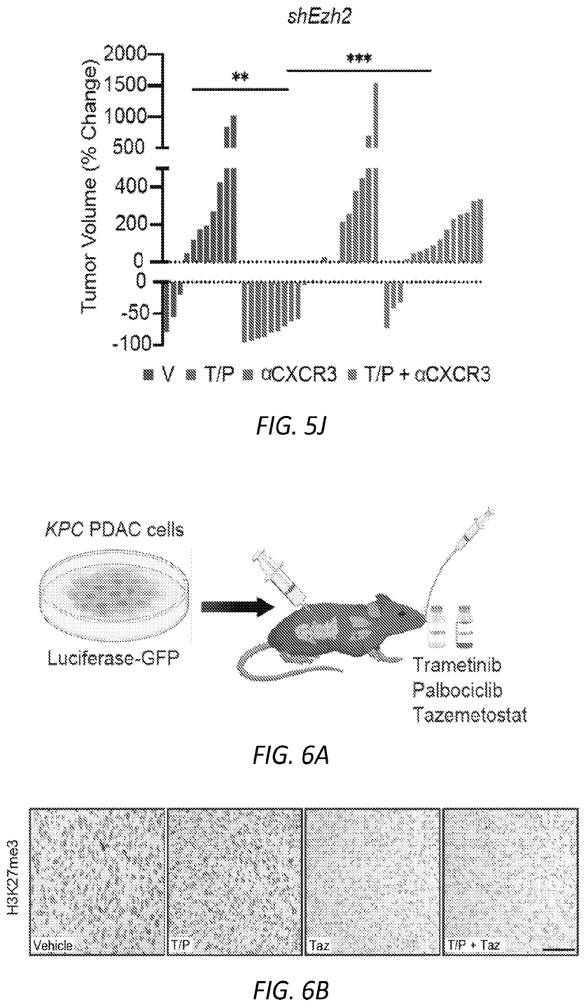

Gene expression data from 145 primary PDAC tumors (GSE71729)⁵⁸ was downloaded with GEOquery2 package. Correlation analysis between PRC2⁹² and our custom EZH2 repression signatures (generated from list of genes downregulated in shEzh2 compared to shRen PDAC tumor cells from RNA-seq analysis in FIG. 5), inflammatory response, NK cell⁹³, and CD8⁺ T cell⁹⁴ gene sets, and CCL2, CXCL9, and CXCL10 expression was performed using ggpubr package. Pearson's correlation coefficient (R) values are displayed.

### Human PDAC Specimens

PDAC patient samples were derived from surgical candidates undergoing Whipple procedures consented under the IRB approved protocol no. H-4721. De-identified FFPE tumor specimens were cut into 5 μm sections and IHC performed as described above to stain for human EZH2 (5246, Cell Signaling) and NKp46 (AF1850, R&D Systems). EZH2 staining was scored as high (strong nuclear staining throughout tumor), intermediate (nuclear staining in some but not all tumor areas), or low (little to no positive staining in the tumor). NKp46⁺ NK cell numbers were scored as high (>5 cells per 40× field), intermediate (2-4 cells per 40× field), or low (<2 cells per 40× field). Survival data from some PDAC patients was also available through the IRB approved protocol no. H-4721.

### Statistical Analysis

Statistical analyses were performed as described in the figure legend for each experiment. Data are expressed as mean±s.e.m. Group size was determined on the basis of the results of preliminary experiments and no statistical method was used to predetermine sample size. The indicated sample size (n) represents biological replicates. Group allocation and outcome assessment were not performed in a blinded manner. All samples that met proper experimental conditions were included in the analysis. All experiments were repeated independently 2-3 times. Survival was measured using the Kaplan-Meier method. Statistical significance was determined by Student's t-test and log-rank test using Prism 6 software (GraphPad Software) as indicated. Significance was set at P<0.05.

### Data Availability

RNA-seq and CUT&Tag data have been deposited in the Gene Expression Omnibus (GEO) under accession nos. GSE201495, GSE141684, and GSE203623. Gene expression data for human LUAD and PDAC cell lines treated with T/P were obtained under accession no. GSE110397. Gene expression data from 145 primary human PDAC specimens were obtained under accession no. GSE71729.

### Example 1. NK Cell Immunity is Induced in the Lung but not Pancreas TME Following Therapy-Induced Senescence

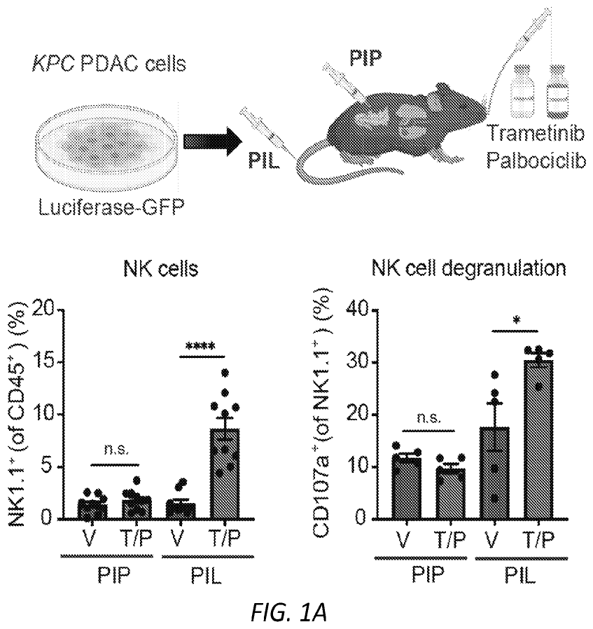

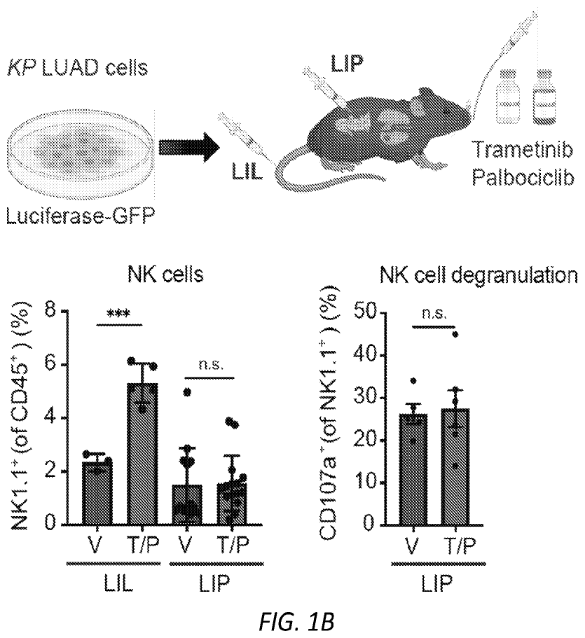

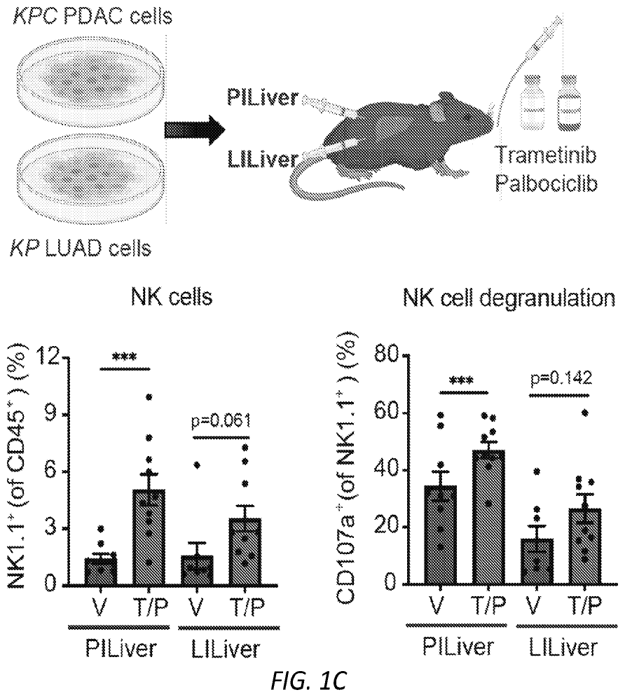

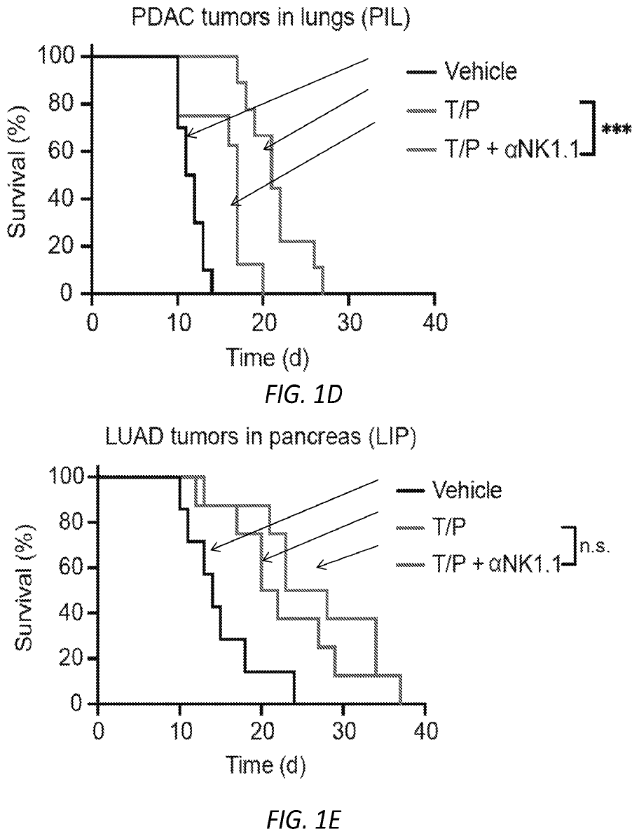

Based on our previous findings we hypothesized that the pancreas TME may contribute to suppression of NK cell anti-tumor immunity following therapy-induced senescence. To test this, we took advantage of genetically similar KRAS mutant PDAC and LUAD cell lines that could be transplanted syngeneically into different organs of C57BL/6 mice to study the impact of the TME on senescence-driven immune responses. These included: (a) KPC PDAC tumor cell lines (KPC1, KPC2) derived from PDAC tumors in Pdx1-Cre; LSL-KRAS^{G12D}; Trp53^{R172H/wt} genetically engineered mouse models (GEMMs)³¹ and (b) KP LUAD cell lines (KP1, KP2) derived from lung tumors in Kras^{LSL-G12D/wt}; Trp53^{flox/flox} GEMM mice administered an adenovirus expressing Cre-recombinase intratracheally³². KPC PDAC or KP LUAD cells were engineered to express luciferase and GFP (to track tumors in vivo) and then transplanted intravenously (i.v.) to form tumors in the lungs or injected directly into the pancreas of C57BL/6 mice (FIG. 1a,b). Additionally, PDAC and LUAD cells were also transplanted into the liver, a common site of metastasis for both tumor types (FIG. 1c). Following tumor formation, mice were treated with vehicle or T/P for two weeks to induce senescence (FIG. 1a-c). PDAC and LUAD tumors propagated in each organ had a similar tumor burden and disease histopathology, as well as senescence (senescence-associated β-galactosidase (SA-β-gal)), anti-proliferative (Ki67), and on-target drug responses (pERK, pRb) to T/P treatment.

In line with our previous findings, T/P treatment led to increased NK cell accumulation and cytotoxicity, as marked by the degranulation markers CD107a and Granzyme B (GZMB), in LUAD tumors grown in the lungs (LIL) but not in PDAC tumors grown in the pancreas (PIP) despite peripheral NK cell expansion in adjacent spleens (FIG. 1a,b). NK cell suppression was specific to the pancreas TME, as PDAC tumors grown in the lungs (PIL) or liver (PILiver) underwent NK cell surveillance following T/P-induced senescence (FIG. 1a,c). Similarly, whereas LUAD tumors propagated in the liver (LILiver) were infiltrated with activated NK cells, those propagated in the pancreas (LIP) were not (FIG. 1b,c). These tissue-specific changes in NK cell states were functionally relevant, as NK cell depletion with an NK1.1-targeting antibody (PK136) reduced the survival benefit of T/P treatment in mice bearing tumors in the lungs (PIL) but not those with tumors in the pancreas (LIP, PIP) (FIG. 1d,e). In contrast, T/P-induced senescence led to increased CD4⁺ and CD8⁺ T cells in all tumor-bearing tissues, though the infiltrating T cells were not activated and T cell depletion studies indicated they did not contribute to anti-tumor immunity in the lung or pancreas TME. Therefore, the pancreas TME leads to specific resistance to NK cell immune surveillance following therapy-induced senescence.

### Example 2. The Pancreas TME Suppresses the Pro-Inflammatory SASP

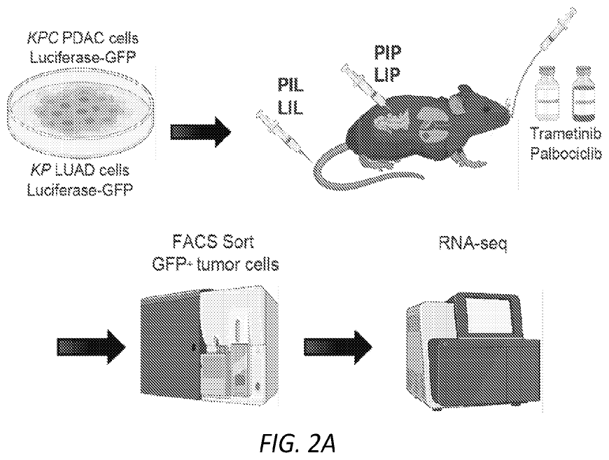

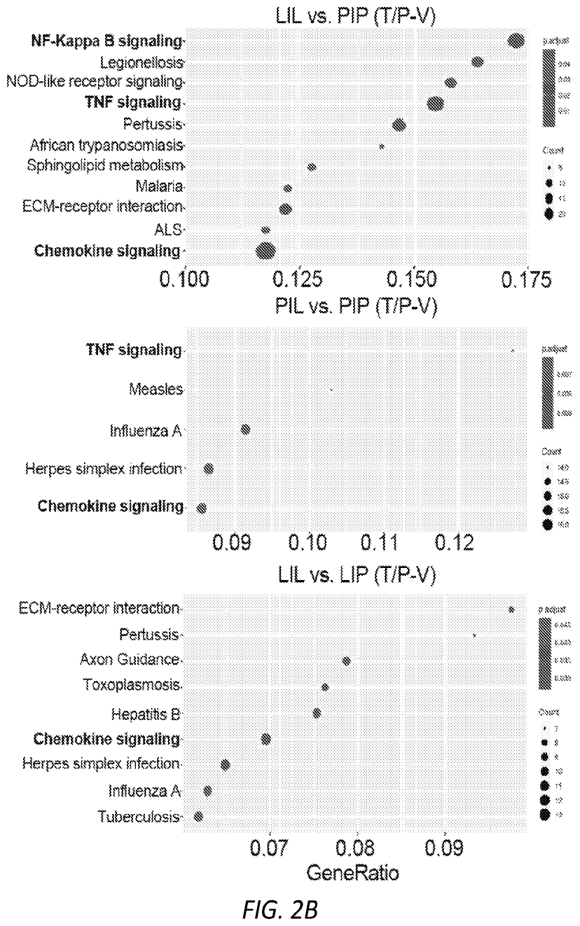

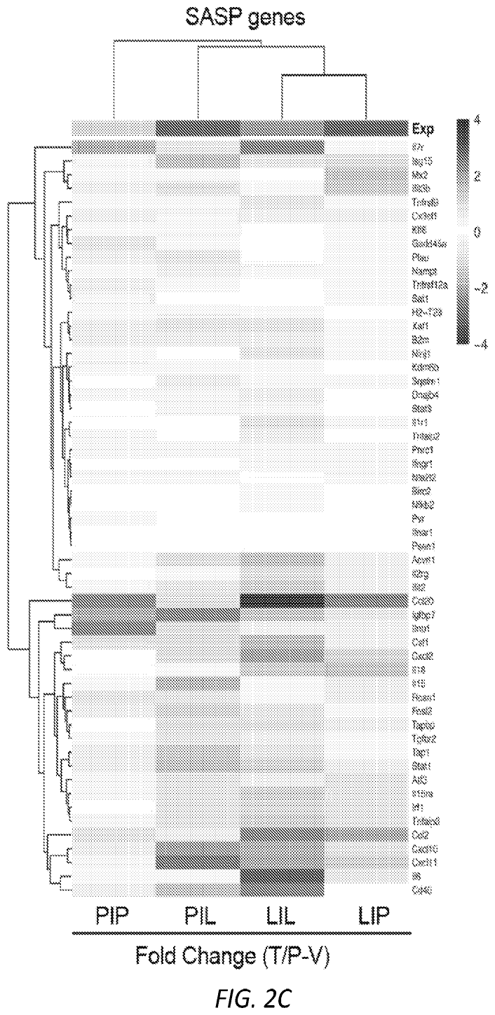

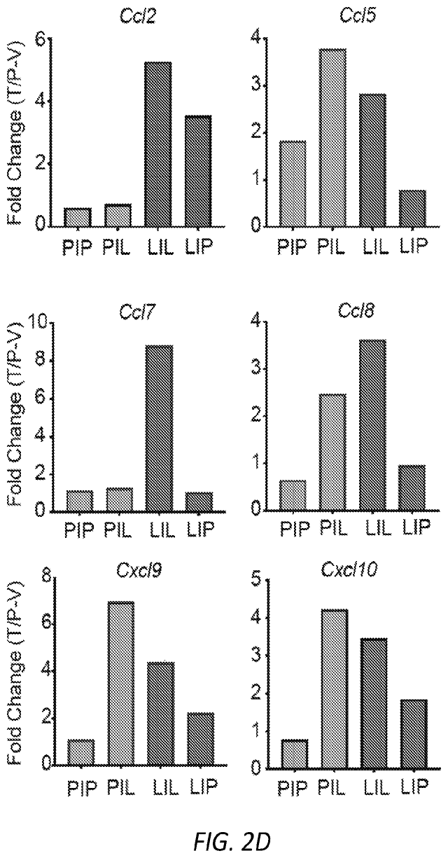

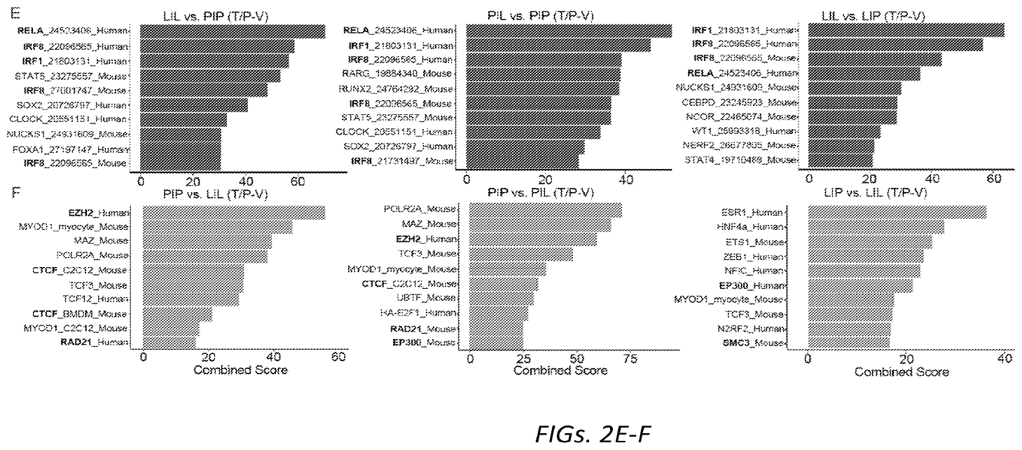

We next performed RNA-sequencing (RNA-seq) on GFP-labeled PDAC and LUAD cells FACS sorted from lung or pancreas tumors following T/P treatment to determine the impact of the TME on signaling in senescent tumor cells (FIG. 2a). T/P treatment led to significant enrichment of inflammatory pathways related to NF-κB, TNF, and chemokine signaling, as well as type I interferon and IL-12 pathways known to activate innate and in particular NK cell immunity, in PDAC and LUAD cells in the lungs (PIL, LIL) as compared to those in the pancreas (LIP, PIP) (FIG. 2b). A subset of pro-inflammatory SASP genes were significantly upregulated following T/P-induced senescence in PDAC and LUAD cells in the lung TME but not those in the pancreas TME (FIG. 2c). These included a number of SASP-related chemokines known to regulate the chemotaxis of NK cells and T cells into tumors, including CCL2, CCL5, CCL7, CCL8, CXCL9, and CXCL10 (FIG. 2d)³³. This pancreas tissue-specific suppression of the SASP output was independent of senescence-associated cell cycle arrest, as T/P treatment uniformly reduced E2F and MYC target gene expression, the Ki67 proliferation index, and other senescence-related markers and gene sets in all tumor conditions tested. In vitro, T/P treatment induced the expression and secretion of pro-inflammatory SASP factors to a similar degree in KPC PDAC and KP LUAD cells and in KRAS mutant human PDAC and LUAD cell lines, suggesting that PDAC cells have the intrinsic capacity to produce pro-inflammatory SASP factors following therapy-induced senescence. Thus, a set of pro-inflammatory SASP genes known to regulate NK cell immune surveillance are transcriptionally repressed in the pancreas TME following therapy-induced senescence.

Senescence induction is associated with dynamic transcriptional and chromatin changes. Rb and p53-regulated pathways mediate repression of cell cycle genes^{34,35}. In addition, a number of other transcription factors and regulators, including NF-κB, C/EBPβ, cGAS-STING, JAK/STATs, and NOTCH, lead to activation of divergent SASP programs³⁶⁻³⁹. These transcriptional changes are accompanied by dramatic remodeling of the chromatin landscape, with Rb enabling repressive H3K9me3-mediated chromatin compaction at cell cycle genes³⁵, and BRD4 facilitating H3K27Ac-mediated enhancer activation at SASP loci⁴⁰. Transcription factor enrichment analysis demonstrated that transcriptional targets of NF-κB and its p65 subunit RELA, which we have shown to be important activators of the SASP following T/P-induced senescence^{19,20}, were induced preferentially in tumors propagated in the lung TME (FIG. 2e). Targets of interferon regulatory factors (IRFs) that respond to and drive interferon production downstream of STING pathway activation were also enriched in tumors in the lung as compared to pancreas TME (FIG. 2e). Interestingly, regulators of 3D chromatin topology and DNA looping (CTCF, RAD21, SMC3), as well as histone modifications and chromatin compaction (EZH2, p300), were enriched in tumors within the pancreas TME (FIG. 2f). These findings suggested the possibility that chromatin remodeling within tumors in the pancreas TME may lead to transcriptional repression of specific SASP genes and regulators.

### Example 3. EZH2 Methyltransferase Activity Leads to Pro-Inflammatory SASP Suppression in PDAC

EZH2 is a member of the polycomb repressor complex 2 (PRC2) with methyltransferase activity that mediates transcriptional gene repression through H3K27 trimethylation (H3K27me3)⁴¹. Suppression of EZH2 can induce cellular senescence and a subsequent SASP in fibroblasts through activation of DNA-damage response (DDR) pathways and loss of repressive H3K27me3 marks on (CDKN2A (i.e. p16), a canonical regulator of senescence-associated cell cycle arrest, and other SASP gene loci⁴²⁻⁴⁴. In addition, EZH2 induction in other cancer settings mediates suppression of NK and T cell immune surveillance and immunotherapy resistance⁴⁵⁻⁴⁸. We found expression of EZH2 repressed genes significantly decreased, and global levels of H3K27me3 dramatically increased, in tumors propagated in the pancreas as compared to those in the lungs. Based on these findings, we hypothesized that EZH2 activation may lead to epigenetic silencing of the SASP through its methyltransferase activity that contributes to suppression of cytotoxic lymphocyte immunity in PDAC.

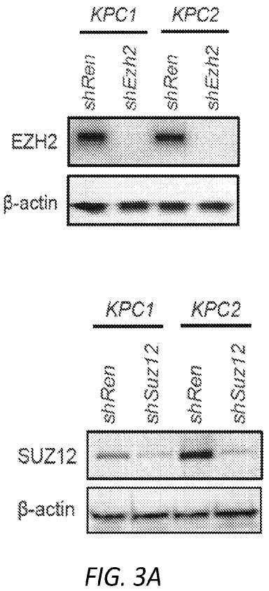

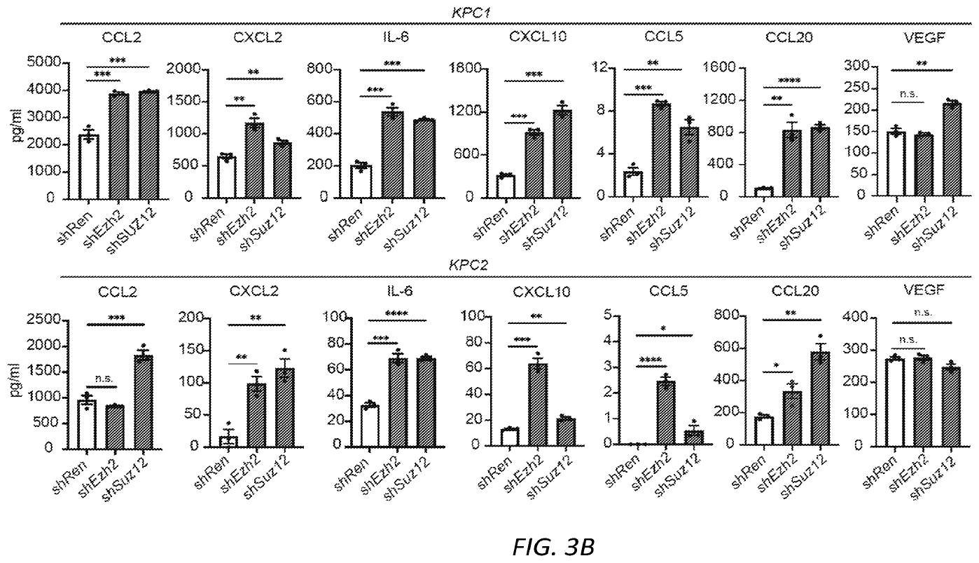

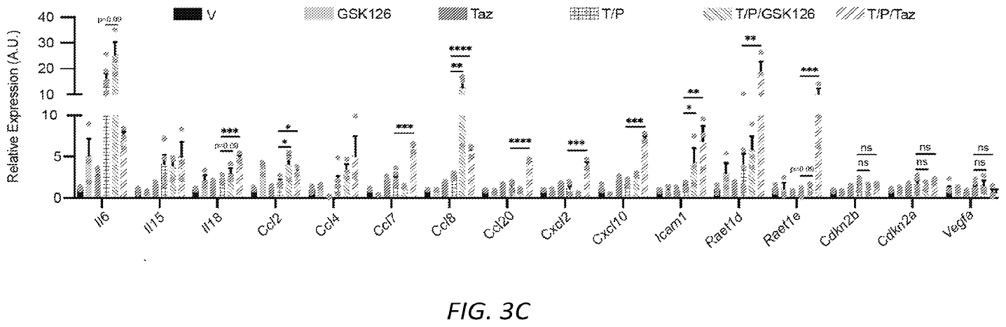

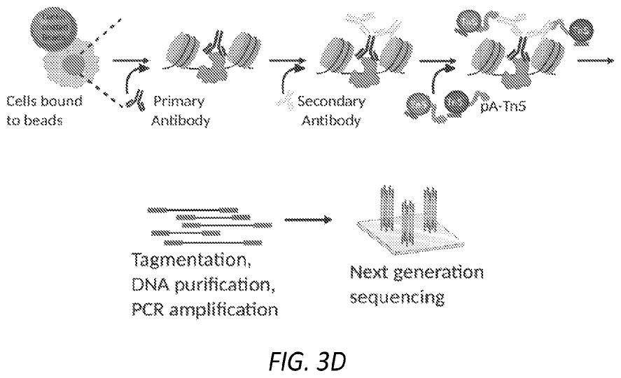

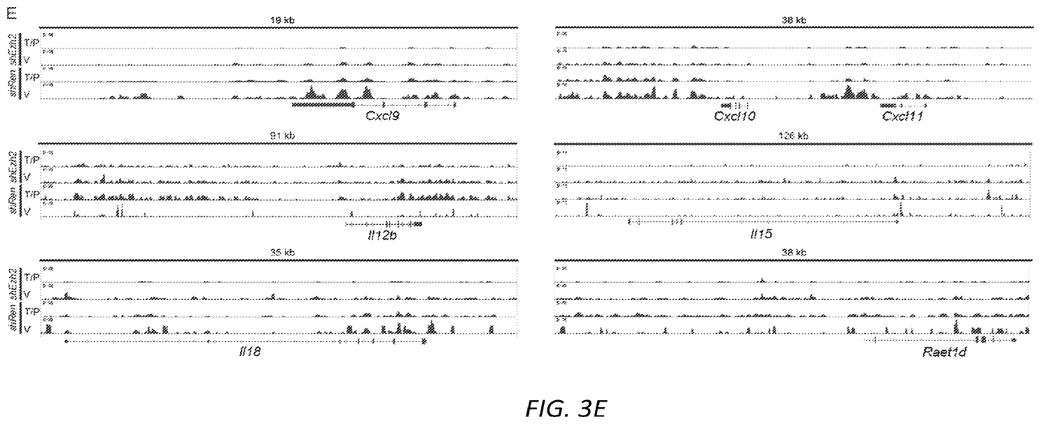

Short hairpin RNAs (shRNAs) were generated that could potently suppress levels of EZH2 or SUZ12, another PRC2 complex component that interacts with EZH2, in our KPC PDAC cell lines (FIG. 3a). EZH2 knockdown had no impact on senescence-induced growth arrest or expression of SA-β-gal or other senescence-related genes (e.g. Cdkn2a, Cdkn2b) following T/P treatment in KPC PDAC cells. In contrast, SASP-related pro-inflammatory cytokines (e.g. IL-6, IL-15, IL-18) and chemokines (CCL2/5/7/8/20, CXCL2/10) important for cytotoxic lymphocyte immunity were significantly upregulated at the gene expression and protein secretion levels in KPC PDAC cells harboring Ezh2 or Suz12-targeting shRNAs as compared to those harboring a control Renilla (Ren) shRNA following T/P treatment (FIG. 3b. Immunomodulatory cell surface proteins associated with the SASP, including cell adhesion molecules (ICAM-1) important for tumor-lymphocyte synapses, stress ligands that bind and stimulate the activating NKG2D receptor on NK cells (RAETs, ULBP1, H60s), and MHC-I necessary for antigen presentation to T cells, were also strongly upregulated following therapy-induced senescence and EZH2 knockdown.

This increased expression of inflammatory molecules observed following EZH2 inhibition was dependent on EZH2 methyltransferase activity, as treatment with the well-characterized EZH2 methyltransferase inhibitors GSK126 and tazemetostat (Taz) induced pro-inflammatory SASP factors and immunomodulatory cell surface proteins without impacting senescence-associated cell cycle arrest to a similar extent as EZH2 genetic knockdown (FIG. 3c). Pharmacological EZH2 methyltransferase inhibition also led to increased expression of pro-inflammatory SASP and cell surface molecules following T/P-induced senescence in the human PDAC cell line PANC-1. Thus, EZH2 blockade leads to enhanced pro-inflammatory SASP production following therapy-induced senescence in PDAC.

To determine whether chromatin compaction governed by histone modifications contributes directly to SASP gene reprogramming following EZH2 targeting, we performed CUT&Tag analysis⁴⁹ to assess the impact of EZH2 suppression on H3K27me3 occupancy at SASP gene loci (FIG. 3d). This analysis uncovered synergistic H3K27me3 loss at the loci of specific pro-inflammatory SASP and NK cell ligand genes, including Cxcl9, Cxcl10. Cxcl11, Il12, Il15, Il18, and Raet1d, following combined EZH2 knockdown and T/P-induced senescence (FIG. 3e). In contrast, whereas H3K27me3 marks at pro-angiogenic SASP genes such as Vegfa, Pdgfa, Pdgfb, and Mmp9 were reduced following T/P treatment in the control shRen setting, H3K27me3 peaks remained unchanged or even increased at these loci following T/P treatment in the shEzh2 setting, suggesting EZH2 suppression preferentially impacted pro-inflammatory SASP genes. Indeed, VEGF production was not stimulated by EZH2 suppression (FIG. 3b-c). Therefore, suppression of EZH2 methyltransferase activity in combination with therapy-induced senescence can synergistically reverse the epigenetic repression and promote the transcriptional activation of specific pro-inflammatory SASP genes in PDAC.

### Example 4. EZH2 Blockade Activates NK and T Cell-Mediated Long-Term Tumor Control Following Therapy-Induced Senescence in PDAC Models

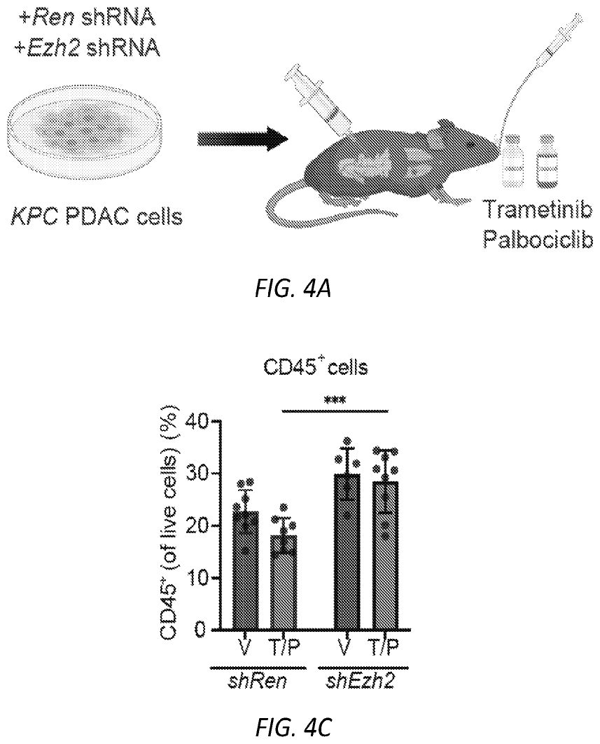

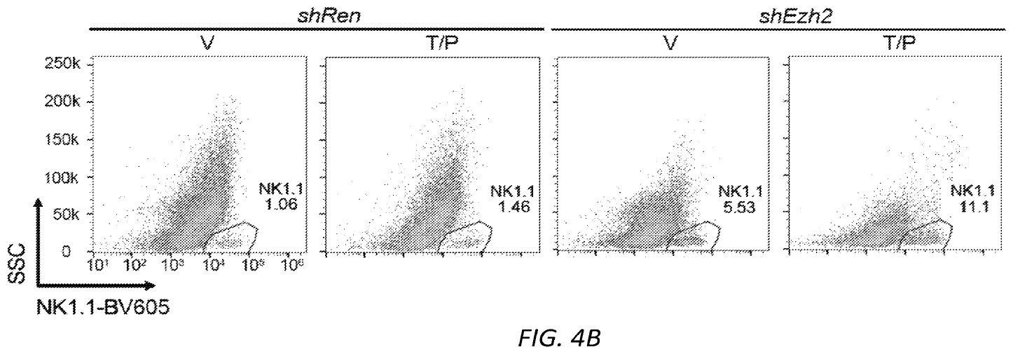

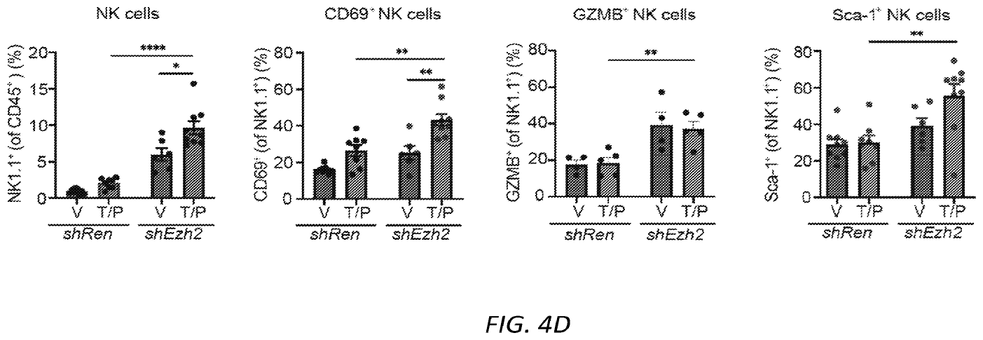

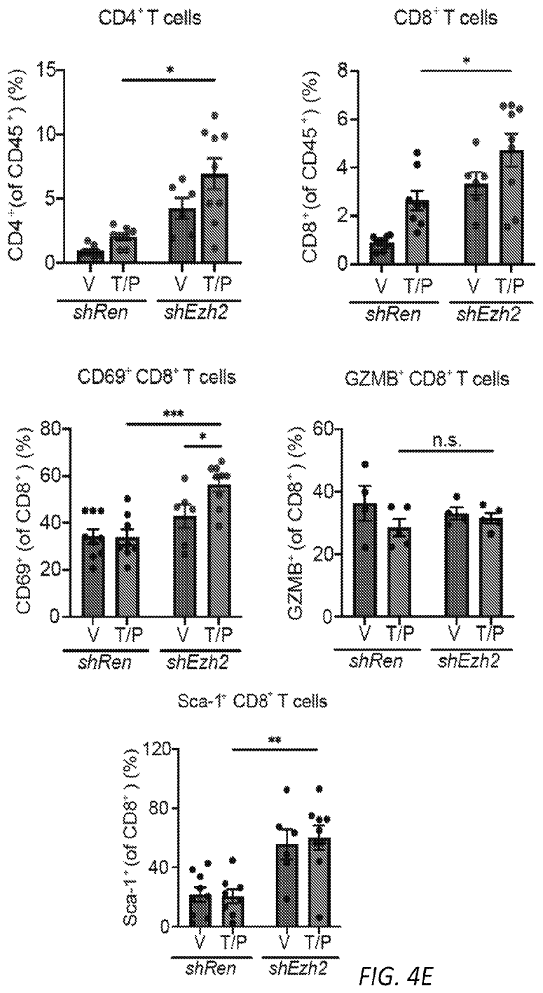

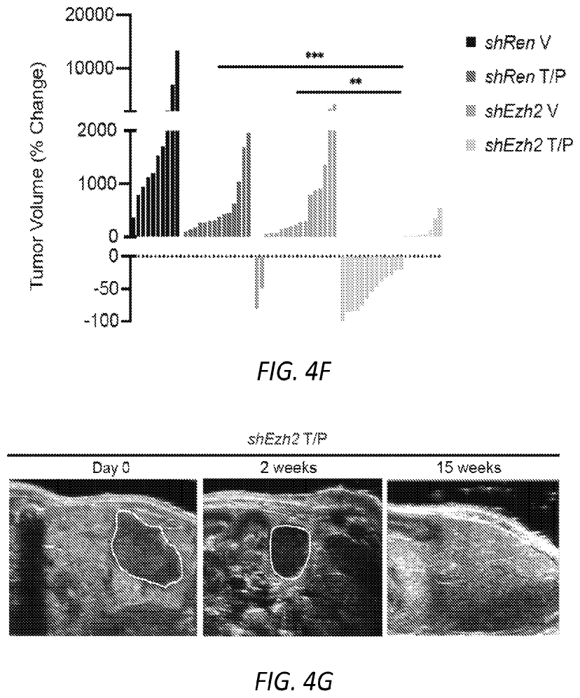

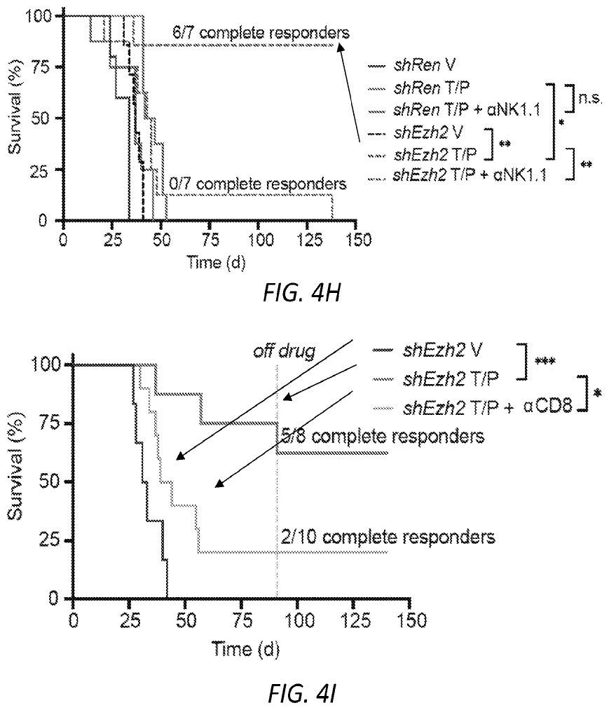

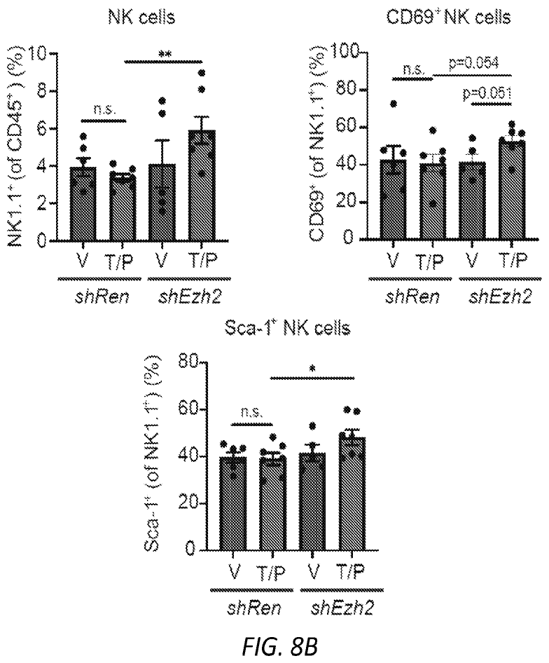

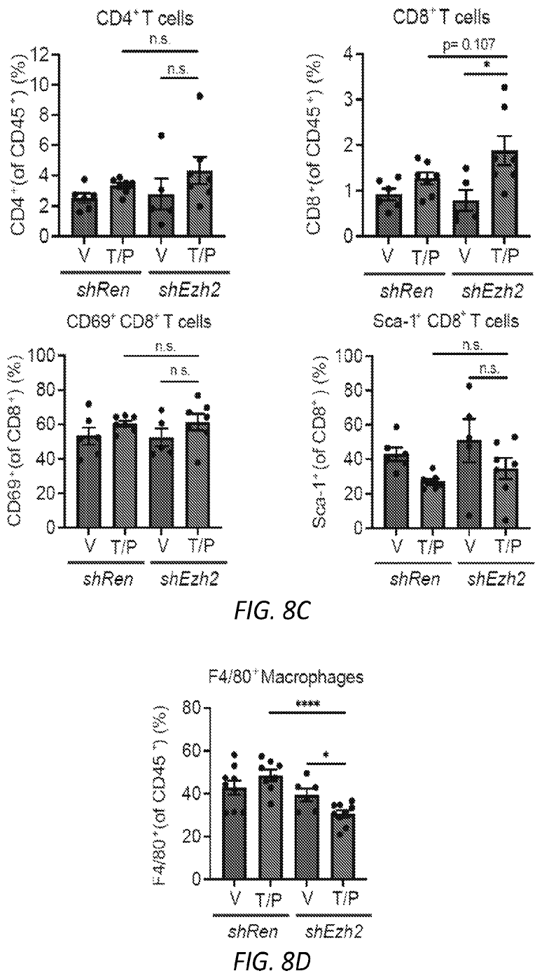

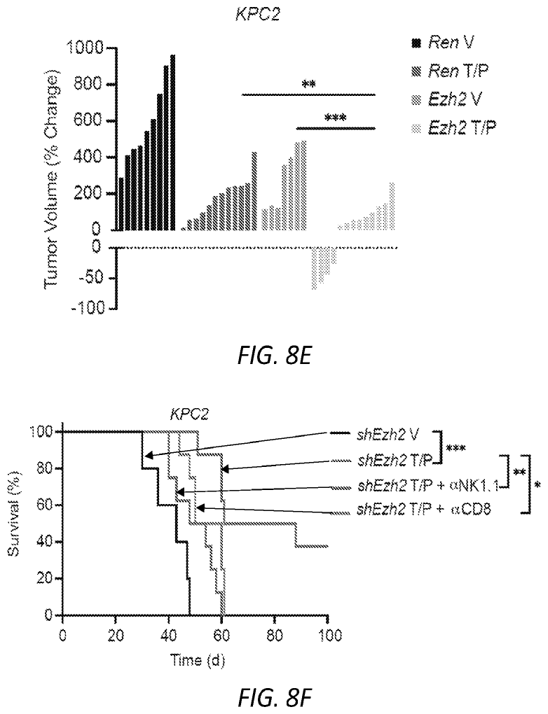

To understand the impact of EZH2 suppression on senescence-mediated anti-tumor immunity in PDAC, we transplanted KPC1 or KPC2 PDAC cells harboring control Renilla (shRen) or EZH2-targeting (shEzh2) shRNAs orthotopically into the pancreas of C57BL/6 mice (FIG. 4a). Transplanted shEzh2 PDAC cells formed tumors at a similar rate as shRen PDAC cells and maintained EZH2 knockdown in vivo (FIG. 8a). Following tumor formation as determined by ultrasound imaging, mice were randomized into treatment groups where they received T/P or a vehicle control (FIG. 4a). Immunophenotyping by multi-parametric flow cytometry analysis following two-week treatment revealed significant changes in lymphocyte numbers and activity. T/P treatment in the setting of EZH2 knockdown led to increased total leukocyte infiltration, including enhanced NK and CD4⁺ and CD8⁺ T cell accumulation (FIG. 4b-e and FIG. 8b,c). NK cells and CD8⁺ T cells also expressed higher levels of early activation (CD69, Sca-1) markers, and NK cells (but not CD8⁺ T cells) appeared more cytotoxic by expression of GZMB following T/P treatment of shEzh2 as compared to control shRen KPC1-derived tumors (FIG. 4d,e and FIG. 8b,c). This increase in cytotoxic lymphocytes following T/P-induced senescence and EZH2 blockade was also accompanied by a decrease in F4/80⁺ macrophages (FIG. 8d).

Combinatorial EZH2 knockdown and T/P treatment also had profound anti-tumor effects. Whereas T/P treatment in the control shRen setting or EZH2 knockdown alone led to a marginal reduction in tumor growth, T/P treatment in the context of EZH2 knockdown produced significant tumor control, with many tumors regressing after just two-week treatment (FIG. 4f,g and FIG. 8e). Remarkably, the majority of shEzh2 KPC1-derived tumors treated with T/P continued to regress and completely responded (FIG. 4g-i). Indeed, while mice harboring control shRen PDAC treated with vehicle or T/P or shEzh2 PDAC treated with vehicle quickly succumbed to the disease, 11/15 mice harboring shEzh2 tumors treated with T/P had complete responses that remained durable even after treatment ceased (FIG. 4h,i). KPC2 PDAC transplant mice also showed enhanced survival following EZH2 knockdown and treatment with T/P, including 3/8 complete responders (FIG. 8f).

We treated some PDAC-bearing mice with NK1.1 (PK136) or CD8 (2.43) depleting monoclonal antibodies (mAbs) simultaneously with drug administration to assess whether activation of NK and/or CD8⁺ T cell immunity was responsible for tumor control. Strikingly, both NK or CD8⁺ T cell depletion mitigated long-term survival and prevented complete tumor responses induced following T/P treatment of animals with EZH2 suppressed KPC1 and KPC2 PDAC tumors (FIG. 4h,i and FIG. 8f). Together these findings demonstrate that EZH2 knockdown can potentiate senescence-mediated long-term tumor control in PDAC through mobilization of cytotoxic NK and T lymphocyte immunity.

### Example 5. EZH2 Suppression Reinstates SASP-Associated Chemokines to Drive NK and T Cell Accumulation in PDAC

Given the numerous cell autonomous and non-cell autonomous functions of EZH2 in cancer biology⁵⁰, we performed bulk RNA-seq on FACS sorted GFP⁺ tumor cells isolated from drug-treated shRen or shEzh2 PDAC tumors to understand the mechanisms by which EZH2 targeting led to immune-mediated tumor control. Unbiased KEGG pathway analysis revealed “cytokine-cytokine receptor interaction” and “cell adhesion molecules” as top differentially regulated pathways when comparing shEZH2 vs. shRen tumors treated with T/P (FIG. 5a). Deeper analysis revealed significantly increased expression of SASP-associated pro-inflammatory cytokines and chemokines (Il15, Ccl2/5/7/8, Cxcl9/10/11, Cx3cl1), as well as genes involved in antigen presentation/processing (B2m, Tap1, Tapbp) and cell adhesion (Icam1) important for T and NK cell recognition of tumor cells in the context of T/P-induced senescence and EZH2 suppression (FIG. 5b).

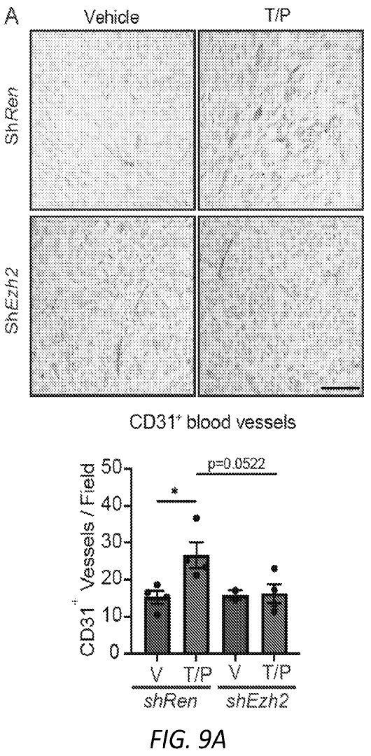

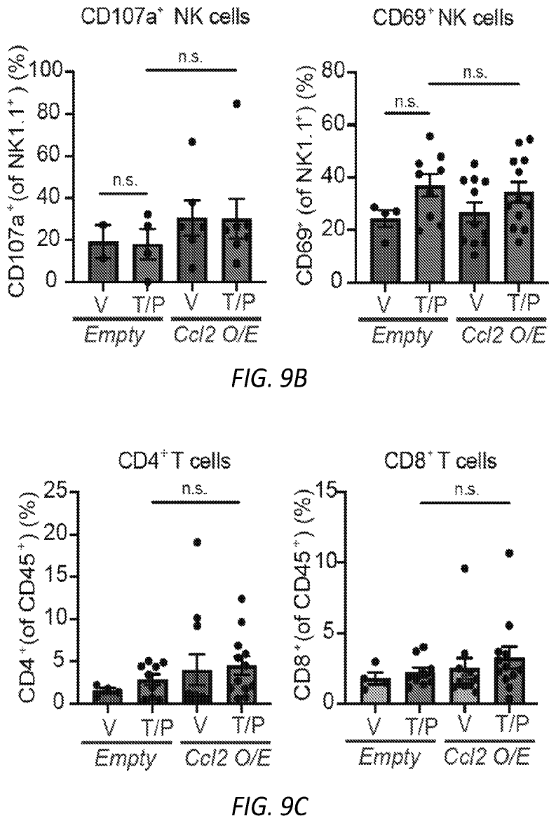

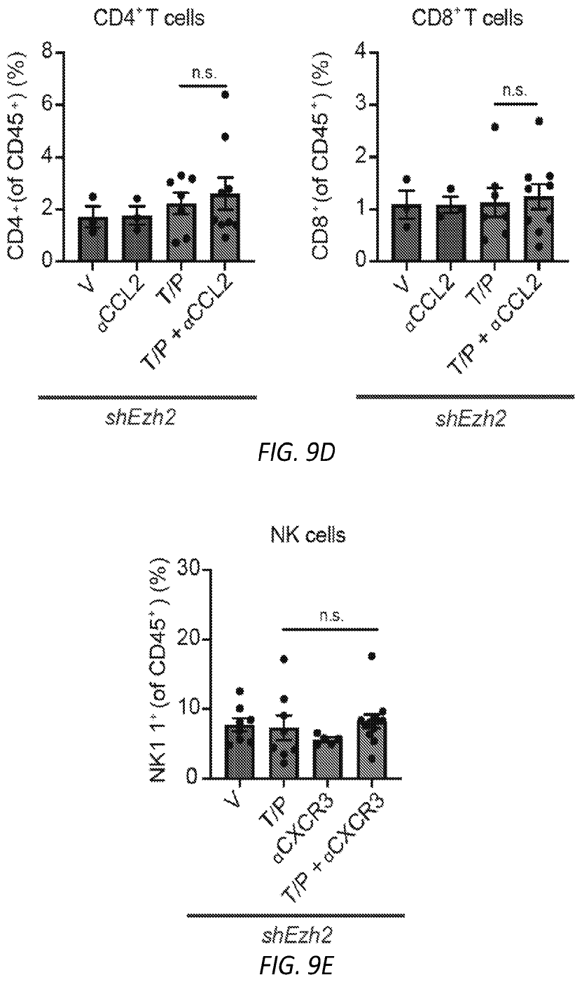

The expression of other SASP-associated factors showed differential responses to EZH2 blockade. Whereas transcriptional regulators of the pro-inflammatory SASP that are normally repressed in the PDAC TME, including STING (Irf1/3/7/8, Ifnar, Isg15) and STAT (Stat1/3) pathway components, were upregulated following therapy-induced senescence and EZH2 suppression (FIG. 5b), many of the pro-angiogenic (Vegfa/b, Pdgfa/b, Mmp3/9/12/13/14) and immune suppressive (Tgfb1/2, Cxcl1/5) SASP factors normally induced during senescence were downregulated (FIG. 5b). Indeed, the increase in blood vessels observed following T/P treatment in shRen tumors and as reported in our previous study²⁰ was not found in shEzh2 tumors (FIG. 9a). Thus, EZH2 suppression following T/P-induced senescence triggers a phenotypic switch in the SASP program in the PDAC TME from a pro-angiogenic SASP to a pro-inflammatory SASP that may contribute to enhanced cytotoxic lymphocyte anti-tumor immunity.

Many of the most highly induced pro-inflammatory SASP factors in the lung TME (FIG. 2c,d) or upon EZH2 knockdown in the PDAC TME (FIG. 5b) following therapy-induced senescence are chemokines known to attract NK and T cells from the periphery into inflamed tissues³³, including CCL2 and CXCL9/10. In addition to its impact on monocyte and macrophage trafficking, CCL2 can also attract NK cells expressing its receptor CCR2⁵¹, as has been previously shown in senescent liver cancer lesions⁵². To interrogate the role of CCL2 in anti-tumor NK cell responses in PDAC, we first engineered KPC PDAC cell lines to express a Ccl2 cDNA (or an Empty vector as a control) (FIG. 5c). In vitro, conditioned media from KPC1 cells overexpressing CCL2 and pre-treated with T/P produced significantly more NK cell migration through a transwell insert (FIG. 5d), demonstrating that CCL2 secretion by senescent tumor cells can facilitate NK cell chemotaxis. Empty or Ccl2 expressing KPC cells were then transplanted orthotopically into the pancreas of C57BL/6 mice and mice randomized into treatment groups following tumor formation to assess the impact on NK cell immune surveillance in vivo. Flow cytometry analysis revealed that tumor-specific CCL2 overexpression in the context of T/P-induced senescence was sufficient to significantly increase NK cell accumulation into PDAC without affecting NK cell activation (FIG. 5e and FIG. 9b). This increased NK cell influx into CCL2 overexpressing tumors prolonged the survival of PDAC-bearing animals treated with T/P, as NK cell depletion with an NK1.1-targeting mAb significantly diminished the survival advantage (FIG. 5f).

To determine whether EZH2 suppression facilitated anti-tumor NK cell immunity through CCL2 induction, we also treated mice transplanted with shEh2 KPC1 PDAC tumors with vehicle, T/P, and/or a mAb targeting CCL2 (2H5). Indeed, CCL2 was required for these effects, as CCL2 blockade resulted in reduced NK cell accumulation and abolished tumor regressions (FIG. 5g,h). Thus, SASP-associated CCL2 is both necessary and sufficient to drive NK cell infiltration and potentiate NK cell-mediated tumor control in PDAC following T/P-induced senescence.

In contrast to its effects on NK cells, CCL2 overexpression or neutralization had little impact on CD4⁺ and CD8⁺ T cell recruitment (FIG. 9c,d), suggesting other SASP chemokines may affect T cell chemotaxis. T cells express the receptor CXCR3 that binds chemokines CXCL9/10/11 that are critical for CD8⁺ T cell homing to the TME and ICB immunotherapy efficacy⁵³⁻⁵⁶. In agreement, treatment of shEzh2 PDAC-bearing mice with vehicle, T/P, and/or a CXCR3 mAb (CXCR3-173) revealed that CXCR3 blockade blunted CD8⁺ and to a lesser extent CD4⁺ T cell accumulation without affecting NK cell numbers (FIG. 5i and FIG. 9e). This reduction in CD8⁺ T cell recruitment upon CXCR3 blockade also mitigated the anti-tumor effects of combined EZH2 suppression and T/P-induced senescence and reversed PDAC tumor regressions (FIG. 5j). Therefore, distinct SASP chemokines are important for NK and T cell chemotaxis and migration into the PDAC TME and enable the immune-mediated anti-tumor effects observed upon EZH2 suppression.

### Example 6. Pharmacological EZH2 Methyltransferase Inhibition in Combination with T/P Reactivates Cytotoxic NK and T Cell Immunity and Enhances Tumor Control in Preclinical PDAC Models

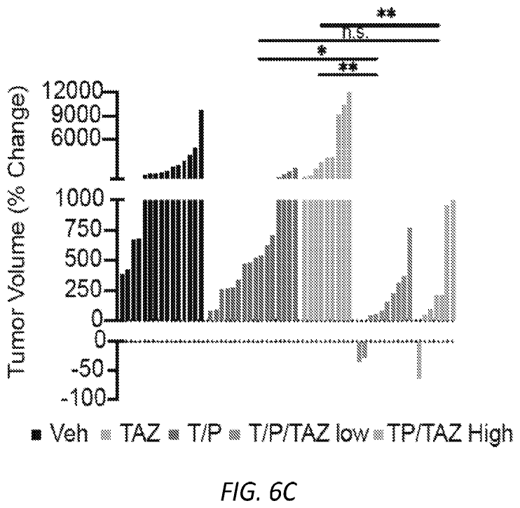

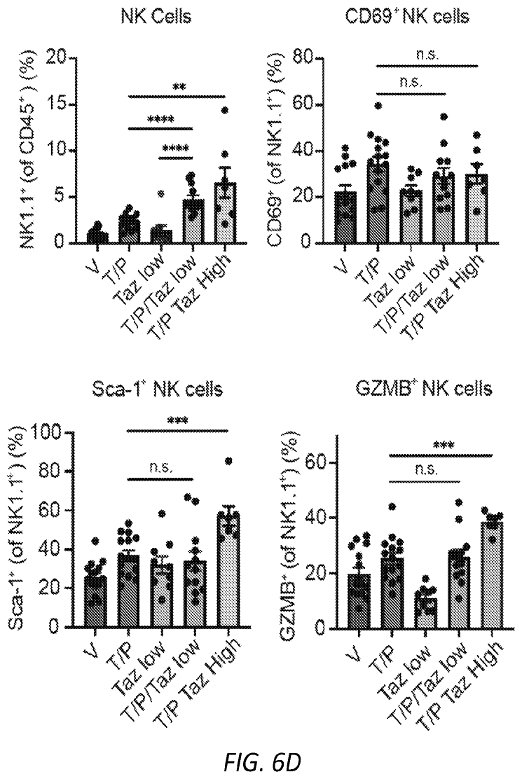

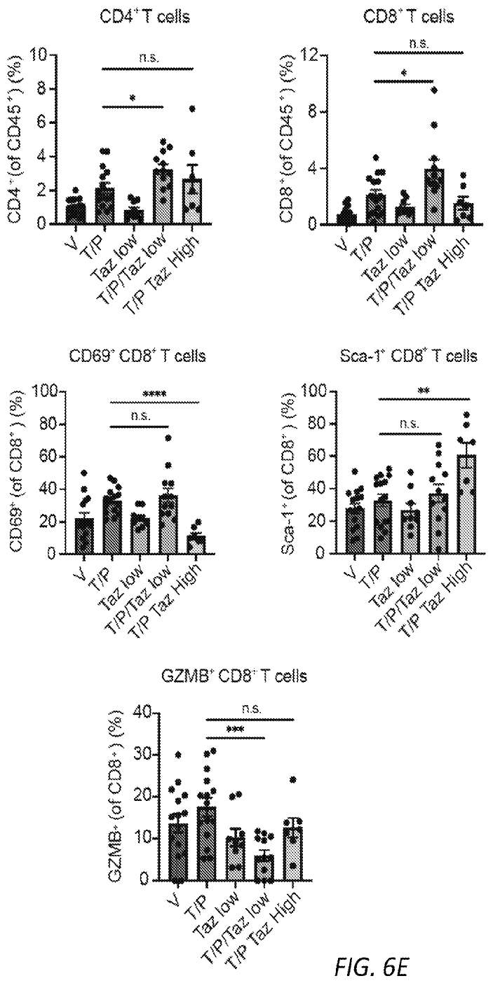

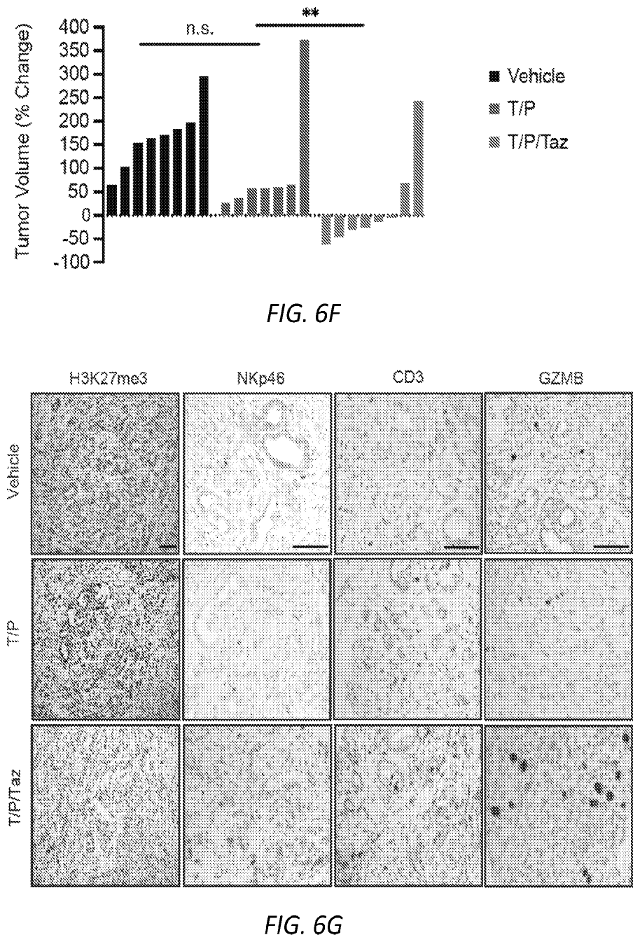

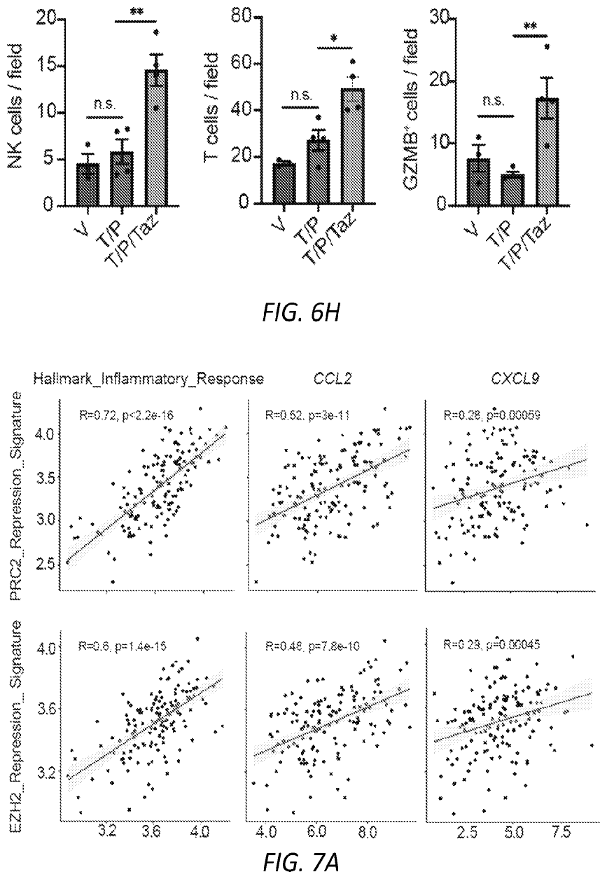

Given the profound anti-tumor effects of genetic EZH2 knockdown in combination with therapy-induced senescence in PDAC, we next tested whether small molecule EZH2 inhibitors that are in clinical development could achieve similar effects. Mice transplanted orthotopically with KPC1 PDAC cells were randomized and treated with vehicle, T/P, and/or the FDA-approved EZH2 methyltransferase inhibitor tazemetostat (Taz) at doses reported to potently inhibit H3K27me3 levels in vivo (FIG. 6a,b). Short-term two-week treatment with Taz in combination with TIP significantly reduced tumor growth compared to each treatment arm alone (FIG. 6c), though tumor regressions were not as frequent or robust as those found with genetic EZH2 knockdown (FIG. 4f). Combined Taz and T/P treatment also led to a significant increase in NK cell numbers and their expression of activation (e.g. Sca-1) and cytotoxicity (e.g. GZMB) markers (FIG. 6d). Interestingly, whereas CD4⁺ and CD8⁺ T cell numbers increased at lower doses of Taz (125 mg/kg) in combination with T/P, high Taz concentrations (400 mg/kg) reduced CD8⁺ T cell numbers and expression of CD69, a marker of early activation and proliferation (FIG. 6d,e). This suggests that, separate from its action on tumor cells, Taz may affect T cell proliferation in a manner that reduces its anti-tumor activity. We therefore performed the remaining studies using the lower 125 mg/kg Taz dose.

To test this inhibitor combination in an autochthonous model, we utilized P48-Cre; LSL-KRAS^{G12D}; Trp53^{fl/wt} (KPC) GEMM mice that spontaneously develop PDAC that closely resembles the human disease⁵⁷. Two-week combined Taz and T/P treatment of tumor-bearing KPC GEMMs led to significantly reduced H3K27me3 levels and tumor regressions in 4/8 mice, in contrast to T/P treatment alone where tumor regressions were not observed (FIG. 6f,g). In addition, Taz treatment in combination with T/P promoted the infiltration of NK cells and enhanced cytotoxic GZMB expression, as well as further increased the T cell accumulation observed with single arm T/P regimens (FIG. 6g,h). Thus, both genetic EZH2 suppression and EZH2 methyltransferase inhibitor treatment can augment T/P-induced senescence to potentiate cytotoxic NK and T cell immunity and tumor control in transplanted and GEMM PDAC models.

### Example 8. EZH2 is Associated with Suppression of Inflammatory Chemokine Signaling, Reduced NK and T Cell Immune Surveillance, and Poor Survival in PDAC Patients

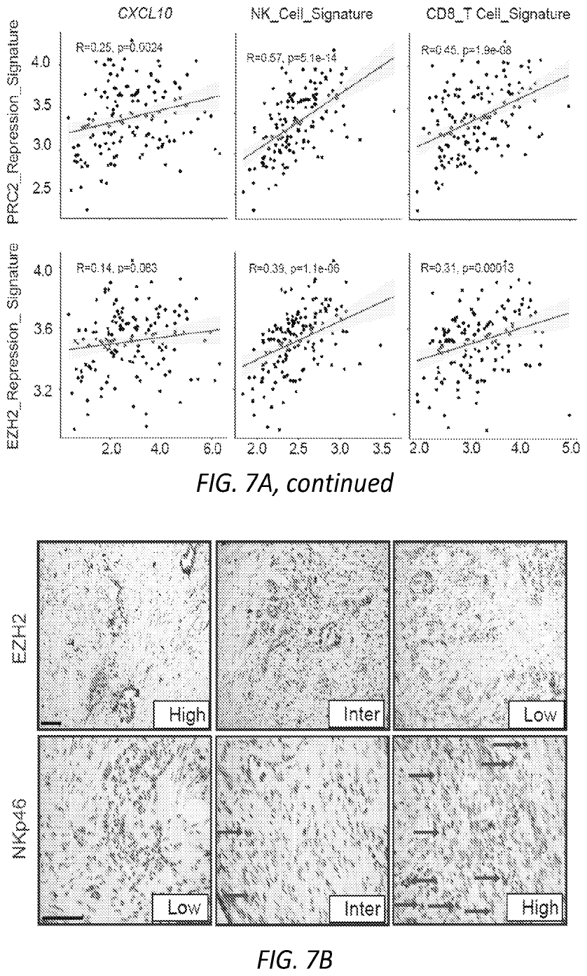

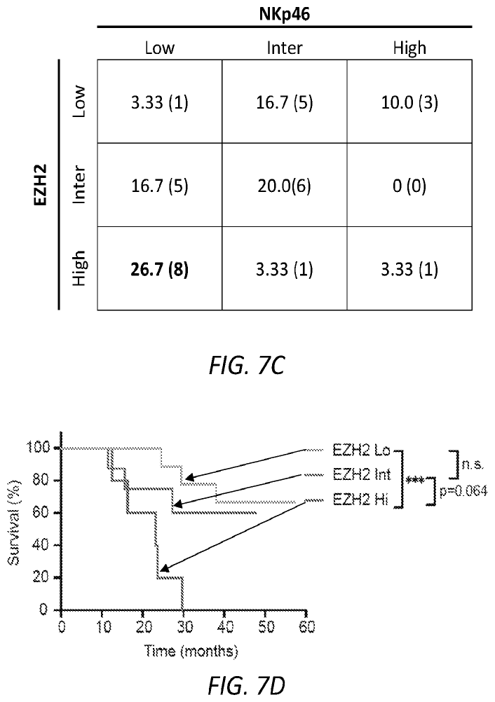

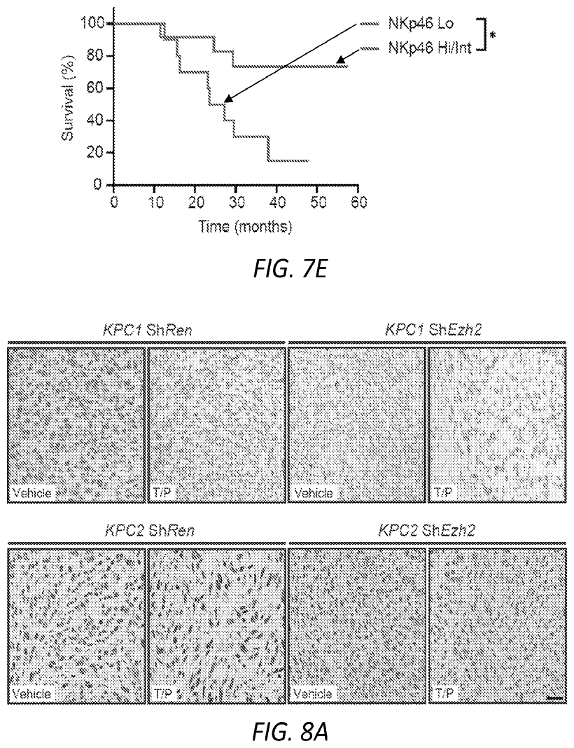

Finally, we set out to evaluate the relationship between EZH2 activity, inflammatory signaling, and NK and T cell immunity in human PDAC. We first interrogated a previously published gene expression dataset containing PDAC patient samples⁵⁸. EZH2 and PRC2 repression signatures correlated positively with inflammatory response genes, including expression of CCL2, CXCL9, and CXCL10 that are important of NK and T cell trafficking into the PDAC TME (FIG. 7a). Moreover, NK and CD8⁺ T cell gene transcript levels were also significantly associated with EZH2 repressed gene expression (FIG. 7a). These results support a relationship between EZH2 activity, inflammatory chemokine signaling, and cytotoxic lymphocyte infiltration in human PDAC.

We then performed immunohistochemical (IHC) staining on formalin-fixed, paraffin-embedded (FFPE) surgically resected tumor specimens from PDAC patients treated at UMass Memorial hospital to assess expression of EZH2 and the NK cell marker NKp46. The 30 patient samples analyzed presented a spectrum of EZH2 expression and NK cell density, with a trend toward low NK cell numbers correlating with high/intermediate EZH2 expression and higher NK cell penetrance correlating with low EZH2 expression (FIG. 7b,c). Patients with low EZH2 expression in their primary PDAC lesions also had significantly increased overall survival compared to patients with high EZH2 PDAC expression (FIG. 7d). Similarly, overall survival was significantly improved in patients with high/intermediate NK cell numbers compared to those with low or absent tumor NK cells (FIG. 7e). Taken together, our work demonstrates that EZH2 is associated with suppression of inflammatory signaling, NK and T cell dysfunction, and reduced survival in murine and human PDAC, and that targeting EZH2 activity can restore long-term innate and adaptive immune-mediated PDAC control.

### Example 9. SMA⁺ Fibroblasts Prevalent in the PDAC TME Promote EZH2-Mediated SASP Blockade and Subsequent NK and T Cell Suppression

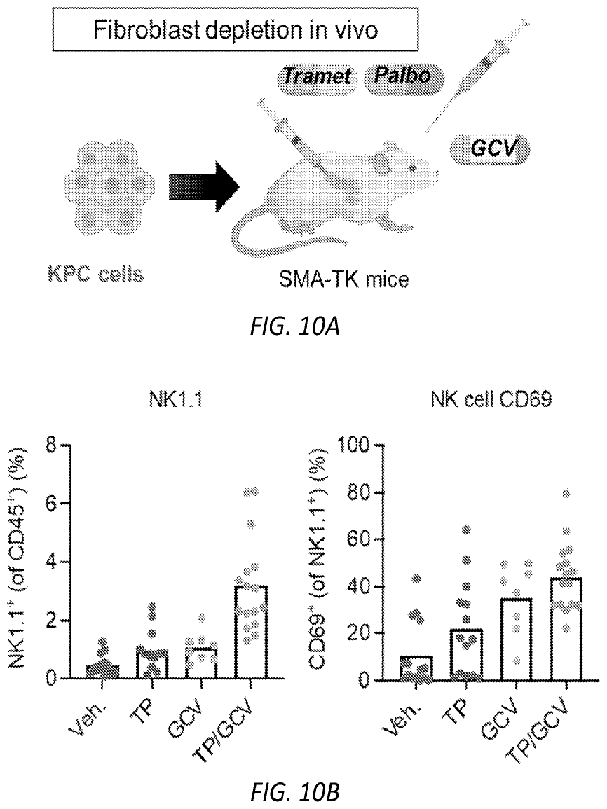

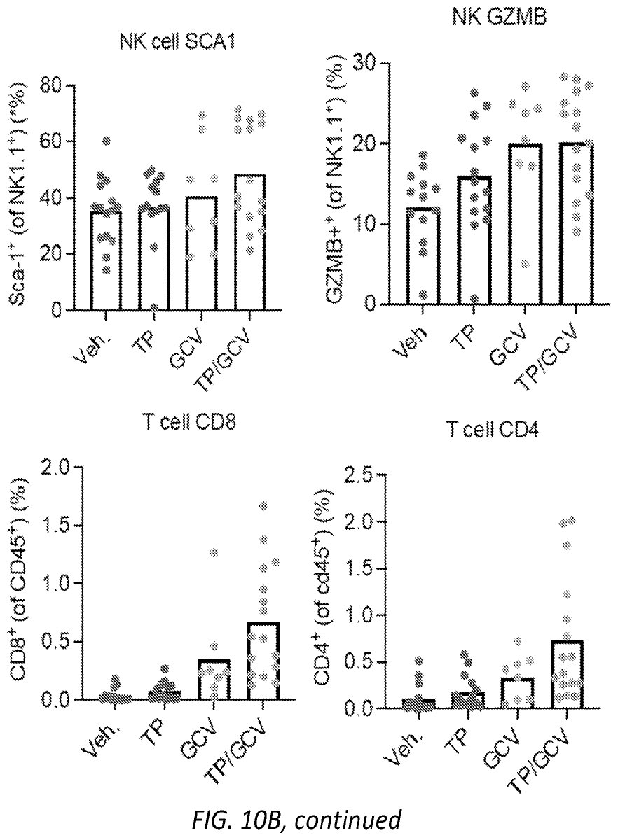

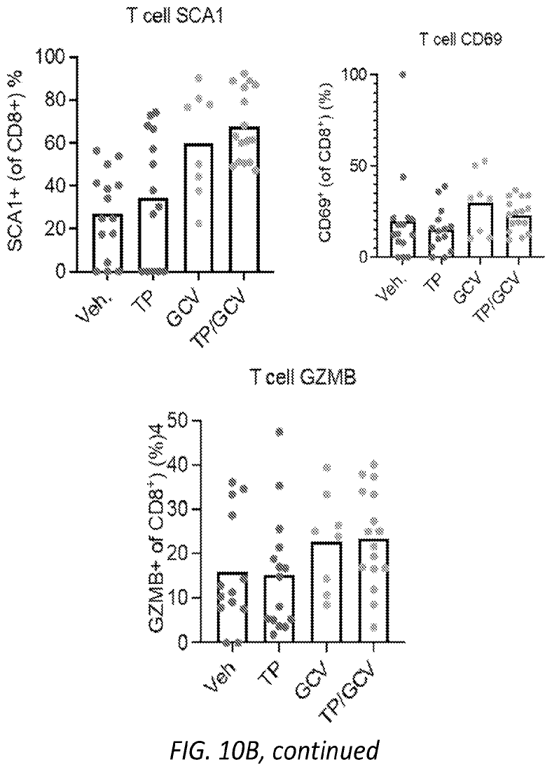

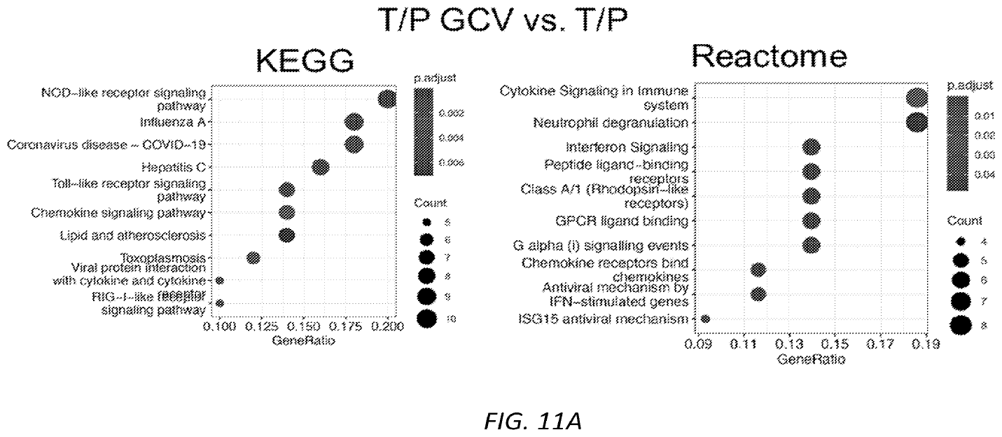

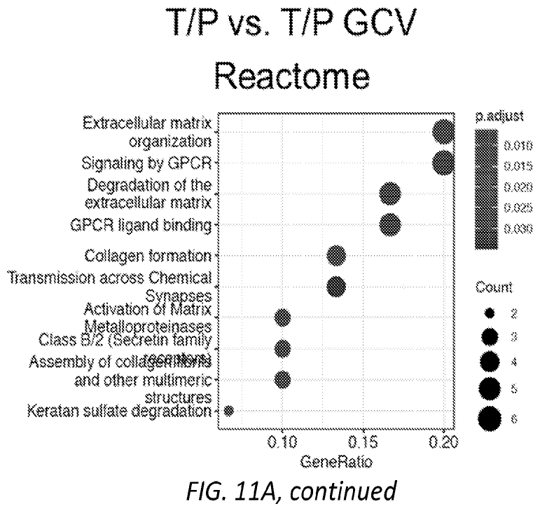

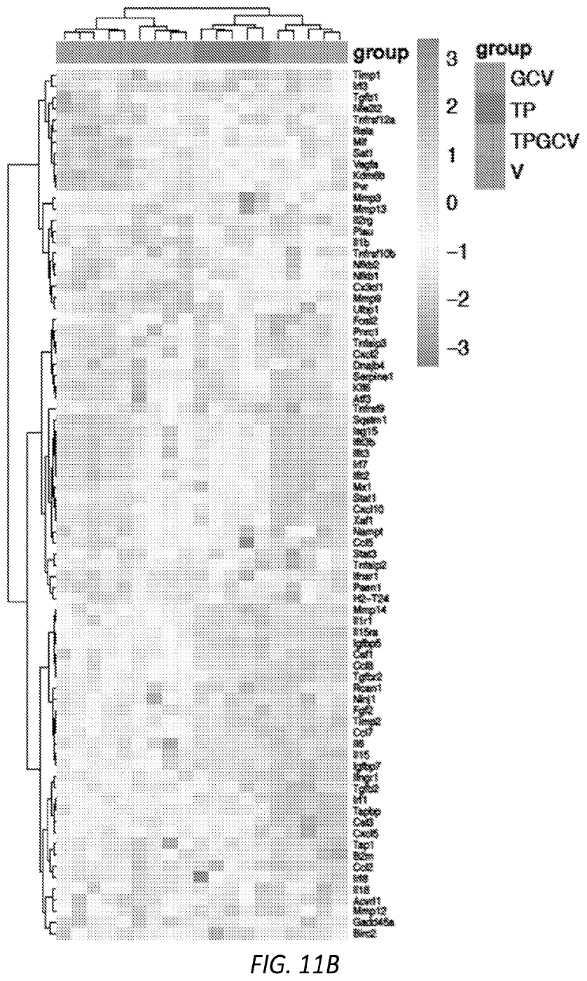

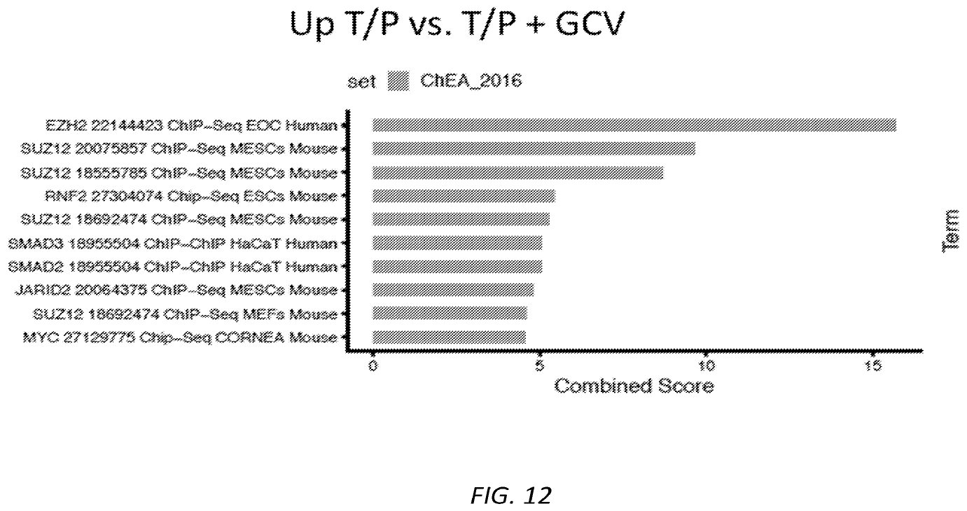

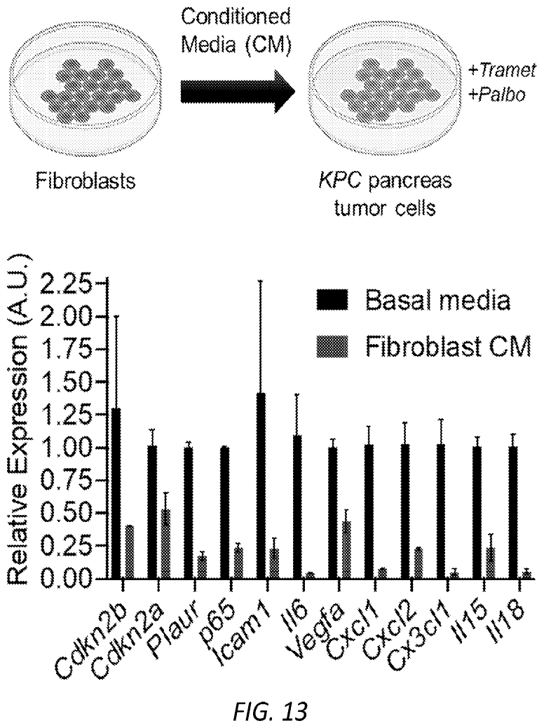

A hallmark of PDAC is a fibrotic and desmoplastic stromal response mediated by secretion of extracellular matrix (ECM) proteins by SMA⁺ myofibroblasts that contributes to poor immune infiltration and responses to immunotherapy. To assess whether SMA⁺ fibroblasts are responsible for SASP inhibition and subsequent NK and T cell suppression specific to the PDAC TME, we took advantage of a previously characterized SMA-TK mouse model (PMID: 24856586) where SMA⁺ fibroblasts can be selectively depleted upon administration of ganciclovir (GCV). Following transplantation of KPC1 cells and subsequent PDAC formation in SMA-TK mice, mice were treated with vehicle, T/P, and/or GCV for 2 weeks to determine the impact on immune responses and SASP output (FIG. 10a). T/P-induced senescence in combination with GCV-mediated SMA⁺ fibroblast depletion led to increased infiltration of NK and CD4⁺ and CD8⁺ T cells and their expression of activation and cytotoxicity markers compared to T/P treatment alone (FIG. 10b). KEGG and Reactome pathway analysis of RNA-seq data from FACS sorted tumor cells revealed a decrease in expression of genes related to ECM organization and collagen formation and an increase in expression of genes related to cytokine, chemokine, and interferon signaling in PDAC tumors treated with T/P and GCV compared with those treated with T/P alone (FIG. 11a). GCV-mediated SMA⁺ fibroblast depletion also led to increased expression of pro-inflammatory SASP genes following T/P-induced senescence (FIG. 11b). Remarkably, GCV-mediated SMA⁺ fibroblast depletion also reduced expression of EZH2 upregulated genes following T/P treatment (FIG. 12); GSEA analysis demonstrated enrichment of EZH2 target genes in T/P vs. T/P+GCV treated tumors. To determine if SASP suppression may be mediated by a fibroblast secreted factor, we subsequently cultured KPC PDAC tumor cells in conditioned media (CM) harvested from an immortalized SMA⁺ pancreatic stellate cell line from a C57BL/6 mouse (FIG. 13). Fibroblast CM significantly blocked the ability of T/P treatment to induce a SASP in senescent PDAC tumor cells in vitro (FIG. 13). Collectively these results show that SMA⁺ fibroblasts (myofibroblasts) contribute to EZH2-mediated SASP and NK and T cell suppression in the pancreatic cancer tumor microenvironment (TME).

### REFERENCES

1 Siegel, R. L., Miller, K. D., Fuchs, H. E. & Jemal, A. Cancer statistics, 2022. CA Cancer J Clin 72, 7-33, doi:10.3322/caac.21708 (2022).
2 Ligorio, M. et al. Stromal Microenvironment Shapes the Intratumoral Architecture of Pancreatic Cancer. Cell 178, 160-175 e127, doi:10.1016/j.cell.2019.05.012 (2019).
3 Olive, K. P. et al. Inhibition of Hedgehog signaling enhances delivery of chemotherapy in a mouse model of pancreatic cancer. Science 324, 1457-1461, doi:10.1126/science.1171362 (2009).
4 Provenzano, P. P. et al. Enzymatic targeting of the stroma ablates physical barriers to treatment of pancreatic ductal adenocarcinoma. Cancer Cell 21, 418-429, doi:10.1016/j.ccr.2012.01.007 (2012).
Brahmer, J. R. et al. Safety and activity of anti-PD-L1 antibody in patients with advanced cancer. N Engl J Med 366, 2455-2465, doi:10.1056/NEJMoa1200694 (2012).
6 Royal, R. E. et al. Phase 2 trial of single agent Ipilimumab (anti-CTLA-4) for locally advanced or metastatic pancreatic adenocarcinoma. J Immunother 33, 828-833, doi:10.1097/CJI.0b013e3181eec14c (2010).
7 Haas, A. R. et al. Phase I Study of Lentiviral-Transduced Chimeric Antigen Receptor-Modified T Cells Recognizing Mesothelin in Advanced Solid Cancers. Mol Ther 27, 1919-1929, doi:10.1016/j.ymthe.2019.07.015 (2019).
8 Bear, A. S., Vonderheide, R. H. & O'Hara, M. H. Challenges and Opportunities for Pancreatic Cancer Immunotherapy. Cancer Cell 38, 788-802, doi:10.1016/j.ccell.2020.08.004 (2020).
9 Waters, A. M. & Der, C. J. KRAS: The Critical Driver and Therapeutic Target for Pancreatic Cancer. Cold Spring Harb Perspect Med 8, doi:10.1101/cshperspect.a031435 (2018).
10 Nguyen, K. B. & Spranger, S. Modulation of the immune microenvironment by tumor-intrinsic oncogenic signaling. J Cell Biol 219, doi:10.1083/jcb.201908224 (2020).
11 Wellenstein, M. D. & de Visser, K. E. Cancer-Cell-Intrinsic Mechanisms Shaping the Tumor Immune Landscape. Immunity 48, 399-416, doi:10.1016/j.immuni.2018.03.004 (2018).
12 Ischenko, I. et al. KRAS drives immune evasion in a genetic model of pancreatic cancer. Nat Commun 12, 1482, doi:10.1038/s41467-021-21736-w (2021).
13 Hamarsheh, S., Gross, O., Brummer, T. & Zeiser, R. Immune modulatory effects of oncogenic KRAS in cancer. Nat Commun 11, 5439, doi:10.1038/s41467-020-19288-6 (2020).
14 Canon, J. et al. The clinical KRAS(G12C) inhibitor AMG 510 drives anti-tumour immunity. Nature 575, 217-223, doi:10.1038/s41586-019-1694-1 (2019).
15 Ribas, A. et al. Combined BRAF and MEK inhibition with PD-1 blockade immunotherapy in BRAF-mutant melanoma. Nat Med 25, 936-940, doi:10.1038/s41591-019-0476-5 (2019).
16 Petroni, G., Buque, A., Zitvogel, L., Kroemer, G. & Galluzzi, L. Immunomodulation by targeted anticancer agents. Cancer Cell 39, 310-345, doi:10.1016/j.ccell.2020.11.009 (2021).
17 Knudsen, E. S. et al. Targeting dual signalling pathways in concert with immune checkpoints for the treatment of pancreatic cancer. Gut 70, 127-138, doi:10.1136/gutjnl-2020-321000 (2021).
18 Ebert, P. J. R. et al. MAP Kinase Inhibition Promotes T Cell and Anti-tumor Activity in Combination with PD-L1 Checkpoint Blockade. Immunity 44, 609-621, doi:10.1016/j.immuni.2016.01.024 (2016).
19 Ruscetti, M. et al. NK cell-mediated cytotoxicity contributes to tumor control by a cytostatic drug combination. Science 362, 1416-1422, doi:10.1126/science.aas9090 (2018).
Ruscetti, M. et al. Senescence-Induced Vascular Remodeling Creates Therapeutic Vulnerabilities in Pancreas Cancer. Cell 181, 424-441 e421, doi:10.1016/j.cell.2020.03.008 (2020).
21 Faget, D. V., Ren, Q. & Stewart, S. A. Unmasking senescence: context-dependent effects of SASP in cancer. Nat Rev Cancer 19, 439-453, doi:10.1038/s41568-019-0156-2 (2019).
22 Rao, S. G. & Jackson, J. G. SASP: Tumor Suppressor or Promoter? Yes!Trends Cancer 2, 676-687, doi:10.1016/j.trecan.2016.10.001 (2016).
23 Coppe, J. P. et al. Senescence-associated secretory phenotypes reveal cell-nonautonomous functions of oncogenic RAS and the p53 tumor suppressor. PLoS Biol 6, 2853-2868, doi:10.1371/journal.pbio.0060301 (2008).
24 Jerby-Arnon, L. et al. A Cancer Cell Program Promotes T Cell Exclusion and Resistance to Checkpoint Blockade. Cell 175, 984-997 e924, doi:10.1016/j.cell.2018.09.006 (2018).
Vilgelm, A. E. et al. Connecting the Dots: Therapy-Induced Senescence and a Tumor-Suppressive Immune Microenvironment. J Natl Cancer Inst 108, djv406, doi:10.1093/jnci/djv406 (2016).
26 Kang, T. W. et al. Senescence surveillance of pre-malignant hepatocytes limits liver cancer development. Nature 479, 547-551, doi:10.1038/nature10599 (2011).
27 Xue, W. et al. Senescence and tumour clearance is triggered by p53 restoration in murine liver carcinomas. Nature 445, 656-660, doi:10.1038/nature05529 (2007).
28 Chibaya, L., Snyder, J. & Ruscetti, M. Senescence and the tumor-immune landscape: Implications for cancer immunotherapy. Semin Cancer Biol, doi:10.1016/j.semcancer.2022.02.005 (2022).
29 Antonangeli, F., Zingoni, A., Soriani, A. & Santoni, A. Senescent cells: Living or dying is a matter of NK cells. Journal of Leukocyte Biology 105, 1275-1283, doi.org/10.1002/JLB.MR0718-299R (2019).
30 Huntington, N. D., Cursons, J. & Rautela, J. The cancer-natural killer cell immunity cycle. Nat Rev Cancer 20, 437-454, doi:10.1038/s41568-020-0272-z (2020).
31 Hingorani, S. R. et al. Preinvasive and invasive ductal pancreatic cancer and its early detection in the mouse. Cancer Cell 4, 437-450 (2003).
32 Jackson, E. L. et al. Analysis of lung tumor initiation and progression using conditional expression of oncogenic K-ras. Genes Dev 15, 3243-3248, doi:10.1101/gad.943001 (2001).
33 Vilgelm, A. E. & Richmond, A. Chemokines Modulate Immune Surveillance in Tumorigenesis, Metastasis, and Response to Immunotherapy. Front Immunol 10, 333, doi:10.3389/fimmu.2019.00333 (2019).
34 Serrano, M., Lin, A. W., McCurrach, M. E., Beach, D. & Lowe, S. W. Oncogenic ras provokes premature cell senescence associated with accumulation of p53 and p16INK4a. Cell 88, 593-602, doi:10.1016/s0092-8674(00)81902-9 (1997).
35 Narita, M. et al. Rb-mediated heterochromatin formation and silencing of E2F target genes during cellular senescence. Cell 113, 703-716, doi:10.1016/s0092-8674(03)00401-x (2003).
36 Chien, Y. et al. Control of the senescence-associated secretory phenotype by NF-kappaB promotes senescence and enhances chemosensitivity. Genes Dev 25, 2125-2136, doi:10.1101/gad.17276711 (2011).
37 Gluck, S. et al. Innate immune sensing of cytosolic chromatin fragments through cGAS promotes senescence. Nat Cell Biol 19, 1061-1070, doi:10.1038/ncb3586 (2017).
38 Kang, C. et al. The DNA damage response induces inflammation and senescence by inhibiting autophagy of GATA4. Science 349, aaa5612, doi:10.1126/science.aaa5612 (2015).
39 Hoare, M. et al. NOTCH1 mediates a switch between two distinct secretomes during senescence. Nat Cell Biol 18, 979-992, doi:10.1038/ncb3397 (2016).
Tasdemir, N. et al. BRD4 Connects Enhancer Remodeling to Senescence Immune Surveillance. Cancer Discov 6, 612-629, doi:10.1158/2159-8290.CD-16-0217 (2016).
41 Laugesen, A., Hojfeldt, J. W. & Helin, K. Role of the Polycomb Repressive Complex 2 (PRC2) in Transcriptional Regulation and Cancer. Cold Spring Harb Perspect Med 6, doi:10.1101/cshperspect.a026575 (2016).
42 Ito, T., Teo, Y. V., Evans, S. A., Neretti, N. & Sedivy, J. M. Regulation of Cellular Senescence by Polycomb Chromatin Modifiers through Distinct DNA Damage—and Histone Methylation-Dependent Pathways. Cell Rep 22, 3480-3492, doi:10.1016/j.celrep.2018.03.002 (2018).
43 Bracken, A. P. et al. The Polycomb group proteins bind throughout the INK4A-ARF locus and are disassociated in senescent cells. Genes Dev 21, 525-530, doi:10.1101/gad.415507 (2007).
44 Yasuda, T. et al. Inflammation-driven senescence-associated secretory phenotype in cancer-associated fibroblasts enhances peritoneal dissemination. Cell Rep 34, 108779, doi:10.1016/j.celrep.2021.108779 (2021).
45 Peng, D. et al. Epigenetic silencing of TH1-type chemokines shapes tumour immunity and immunotherapy. Nature 527, 249-253, doi:10.1038/nature15520 (2015).
46 Zingg, D. et al. The Histone Methyltransferase Ezh2 Controls Mechanisms of Adaptive Resistance to Tumor Immunotherapy. Cell Rep 20, 854-867, doi:10.1016/j.celrep.2017.07.007 (2017).
47 Bugide, S., Gupta, R., Green, M. R. & Wajapeyee, N. EZH2 inhibits NK cell-mediated antitumor immunity by suppressing CXCL10 expression in an HDAC10-dependent manner. Proc Natl Acad Sci USA 118, doi:10.1073/pnas.2102718118 (2021).
48 Bugide, S., Green, M. R. & Wajapeyee, N. Inhibition of Enhancer of zeste homolog 2 (EZH2) induces natural killer cell-mediated eradication of hepatocellular carcinoma cells. Proc Natl Acad Sci USA 115, E3509-E3518, doi:10.1073/pnas.1802691115 (2018).
49 Kaya-Okur, H. S. et al. CUT&Tag for efficient epigenomic profiling of small samples and single cells. Nat Commun 10, 1930, doi:10.1038/s41467-019-09982-5 (2019).
50 Gan, L. et al. Epigenetic regulation of cancer progression by EZH2: from biological insights to therapeutic potential. Biomark Res 6, 10, doi:10.1186/s40364-018-0122-2 (2018).
51 Allavena, P. et al. Induction of natural killer cell migration by monocyte chemotactic protein-1, -2 and -3. European Journal of Immunology 24, 3233-3236, doi.org/10.1002/eji.1830241249 (1994).
52 Iannello, A., Thompson, T. W., Ardolino, M., Lowe, S. W. & Raulet, D. H. p53-dependent chemokine production by senescent tumor cells supports NKG2D-dependent tumor elimination by natural killer cells. J Exp Med 210, 2057-2069, doi:10.1084/jem.20130783 (2013).
53 Litchfield, K. et al. Meta-analysis of tumor- and T cell-intrinsic mechanisms of sensitization to checkpoint inhibition. Cell 184, 596-614 e514, doi:10.1016/j.cell.2021.01.002 (2021).
54 Chow, M. T. et al. Intratumoral Activity of the CXCR3 Chemokine System Is Required for the Efficacy of Anti-PD-1 Therapy. Immunity 50, 1498-1512 e1495, doi:10.1016/j.immuni.2019.04.010 (2019).
55 Harlin, H. et al. Chemokine expression in melanoma metastases associated with CD8+ T-cell recruitment. Cancer Res 69, 3077-3085, doi:10.1158/0008-5472.CAN-08-2281 (2009).
56 Reschke, R. et al. Immune cell and tumor cell-derived CXCL10 is indicative of immunotherapy response in metastatic melanoma. J Immunother Cancer 9, doi:10.1136/jitc-2021-003521 (2021).
57 Hingorani, S. R. et al. Trp53R172H and KrasG12D cooperate to promote chromosomal instability and widely metastatic pancreatic ductal adenocarcinoma in mice. Cancer Cell 7, 469-483, doi:10.1016/j.ccr.2005.04.023 (2005).
58 Moffitt, R. A. et al. Virtual microdissection identifies distinct tumor- and stroma-specific subtypes of pancreatic ductal adenocarcinoma. Nat Genet 47, 1168-1178, doi:10.1038/ng.3398 (2015).
59 Ho, W. J., Jaffee, E. M. & Zheng, L. The tumour microenvironment in pancreatic cancer—clinical challenges and opportunities. Nat Rev Clin Oncol 17, 527-540, doi:10.1038/s41571-020-0363-5 (2020).
60 Ougolkov, A. V., Bilim, V. N. & Billadeau, D. D. Regulation of pancreatic tumor cell proliferation and chemoresistance by the histone methyltransferase enhancer of zeste homologue 2. Clin Cancer Res 14, 6790-6796, doi:10.1158/1078-0432.CCR-08-1013 (2008).
61 Patil, S. et al. EZH2 Regulates Pancreatic Cancer Subtype Identity and Tumor Progression via Transcriptional Repression of GATA6. Cancer Res 80, 4620-4632, doi:10.1158/0008-5472.CAN-20-0672 (2020).
62 Galon, J. & Bruni, D. Approaches to treat immune hot, altered and cold tumours with combination immunotherapies. Nat Rev Drug Discov 18, 197-218, doi:10.1038/s41573-018-0007-y (2019).
63 Spranger, S., Dai, D., Horton, B. & Gajewski, T. F. Tumor-Residing Batf3 Dendritic Cells Are Required for Effector T Cell Trafficking and Adoptive T Cell Therapy. Cancer Cell 31, 711-723 e714, doi:10.1016/j.ccell.2017.04.003 (2017).
64 Wang, J. & Wang, G. G. No Easy Way Out for EZH2: Its Pleiotropic, Noncanonical Effects on Gene Regulation and Cellular Function. Int J Mol Sci 21, doi:10.3390/ijms21249501 (2020).
65 Kim, K. H. et al. SWI/SNF-mutant cancers depend on catalytic and non-catalytic activity of EZH2. Nat Med 21, 1491-1496, doi:10.1038/nm.3968 (2015).
66 Kim, W. et al. Targeted disruption of the EZH2-EED complex inhibits EZH2-dependent cancer. Nat Chem Biol 9, 643-650, doi:10.1038/nchembio.1331 (2013).
67 Tomassi, S., Romanelli, A., Zwergel, C., Valente, S. & Mai, A. Polycomb Repressive Complex 2 Modulation through the Development of EZH2-EED Interaction Inhibitors and EED Binders. Journal of Medicinal Chemistry 64, 11774-11797, doi:10.1021/acs.jmedchem.1c00226 (2021).
68 Hsu, J. H. et al. EED-Targeted PROTACs Degrade EED, EZH2, and SUZ12 in the PRC2 Complex. Cell Chem Biol 27, 41-46 e17, doi:10.1016/j.chembiol.2019.11.004 (2020).
69 Ma, A. et al. Discovery of a first-in-class EZH2 selective degrader. Nat Chem Biol 16, 214-222, doi:10.1038/s41589-019-0421-4 (2020).
70 Wang, J. et al. EZH2 noncanonically binds cMyc and p300 through a cryptic transactivation domain to mediate gene activation and promote oncogenesis. Nature Cell Biology 24, 384-399, doi:10.1038/s41556-022-00850-x (2022).
71 Shao, F. F., Chen, B. J. & Wu, G. Q. The functions of EZH2 in immune cells: Principles for novel immunotherapies. J Leukoc Biol 110, 77-87, doi:10.1002/JLB.1RU0520-311R (2021).
72 Li, C. et al. Finding an easy way to harmonize: a review of advances in clinical research and combination strategies of EZH2 inhibitors. Clin Epigenetics 13, 62, doi:10.1186/s13148-021-01045-1 (2021).
73 Zuber, J. et al. Toolkit for evaluating genes required for proliferation and survival using tetracycline-regulated RNAi. Nat Biotechnol 29, 79-83, doi:10.1038/nbt.1720 (2011).
74 Gopalan, S., Wang, Y., Harper, N. W., Garber, M. & Fazzio, T. G. Simultaneous profiling of multiple chromatin proteins in the same cells. Mol Cell 81, 4736-4746 e4735, doi:10.1016/j.molcel.2021.09.019 (2021).
75 Li, H. & Durbin, R. Fast and accurate short read alignment with Burrows-Wheeler transform. Bioinformatics 25, 1754-1760, doi:10.1093/bioinformatics/btp324 (2009).
76 Li, H. et al. The Sequence Alignment/Map format and SAMtools. Bioinformatics 25, 2078-2079, doi:10.1093/bioinformatics/btp352 (2009).
77 Ross-Innes, C. S. et al. Differential oestrogen receptor binding is associated with clinical outcome in breast cancer. Nature 481, 389-393, doi:10.1038/nature10730 (2012).
78 Love, M. I., Huber, W. & Anders, S. Moderated estimation of fold change and dispersion for RNA-seq data with DESeq2. Genome Biol 15, 550, doi:10.1186/s13059-014-0550-8 (2014).
79 Leek, J. T. svaseq: removing batch effects and other unwanted noise from sequencing data. Nucleic Acids Res 42, doi:10.1093/nar/gku864 (2014).
80 Benjamini, Y. & Hochberg, Y. Controlling the False Discovery Rate: A Practical and Powerful Approach to Multiple Testing. Journal of the Royal Statistical Society. Series B (Methodological) 57, 289-300 (1995).
81 Liberzon, A. et al. Molecular signatures database (MSigDB) 3.0. Bioinformatics 27, 1739-1740, doi:10.1093/bioinformatics/btr260 (2011).
82 Liberzon, A. et al. The Molecular Signatures Database (MSigDB) hallmark gene set collection. Cell Syst 1, 417-425, doi:10.1016/j.cels.2015.12.004 (2015).
83 Yu, G., Wang, L.-G., Han, Y. & He, Q.-Y. clusterProfiler: an R Package for Comparing Biological Themes Among Gene Clusters. OMICS: A Journal of Integrative Biology 16, 284-287, doi:10.1089/omi.2011.0118 (2012).
84 Robinson, J. T. et al. Integrative genomics viewer. Nat Biotechnol 29, 24-26, doi:10.1038/nbt.1754 (2011).
85 Bolger, A. M., Lohse, M. & Usadel, B. Trimmomatic: a flexible trimmer for Illumina sequence data. Bioinformatics 30, 2114-2120, doi:10.1093/bioinformatics/btu170 (2014).
86 Dobin, A. et al. STAR: ultrafast universal RNA-seq aligner. Bioinformatics 29, 15-21, doi:10.1093/bioinformatics/bts635 (2013).
87 Anders, S., Pyl, P. T. & Huber, W. HTSeq—a Python framework to work with high-throughput sequencing data. Bioinformatics 31, 166-169, doi:10.1093/bioinformatics/btu638 (2015).
88 Kanehisa, M., Furumichi, M., Tanabe, M., Sato, Y. & Morishima, K. KEGG: new perspectives on genomes, pathways, diseases and drugs. Nucleic Acids Res 45, D353-D361, doi:10.1093/nar/gkw1092 (2017).
89 Liao, Y., Smyth, G. K. & Shi, W. featureCounts: an efficient general purpose program for assigning sequence reads to genomic features. Bioinformatics 30, 923-930, doi:10.1093/bioinformatics/btt656 (2014).
90 Ihaka, R. & Gentleman, R. R: A Language for Data Analysis and Graphics. Journal of Computational and Graphical Statistics 5, 299-314, doi:10.2307/1390807 (1996).
91 Chen, E. Y. et al. Enrichr: interactive and collaborative HTML5 gene list enrichment analysis tool. BMC Bioinformatics 14, 128, doi:10.1186/1471-2105-14-128 (2013).
92 Yu, J. et al. A polycomb repression signature in metastatic prostate cancer predicts cancer outcome. Cancer Res 67, 10657-10663, doi:10.1158/0008-5472.CAN-07-2498 (2007).
93 Cursons, J. et al. A Gene Signature Predicting Natural Killer Cell Infiltration and Improved Survival in Melanoma Patients. Cancer Immunol Res 7, 1162-1174, doi:10.1158/2326-6066.CIR-18-0500 (2019).
94 Cozar, B. et al. Tumor-Infiltrating Natural Killer Cells. Cancer Discov 11, 34-44, doi:10.1158/2159-8290.CD-20-0655 (2021).
95 Morel, K. L. et al. EZH2 inhibition activates a dsRNA-STING-interferon stress axis that potentiates response to PD-1 checkpoint blockade in prostate cancer. Nat Cancer 2, 444-456, doi:10.1038/s43018-021-00185-w (2021).
96 Chicas et al., Dissecting the unique role of the retinoblastoma tumor suppressor during cellular senescence. Cancer Cell 17, 376-387 (2010).
97 V. Krizhanovsky et al., Senescence of activated stellate cells limits liver fibrosis. Cell 134, 657-667 (2008).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for preventing or treating KRAS mutant pancreatic cancer in a subject in need thereof comprising administering to the subject an effective amount of (i) an effective amount of a PRC2 inhibitor, (ii) an effective amount of a MEK inhibitor, (ii) and an effective amount of a CDK4/6 inhibitor.

2. The method of claim 1, wherein the MEK inhibitor is an inhibitory nucleic acid or a small molecule inhibitor, preferably selected from the group consisting of trametinib, cobimetinib, binimetinib, selumetinib, PD-325901, TAK-733, Cl-1040 (PD 184352), PD0325901, MEK162, AZD8330, GDC-0623, refametinib, pimasertib, R04987655, R05126766, WX-554, HL-085, CInQ-03, G-573, PD184161, PD318088, PD98059, R05068760, U0126, and SL327.

3. The method of claim 1, wherein the CDK4/6 inhibitor is an inhibitory nucleic acid or a small molecule inhibitor, preferably selected from the group consisting of palbociclib, ribociclib, and abemaciclib.

4. The method of claim 1, wherein the PRC2 inhibitor is an inhibitory nucleic acid or a small molecule inhibitor, optionally an inhibitor of EZH2 or EED as described herein, preferably GSK126 or Tazemetostat.

5. The method of claim 1, wherein the subject is non-responsive to at least one prior line of cancer therapy.

6. The method of claim 4, wherein the at least one prior line of cancer therapy is chemotherapy or immunotherapy.

7. The method of claim 1, wherein the pancreatic cancer is an exocrine pancreatic cancer or an endocrine pancreatic cancer.

8. The method of claim 1, wherein the pancreatic cancer is selected from the group consisting of pancreatic ductal adenocarcinoma (PDAC), acinar cell carcinoma, solid pseudopapillary neoplasms, pancreatoblastoma, pancreatic neuroendocrine tumors (PNETs), gastrinomas, insulinomas, glucagonomas, somatostatinomas and VIPomas.

9. The method of claim 1, wherein the KRAS mutation is G12D, G12V, G12C, G12R, G12A, G13D, Q61L or Q61H.

10. The method of claim 1, wherein the PRC2 inhibitor, MEK inhibitor, and CDK4/6 inhibitor are administered sequentially, simultaneously, or separately.

11. The method of claim 1, wherein the PRC2 inhibitor, MEK inhibitor, and/or CDK4/6 inhibitor is administered orally, intraperitoneally, or intravenously.

12. The method of claim 1, wherein the subject is human.

13. The method of claim 1, wherein the subject exhibits an increase in one or more of (a) NK cell immune surveillance, (b) senescent tumor cell clearance, or (c) vascular re-normalization after administration of the PRC2 inhibitor, MEK inhibitor, and CDK4/6 inhibitor.

14. The method of claim 1, wherein the subject exhibits a delay in metastatic onset and/or tumor growth after administration of the PRC2 inhibitor, MEK inhibitor, and CDK4/6 inhibitor compared to that observed in an untreated control subject diagnosed with pancreatic cancer.

15. The method of claim 1, further comprising administering at least one chemotherapeutic agent in a patient with pancreatic cancer comprising.

16. The method of claim 15, wherein the at least one chemotherapeutic agent is selected from the group consisting of abraxane, capecitabine, erlotinib, fluorouracil (5-FU), gemcitabine, irinotecan, leucovorin, nab-paclitaxel, cisplatin, irinotecan, docetaxel, oxaliplatin, tipifamib, everolimus, sunitinib, dovitinib, ruxolitinib, pegylated-hyaluronidase, pemetrexed, folinic acid, paclitaxel, MK2206, GDC-0449, IPI-926, gamma secretase/RO4929097, M402, and LY293111.

17. The method of claim 1, further comprising administering at least one immunotherapeutic agent.

18. The method of claim 17, wherein the at least one immunotherapeutic agent is selected from the group consisting of immune checkpoint inhibitors, sipuleucel-T, CRS-207, and GVAX, or is a monoclonal antibody selected from ipilimumab, 90Y-Clivatuzumab tetraxetan, pembrolizumab, nivolumab, trastuzumab, cixutumumab, ganitumab, demcizumab, cetuximab, nimotuzumab, and dalotuzumab.

19. A kit comprising a PRC2 inhibitor, a MEK inhibitor, a CDK4/CDK6 inhibitor, and instructions for treating pancreatic cancer.

20. A pharmaceutical composition comprising a PRC2 inhibitor and one or both, preferably both, of a MEK inhibitor and/or a CDK4/6 inhibitor, and a pharmaceutically acceptable excipient or carrier.
